(19) United States
(12) Patent Application Publication
(10) Pub. No.: US 2025/0333714 A1
(43) Pub. Date: Oct. 30, 2025

(54) ARTIFICIAL DNA REPLISOME AND METHODS OF USE THEREOF

(71) Applicant: Regents of the University of Minnesota, Minneapolis, MN (US)

(72) Inventors: Romas KAZLAUSKAS, Minneapolis, MN (US); Michael TRAVISANO, Minneapolis, MN (US); Xiao YI, Minneapolis, MN (US)

(73) Assignee: Regents of the University of Minnesota, Minneapolis, MN (US)

(21) Appl. No.: 18/277,444

(22) PCT Filed: Feb. 17, 2022
(86) PCT No.: PCT/US2022/016854

§ 371 Date: Aug. 16, 2023

Related U.S. Application Data
(60) Provisional application No. 63/150,374, filed on Feb. 17, 2021.

(51) Int. Cl. C12N 9/12; C12N 9/22; C12N 9/90; C12N 15/10

(52) U.S. Cl. CPC C12N 9/1252; C12N 9/22; C12N 9/90; C12N 15/1024; C07K 2319/00; C12Y 207/07007; C12Y 306/04012

(57) ABSTRACT

Certain embodiments of the invention provide a recombinant polypeptide comprising a T5 DNA polymerase amino acid sequence operably linked to a DNA helicase amino acid sequence, as well as methods of using such a recombinant polypeptide for DNA replication and/or mutagenesis. Certain embodiments of the invention provide a targeted artificial DNA replisome complex. Certain embodiments of the invention provide a targeted DNA mutagenesis system.

### CROSS REFERENCE TO RELATED APPLICATION

This application claims priority to U.S. Provisional Application No. 63/150,374 filed on 17 Feb. 2021. The entire content of the application referenced above is hereby incorporated by reference herein.

### SEQUENCE LISTING

The instant application contains a Sequence Listing which has been submitted electronically in ASCII format and is hereby incorporated by reference in its entirety. Said ASCII copy, created on Mar. 25, 2022, is named 09531_528W01_SL.txt and is 160,797 bytes in size.

### BACKGROUND OF THE INVENTION

New molecular functions evolve in nature, but this process requires decades and is difficult to direct to non-natural goals. One alternative is directed evolution where in vitro manipulations create mixtures or a library of DNA that is transferred into microbes. Proteins are then expressed in microbes for screening or selection to identify the improved variants. The inefficiency of the transfer of DNA into microbes limits this approach to about a million variants, which is only a small fraction of the possibilities. Thus, there is a need for new compositions and methods for directed evolution that have improved efficiency for evolving desired molecular functions.

### SUMMARY OF THE INVENTION

Certain embodiments of the invention provide a recombinant polypeptide comprising a T5 DNA polymerase amino acid sequence operably linked to a DNA helicase amino acid sequence (e.g., Rep helicase, or a fragment thereof).

Certain embodiments of the invention provide a T5 DNA polymerase comprising a I308V mutation, wherein the substitution and position are in reference to SEQ ID NO:1.

Certain embodiments of the invention provide a recombinant polypeptide comprising a T5 DNA polymerase that comprises a I308V mutation, wherein the substitution and position are in reference to SEQ ID NO:1.

Certain embodiments of the invention provide a nucleic acid encoding a recombinant polypeptide as described herein.

Certain embodiments of the invention provide an expression cassette comprising a nucleic acid as described herein.

Certain embodiments of the invention provide a helper vector comprising a nucleic acid sequence as described herein or an expression cassette as described herein.

Certain embodiments of the invention provide a method comprising contacting a cell with a nucleic acid as described herein, an expression cassette as described herein, or a helper vector as described herein.

Certain embodiments of the invention provide a DNA replisome complex, comprising:
1) a target double stranded DNA (dsDNA) comprising a DNA nickase initiation sequence;
2) a corresponding DNA nickase that is capable of nicking the target dsDNA at the initiation sequence; and
3) a recombinant polypeptide as described herein,
wherein the recombinant polypeptide is capable of replicating the nicked dsDNA.

Certain embodiments of the invention provide a nucleic acid sequence comprising a target double stranded DNA (dsDNA) as described herein.

Certain embodiments of the invention provide a target vector comprising a nucleic acid sequence as described herein, or an expression cassette as described herein.

Certain embodiments of the invention provide a host cell comprising a DNA replisome complex as described herein; a DNA nickase or a vector encoding a DNA nickase; a helper vector as described herein; and/or a target vector as described herein.

Certain embodiments of the invention provide a targeted DNA replication or mutagenesis system comprising:
1) a target dsDNA comprising a DNA nickase initiation sequence, or a target vector as described herein;
2) a DNA nickase, or a host cell comprising the DNA nickase; and
3) a recombinant polypeptide as described herein or a helper vector as described herein;
   wherein the DNA nickase is capable of nicking the dsDNA or the target vector at the initiation sequence and the recombinant polypeptide is capable of replicating or mutagenizing the nicked dsDNA or target vector.

Certain embodiments of the invention provide a kit comprising:
1) a target vector comprising a DNA nickase initiation sequence;
2) a host cell comprising a DNA nickase (e.g., CisA);
3) a helper vector as described herein;
4) packaging materials; and
   instructions for inserting a target DNA sequence into the target vector downstream of the DNA nickase initiation sequence; and contacting the host cell with the helper vector and the target vector comprising the target DNA sequence, under conditions suitable for the vectors to enter the host cell and mutagenize the target vector.

Certain embodiments of the present invention also provide a targeted mutagenesis method that comprises contacting a dsDNA with a DNA nickase, and contacting the nicked dsDNA with a DNA helicase and an error-prone DNA polymerase. In certain embodiments, the DNA helicase and the error-prone DNA polymerase are operably linked to form a recombinant polypeptide.

Certain embodiments of the invention provide a method of mutagenizing a target DNA sequence comprising introducing the target DNA sequence into a target dsDNA downstream of a DNA nickase initiation sequence, and assembling a DNA replisome complex as described herein.

Certain embodiments of the invention provide a method of mutagenizing a target DNA sequence in a cell comprising contacting the target DNA sequence with a recombinant polypeptide as described herein, wherein the target DNA sequence is operably linked downstream of a DNA nickase initiation sequence; and wherein the cell expresses a corresponding DNA nickase; under conditions suitable for the DNA nickase to nick the initiation sequence and for the recombinant polypeptide to mutagenize the target DNA sequence.

Certain embodiments of the invention provide a method of mutagenizing a target DNA sequence comprising contacting a host cell that expresses a DNA nickase with: 1) a target vector comprising a corresponding DNA nickase initiation sequence operably linked to the target DNA sequence; and 2) a helper vector as described herein; under conditions suitable for the vectors to enter the host cell; for the DNA nickase to nick the initiation sequence; and for the recombinant polypeptide to mutagenize the target DNA sequence.

Certain embodiments provide a nucleic acid as described herein.

Certain embodiments provide an expression cassette as described herein.

Certain embodiments provide a vector described herein.

Certain embodiments provide a cell, such as a host cell, as described herein (e.g., comprising one or more nucleic acids, expression cassettes or vectors described herein).

### BRIEF DESCRIPTION OF THE FIGURES

FIGS. 1A-1D. A targeted error-prone artificial DNA replisome. FIG. 1A. The two components of TADR, cisA targeted nickase from phage PhiX174 and the fusion of T5 DNA polymerase from phage T5 and Rep helicase from bacterium E. coli, are expressed from chromosome and helper plasmid, respectively. A mutant Rep helicase is used with deletion of 33 C-terminal amino acid residues. The two parallel bars denote double-stranded DNA; oval, CisA protein; wedge, Rep helicase domain of the fusion protein; circle, DNA polymerase domain; line, linker. Promoters are indicated with arrows and the names. The chromosomal rep of the host cell was knocked out. FIG. 1B. Proposed steps of in-vivo targeted mutagenesis by an artificial replisome. The three neighboring blocks in the center (from left to right) indicate initiation, gene(s) of interest, and termination sequences, respectively. Dashes mark nascent DNA; the bulge marks the mutation introduced from synthesis. FIG. 1C. Proposed arrangement of proteins in the artificial replisome. The proposed location of the CisA protein (upper right oval) relative to the ribbon diagram of the X-ray structure of Rep helicase (PDB: 1AUU) (Korolev et al., Cell, 90 (4): 635-647, (1997)) is based on a structure of a homologous protein complex (Carr et al., Nucleic Acids Research, 44 (5): 2417-2428, (2016)). The DNA polymerase (lower left oval) is fused via a linker (light gray curve) to the N-terminus of Rep helicase. Arrow indicates direction of the replisome translocating on DNA. FIG. 1D. External force has little effect on the speed of DNA unwinding by Rep helicase (white dots/line) but slows T7 phage DNA polymerase (dark dots/line). Experimental data were retrieved from (Maier et al., PNAS, 97 (22): 12002-12007, (2000); Arslan et al., Science (New York, N.Y.), 348 (6232): 344-347, (2015)), and fitted by a linear and an exponential model, respectively. Dashed line indicates the intersection of the two speed profiles, marking the force required for both enzymes to act at the same speed.

FIGS. 2A-2C. Testing in vivo functionality of TADR components. FIG. 2A. CisA nicking of the target plasmid. Picture of gel electrophoresis is shown in the bottom with different species of plasmid marked. Quantification of the nicked fraction by image analysis is shown on the top. Star indicates statistical significance with a of 0.05, Student's t-Test and n=3 independently prepared and measured biological replicates. N.S., not significant. Error bars, standard deviation. FIG. 2B. Rep fused to DNA polymerase to alleviate sensitivity of rep-cells to nalidixic acid. Picture of colony growth of serially-diluted cultures is shown (dilution factor of 10, starting from no dilution). Colony growths of 10-fold dilution (second row from the top) were quantified by brightness. An expanded picture with three replicates is shown in FIG. 8. Medium: LB supplemented with 2.5 pg/ml nalidixic acid. Statistics is per 2A. Error bar, standard deviation. FIG. 2C. Mutagenesis on the target. After overnight liquid growth of cells to full density, during which mutations also accumulated, cultures from each sample were plated on LB supplemented with 30 μg/ml kanamycin. Colonies were counted after 16 h incubation. The frequency of kanamycin-resistant colonies per cell plated is reported. Statistics is per 2A except n=5 independently prepared and measured biological replicates. Error bar, standard deviation.

FIGS. 3A-3E. Development and characterization of TADR. FIG. 3A. Mutagenic capacity of TADR with different versions of the helper plasmid. The conditions were per FIG. 2C. RepAC33 was used. Star indicates statistical significance with a of 0.05, Student's t-Test and n=5 or 6 independently prepared and measured biological replicates. Error bar, standard deviation. FIG. 3B. Structure of T5 DNA polymerase was modeled using Robetta server (Kim et al., Nucleic Acids Research, 32 (Web Server issue): W526-W531, (2004)). The evolved I308V was within the exonuclease domain; ancestor mutations D164 and E166 of T5 DNA polymerase are involved in exonuclease activity and A593 is involved in substrate recognition, respectively. FIG. 3C. Performance of TADR was measured by its fold change in on- and off-target mutations over non-TADR. Non-TADR: wildtype E. coli with the target plasmid carrying kanR*. TADR: TADR cells with the same target plasmid and the helper plasmid carrying RepAC33, 1308V and modified 5′UTR. The on-target fold change was measured using kanamycin-resistant colonies; off-target fold change, using streptomycin-resistant and rifampicin-resistant colonies separately. Statistics is per 3A except n=6 independently prepared and measured biological replicates. Error bar, standard deviation. N.S., not significant. Dashed line indicates the level of non-TADR. FIG. 3D. Mutation density across the whole target vector plasmid. Black ball, initiation sequence; white ball, termination sequence; the single fragment (the single fragment on the top) between the initiation sequence (black ball) and the termination sequence (white ball) indicates the target region; the arrow, promoter; the lower left fragment, origin of plasmid replication. Black solid line curve depicts the average mutation density of three biological replicas; two dashed curves, standard deviation. FIG. 3E. Mutational spectrum of TADR (n=3 independently prepared and measured biological replicates. error bar, standard deviation) and other error-prone DNA polymerases.

FIGS. 4A-4B. Evolutionary innovation by TADR: optimizing an efflux pump with expanded substrate repertoire and reduced cellular toxicity. FIG. 4A. Trajectories of phenotypic adaptation. TADR with RepAC33, 5′ UTR modified and I308V was used. Starting from the ancestor, three parallel populations were subjected to the first round of mutagenesis-selection on LB supplemented with 4 ng/pl tigecycline. One big colony was selected from each population and numbered 1, 2, 3. #1 gave bigger colonies than the other two and thus was used as the ancestor for a second round of mutagenesis-selection on LB supplemented with 8 ng/pl tigecycline. One big colony was selected from each of two populations and numbered 1-1, 1-2. The efflux pump gene from five isolates was each PCR-amplified and re-introduced to the unevolved backbone of target plasmid. This treatment purged the potential complicating effect of adaptive mutations that occurred off-target during evolution. The reconstructed evolved target plasmids along with the ancestor were transformed into cells, and growth rates in no antibiotic, in 8 ng/pl tetracycline and 8 ng/pl tigecycline were measured. Each dot was an average of five independently prepared and measured biological replicates; error bar, standard error. FIG. 4B. Structure of the efflux pump was modeled using Phyre2 server (Kelley et al., Nature Protocols, 10 (6): 845-858, (2015)) with the intensive mode. Residues mutated during evolution were marked black.

FIG. 5. The full system of Exemplary TADR. This embodiment consists of three components: the CisA gene expressed from chromosome, the helper vector plasmid that expresses the fused protein and the target vector plasmid that carries the DNA to be mutated. Graph shape of proteins is as per FIG. 1A.

FIG. 6. Steps of self-evolution. Graph shape of proteins is as per FIG. 1A. In this example, target block includes the kanR* gene, which had one nucleotide substitution that inactivated kanamycin resistance. Lightnings indicate mutations created by TADR. The steps to create mutations (1 and 3) were realized by simply growing cells in liquid culture. As cells grow, mutations accumulated on the target regions. This feature allowed tuning stringency of selection for mutation rate. Specifically, growth in step 3, between electroporation and selection by kanamycin, was shortened to five minutes from a full day as in the standard protocol of inducing mutations, an overnight for colony to develop plus twelve to sixteen hours for culture to grow to full density.

FIG. 7. Chemical structures of tetracycline (upper) and tigecycline (bottom).

FIG. 8. Testing in vivo function of Rep fused to T5 DNA polymerase. The condition is the same as in FIG. 2B. Colonies from the second row from the top were analyzed to generate the plot in FIG. 2B.

FIG. 9. Growth rates of cells with ancestral and evolved tetA efflux pump. Data is the same in FIG. 4A but presented with bar-graph. Star indicates statistical significance between the ancestor and the corresponding mutant, with ANOVA, α=0.05 and n=5. Error bar, standard error.

FIG. 10. Secondary structures of transcripts for synonymous mutations in Mutants 1-1 and 1-2 in comparison to those of the ancestor (WT). Arrows mark the mutated nucleotides in both the mutants and wildtype. Predicted free energies (dG) of the secondary structures are also provided. Compared to the wildtype, both mutations led to increase in dG, thereby destabilizing the secondary structures. The secondary structures and their corresponding dG's are computed by Mfold server with default parameter values (Ravikumar, et al., Nat. Chem. Biol. 10, 175-177, (2014)). Figure discloses SEQ ID NOS: 45-48, respectively, in order of appearance.

FIG. 11. The relative speed of DNA polymerase to helicase is critical to a processive and functional replication fork. The plots are per FIG. 1C. When the speed of DNA polymerase is faster, the two curves intersect (vertical dashed line), at which point the replisome is stabilized and becomes processive. When the helicase is faster, the two curves do not intersect, the two motor proteins are discoordinated, and the replisome is unstable and unfunctional.

FIG. 12. Sanger sequencing of evolved target vector plasmids from double reversion assay. Blank bars mark the start and stop codons of the chloramphenicol resistance gene; black bars, the two designed stop codons to inactivate resistance to chloramphenicol. Only if both these designed stop codons were simultaneously mutated to amino acid-encoding codons can chloramphenicol-resistant colonies emerge. Sequences of three evolved target plasmids selected on LB plate supplemented with chloramphenicol are aligned to that of the un-evolved target plasmid. Boxes indicate mutations. Letters above the black bars indicate the wildtype nucleotides mutated to form the designed stop codons. Although precise reversion to the wildtype nucleotides were not observed, in all three evolved target plasmids, both designed stop codons were mutated to amino acid-encoding codons. This result confirmed the capacity of TADR to explore the space of protein sequence with large step-size: Introducing two beneficial mutations at a time. Figure discloses SEQ ID NOS: 49-52, respectively, in order of appearance.

FIG. 13. Multiple sequence alignments highlight the residues to be mutated for increase in error rate. D164 and E166, and A593 of T5 DNA polymerase are involved in exonuclease activity and substrate recognition, respectively. Substitution of these residues increases the error rate in other polymerases (Morrison et al., PNAS, 88 (21): 9473-9477, (1991); Camps et al., PNAS, 100 (17): 9727-9732, (2003)). Letters (164A, 166A and 593R) above the sequence indicate substitutions designed in the error-prone T5 DNA polymerase in TADR. Figure discloses SEQ ID NOS: 53-58 and 38, respectively, in order of appearance.

FIG. 14. Mutational density across the mutated target plasmid. Data is the same as in FIG. 3D. Normalized mutational density is defined as the number of mutations normalized to the total mutations of the sample per 200-bp window across the entire plasmid. Density profiles for three biological replicates are shown individually. 264 mutations were called in the total of these three samples. See Methods in Example section for details. Line fragments in the bottom indicate different parts (Origin of replication in the left block, Promoter in black block, Target region in the right block) of target plasmid. The downward vertical arrow indicates the site of transcriptional termination. Vertical dashed lines map the peaks to their respective plasmid parts.

FIG. 15. Comparing on- and off-target mutagenetic capacity of TADR under different conditions. The most potent version of TADR was used, with RepAC33, 1308V and modified 5′ UTR. The left panel is the same as FIG. 3c. Mutagenesis was measured with cells grown in minimal medium (left panel) and rich medium of LB (right panel). In LB, the on-target mutation rate of TADR was 4.3×10⁵-fold higher than the non-TADR baseline, higher than the 2.3×10⁵ fold increase in minimal media but not statistically significant (ANOVA, n=6, P=0.15). To measure off-target mutagenesis in LB, two methods were used. The first was as per FIG. 2c, in which the target plasmid without initiation sequence was used. The off-target mutation rate increased 9854-fold, which corresponded to selectivity of 43-fold for mutagenesis on the target plasmid. The second method simply selected cells from the treatment with the full TADR on rifampicin as per FIG. 3c and the left panel. The off-target mutation rate increased 1315-fold, which corresponded to selectivity of 326-fold for mutagenesis on the target plasmid. The discrepancy of 7.5-fold in off-target mutagenesis measured by the two methods was statistically significant (ANOVA, n=6, P=0.012) and was explained by the titration scenario discussed in the Example 1. Cells of the off-target control without initiation sequence could not titrate the fusion protein by binding to its target plasmid; and the excessive fusion protein free in the cytoplasm caused more mutations in the genome.

FIG. 16. On-target mutagenesis can be turned down 112-fold. The most potent version of TADR was used, with RepAC33, I308V and modified 5′ UTR, and the target plasmid with kanR* was used. Cells were grown in minimal media as per FIG. 3c. To turn on TADR, glycerol was used as the only carbon source. To turn down mutagenesis, glycerol was substituted with glucose to suppress expression of the fusion protein. The fusion protein was transcribed by T7 RNA polymerase whose genes was under the glucose-suppressible pBAD promoter. Star indicates statistical significance (ANOVA, n=6, P=0.028) FIG. 17. Replotting FIG. 14 with high binning resolution (bin size, 25 bp). The horizontal bar near the center indicates origin of replication. Numbers along the axis indicate plasmid coordinates in bp. The mutational hotspots are concentrated narrowly in a few bins. Interestingly, the narrow hotspot at the origin of replication happens to peak around the primer promoter for initiation of p15A plasmid replication (the target plasmid is a p15A plasmid). Transcription from this promoter is the very first step of initiation of plasmid replication, which produces a RNA primer to prime DNA polymerase for copying plasmid DNA (Selzer, et al., PNAS. 79, 7082-7086 (1982)). The mutational hotspots are also places transcription initiates or terminates. Transcription initiation and termination are both known to require RNA polymerase to pause/dwell for substantial amount of time, providing a good occasion to stall TADR replisome and induce errors.

FIG. 18. Growths of cells with full TADR system and the same cells but whose target plasmid is without termination sequence. For each treatment, helper plasmid and target plasmid were electroporated into TADR cells, and selected on LB agar plate for both plasmids at 37° C. The full TADR group gave rise to normal colonies after overnight incubation. But the termination sequence-minus group gave rise to small colonies. After extended incubation for these colonies to grow large enough, Cells from both groups were inoculated directly from colonies to liquid LB and incubated at 37° C., shaken at 225 rounds per min. Roughly the same amount of cells from each group were used for the inoculation. Optical density was measured 12 hours after inoculation.

FIG. 19. More details of TADR. Expression of TADR is regulated by catabolite repression. Promoters are indicated with their names. The promoters pLac and pBAD are both catabolite-repression promoters, enabling switch-on/off in FIG. 16: using glucose as the sole carbon source suppresses TADR; and using glycerol as the sole carbon source allows expression of TADR.

### DETAILED DESCRIPTION

Compared to in vitro approaches, in vivo directed evolution approaches, where the mutations are generated to the DNA within the cell, can make a million-fold more variants. Thus, extensive exploration of a protein's sequence space for improved or new molecular functions requires in vivo evolution with large populations. Nonetheless, disentangling the evolution of a target protein from the rest of the proteome is challenging. Described herein, the present invention relates to an engineered multi-component DNA replication complex that acts as a Targeted Artificial Replisome (TADR) in live cells to processively replicate an arbitrary target gene with errors. In certain embodiments, a TADR as described herein enhanced mutagenesis of target genes up to 2.3×10⁵-fold with only a 78-fold increase in off-target mutations.

### DNA Polymerase-Helicase Recombinant Polypeptide

Certain embodiments of the present invention provide a recombinant polypeptide comprising a DNA polymerase amino acid sequence operably linked to a DNA helicase amino acid sequence. The DNA polymerase amino acid sequence and the DNA helicase amino acid sequence are functionally active sequences (e.g., catalytically active). Thus, the recombinant polypeptide may be used to unwind and replicate DNA in a concerted manner.

In certain embodiments of the present invention, the DNA helicase is slower than the DNA polymerase, but the DNA helicase may proceed along the DNA template at about a constant rate to unwind double helix DNA and can tolerate a DNA polymerase collision. Thus, certain embodiments of the invention provide a recombinant polypeptide comprising a DNA polymerase operably linked to a DNA helicase, wherein the DNA polymerase has a higher speed compared to the DNA helicase.

In certain embodiments of the present invention, the coordinated action between the fused DNA helicase and the DNA polymerase improves the efficiency of DNA replication and/or prevents the DNA replisome complex from disassembling prematurely.

In certain embodiments, a recombinant polypeptide described herein is capable of replicating DNA in vivo. In certain embodiments, the recombinant polypeptide described herein is capable of replicating DNA in vitro.

In certain embodiments, a recombinant polypeptide described herein further comprises an optional tag sequence (e.g., 6×His tag (SEQ ID NO: 37)). In certain embodiments, the tag sequence can facilitate purification or detection and is located, e.g., at the N-terminus and/or C-terminus of the recombinant polypeptide. In certain embodiments, the recombinant polypeptide is free of a tag sequence (e.g., 6×His tag (SEQ ID NO: 37)) at the N-terminus and/or C-terminus.

### DNA Polymerase

In certain embodiments, the recombinant polypeptide described herein comprises a high-speed DNA polymerase that is faster than the operably linked DNA helicase. For example, when the DNA helicase has a speed of about 144 base-pairs/s, it may be preferable that the DNA polymerase has a speed higher than 144 base-pairs/s. Without wishing to be bound by theory, a faster DNA polymerase would be able to keep up with the DNA helicase, minimizing the space between the two molecules, and reducing the exposure of newly unwound single-strand DNA to any undesirable exonuclease mediated digestion. In certain embodiments, the high-speed DNA polymerase has an average speed of at least 50 base-pairs/s. In certain embodiments, the high-speed DNA polymerase has an average speed of at least 100 base-pairs/s. In certain embodiments, the high-speed DNA polymerase has an average speed of at least 144 base-pairs/s. In certain embodiments, the high-speed DNA polymerase has an average speed of at least 150 base-pairs/s. In certain embodiments, the high-speed DNA polymerase has an average speed of at least 200 base-pairs/s. In certain embodiments, the high-speed DNA polymerase has an average speed of about 140, 150, 160, 170, 180, 190, 200, 210, 220, 230, 240, or more base-pairs/s. In certain embodiments, the high-speed DNA polymerase has an average speed of about 200 base-pairs/s. In certain embodiments, the high-speed DNA polymerase described herein is an error-prone DNA polymerase.

In certain embodiments, the DNA polymerase is a high processivity polymerase. Processivity is defined as the number of nucleotides being processed in a single DNA binding event (continuous DNA synthesis on a template DNA without dissociation). For example, a high processivity polymerase can process on average at least 100 bp without dissociation from template DNA. Thus, high processivity DNA polymerases are suitable for efficient amplification of long templates. In certain embodiments, the target DNA sequence is about 100 bp to 10 kb, 200 bp to 9.5 kb, 300 bp to 9 kb, 400 bp to 8.5 kb, 500 bp to 8 kb, 600 bp to 7.5 kb, 700 bp to 7 kb, 800 bp to 6.5 kb, 900 bp to 6 kb, 1 kb to 5.5 kb, 1.5 to 5 kb, 2 kb to 4.5 kb, 2.5 kb to 4 kb, or 3 kb to 3.5 kb in length. In certain embodiments, the target DNA sequence is about 300 bp to 6 kb. In certain embodiments, the target DNA sequence is about 500 bp to 5 kb. In certain embodiments, the target DNA sequence is about 700 bp to 4 kb. In certain embodiments, the target DNA sequence is about 800 bp to 3 kb. In certain embodiments, the target DNA sequence is at least 100 bp, 200 bp, 300 bp, 400 bp, 500 bp, 600 bp, 700 bp, 800 bp, 900 bp, 1 kb, 1.5 kb, 2 kb, 2.5 kb, 3 kb, 3.5 kb, 4 kb, 4.5 kb, 5 kb, 6.5 kb, 7 kb, 7.5 kb, 8 kb, 8.5 kb, 9 kb, 9.5 kb, 10 kb or more in length. In certain embodiments, the target DNA sequence is at least 500 bp, 600 bp, 700 bp, 800 bp, 900 bp, or 1 kb in length. In certain embodiments, the target DNA sequence is at least 700 bp in length. In certain embodiments, target DNA sequence is at least 1 kb in length. In certain embodiments, the DNA template is at least 1.5 kb in length.

In certain embodiments, the DNA polymerase is a bacteriophage DNA polymerase. In certain embodiments, the DNA polymerase is derived from Escherichia virus T5, also referred to as bacteriophage T5 (NCBI Accession NO: YP_006950.1). Accordingly, in certain embodiments, the recombinant polypeptide comprises a T5 DNA polymerase amino acid sequence operably linked to a DNA helicase amino acid sequence. In certain embodiments, the recombinant polypeptide comprises a T5 DNA polymerase operably linked to a DNA helicase, wherein the DNA helicase has a slower speed compared to the T5 DNA polymerase. In certain embodiments, the DNA helicase has a speed less than about 150, 160, 170, 180, 190, 200, 210, 220 or 230 bp/s. In certain embodiments, the DNA helicase has a speed less than about 200 bp/s. In certain embodiments, the DNA helicase has a speed less than about 150 bp/s. In certain embodiments, the DNA helicase has a speed less than about 145 bp/s.

In certain embodiments, the T5 DNA polymerase amino acid sequence has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1.

In certain embodiments, the T5 DNA polymerase is encoded by a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:29.

In certain embodiments, the T5 DNA polymerase amino acid sequence comprises SEQ ID NO:1. In certain embodiments, the T5 DNA polymerase amino acid sequence consists of SEQ ID NO:1.

In certain embodiments, the DNA polymerase is an error-prone polymerase. An error-prone DNA polymerase refers to a low fidelity polymerase (e.g., lacks a functional proofreading domain) that has a higher probability of introducing base substitutions or frameshift mutations in a replicated DNA molecule. DNA polymerase error rate (e.g., error per base per replication cycle) can be measured in vitro. Methods for measuring DNA polymerase error rate per base are known in the art (see, e.g., Kunkel, T. A. and Tindall, K. R., Biochemistry, 27, 6008-6013 (1988); Barnes, W. M., Gene, 112, 29-35 (1992)). Certain high-fidelity polymerases may have an error rate per base of only about 10⁻⁶ (about 1 error per million bases). Certain polymerases commonly used in molecular cloning may have an error rate per base of about 3×10⁻⁵ to 3×10⁻⁴ (about 1 error per 33,300 bases to about 1 error per 3,300 bases). In certain embodiments, the error-prone polymerase has an error rate per base of about 5×10⁻⁴ to 10⁻². In certain embodiments, the error-prone polymerase has an error rate per base of about 10⁻³ to 10⁻². In certain embodiments, the error-prone polymerase has an error rate per base of about 5×10⁻⁴ (about 1 error per 2,000 bases). In certain embodiments, the error-prone polymerase has an error rate per base of about 10⁻³ (about 1 error per 1,000 bases). In certain embodiments, the error-prone polymerase has an error rate per base of about 2×10⁻³ (about 1 error per 500 bases). In certain embodiments, the error-prone polymerase has an error rate per base of about 10⁻² (about 1 error per 100 bases). In certain embodiments, the error-prone polymerase has an error rate per base of at least about 5×10⁻⁴ (about 1 error per 2,000 bases). In certain embodiments, the error-prone polymerase has an error rate per base of at least about 10⁻³ (about 1 error per 1,000 bases).

Accordingly, in certain embodiments, an error-prone DNA polymerase-helicase recombinant polypeptide as described herein may have the error rate of the error-prone DNA polymerase domain (e.g., about 5×10⁻⁴ to 10⁻², or 10⁻³ to 10-2).

Certain error-prone DNA polymerases are known in the field and are described herein.

In certain embodiments, an error-prone DNA polymerase comprises one or more mutations within the exonuclease (proofreading) domain of the DNA polymerase. In certain embodiments, an error-prone DNA polymerase lacks a functional exonuclease domain. In certain embodiments, an error-prone DNA polymerase comprises an inactivated exonuclease domain. For example, in certain embodiments, an error-prone T5 DNA polymerase comprises one or more mutations, e.g., one or more mutations selected from the group consisting of D164A, E166A, and 1308V. In certain embodiments, the error-prone T5 DNA polymerase comprises a D164A mutation. In certain embodiments, the error-prone T5 DNA polymerase comprises a E166A mutation. In certain embodiments, the error-prone T5 DNA polymerase comprises a I308V mutation. In certain embodiments, the error-prone T5 DNA polymerase comprises D164A and E166A. In certain embodiments, the error-prone T5 DNA polymerase comprises D164A, E166A and I308V.

In certain embodiments, an error-prone DNA polymerase comprises one or more mutations within the substrate recognition domain. For example, in certain embodiments, an error-prone T5 DNA polymerase comprises a A593R mutation.

In certain embodiments, an error-prone T5 DNA polymerase comprises one or more mutations selected from the group consisting of D164A, E166A, I308V, and A593R. In certain embodiments, the error-prone T5 DNA polymerase comprises D164A, E166A, and A593R. In certain embodiments, the error-prone T5 DNA polymerase comprises D164A, E166A, I308V, and A593R.

In certain embodiments, the T5 DNA polymerase amino acid sequence has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:2. In certain embodiments, the T5 DNA polymerase amino acid sequence has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:3. In certain embodiments, the T5 DNA polymerase amino acid sequence has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:4.

In certain embodiments, the T5 DNA polymerase amino acid sequence comprises SEQ ID NO:2. In certain embodiments, the T5 DNA polymerase amino acid sequence comprises SEQ ID NO:3. In certain embodiments, the T5 DNA polymerase amino acid sequence comprises SEQ ID NO:4. In certain embodiments, the T5 DNA polymerase amino acid sequence consists of SEQ ID NO:2. In certain embodiments, the T5 DNA polymerase amino acid sequence consists of SEQ ID NO:3. In certain embodiments, the T5 DNA polymerase amino acid sequence consists of SEQ ID NO:4.

In certain embodiments, the T5 DNA polymerase is encoded by a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:26.

In certain embodiments, an error-prone DNA polymerase-helicase recombinant polypeptide as described herein confers a mutation rate (e.g., on the target DNA sequence) in the range of about 10²-10⁶, 10³-10⁶, 10⁴-10⁶, or 10⁵-10⁶ fold higher than a background mutation rate (e.g., in a corresponding control cell). For example, the background mutation rate of spontaneous base-pair substitutions (BPSs) of E. coli K12 has been reported to be about 2×10⁻¹⁰ mutations per nucleotide per generation. In certain embodiments, an error-prone DNA polymerase-helicase recombinant polypeptide as described herein confers a mutation rate (e.g., on the target DNA sequence) of about 10⁵-10⁶ fold higher than the background mutation rate. In certain embodiments, an error-prone DNA polymerase-helicase recombinant polypeptide confers a mutation rate (e.g., on the target DNA sequence) at least about 10², 10³, 10⁴, 10⁵, or 10⁶, fold higher than the background mutation rate. In certain embodiments, an error-prone DNA polymerase-helicase recombinant polypeptide confers a mutation rate (e.g., on the target DNA sequence) at least about 10⁵ fold higher than the background mutation rate. In certain embodiments, an error-prone DNA polymerase-helicase recombinant polypeptide confers a mutation rate (e.g., on the target DNA sequence) at least about 2×10⁵ fold higher than the background mutation rate.

In certain embodiments, a recombinant polypeptide described herein is capable of replicating or mutagenizing a DNA (e.g., a nicked DNA).

In certain embodiments, the DNA polymerase is a bacterial DNA polymerase.

Certain embodiments of the invention also provide a T5 DNA polymerase comprising a I308V mutation, wherein the substitution and position are in reference to SEQ ID NO:1. Certain embodiments of the invention provide a recombinant polypeptide that comprises a T5 DNA polymerase comprising a I308V mutation. In certain embodiments, the T5 DNA polymerase comprising a mutation of I308V comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:3 or 4.

### DNA Helicase

In certain embodiments, the DNA helicase is a bacterial DNA helicase. In certain embodiments, the DNA helicase is derived from an Enterobacteriaceae species. In certain embodiments, the DNA helicase is derived from Escherichia co/i. In certain embodiments, the DNA helicase is derived from E. coli K12. In certain embodiments, the DNA helicase is derived from E. coli K12 strain MG1655. In certain embodiments, the DNA helicase is derived from E. coli 042.

In certain embodiments, the DNA helicase is a Rep helicase (see, e.g., NCBI accession number: WP_001238899.1 or SEQ ID NO:5), or a fragment thereof (e.g., catalytically active fragment). For example, the DNA helicase may be a wildtype Rep helicase, or a catalytically active fragment thereof, capable of unwinding DNA. In certain embodiments, the Rep helicase amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:5. In certain embodiments, the Rep helicase amino acid sequence comprises SEQ ID NO: 5. In certain embodiments, the Rep helicase amino acid sequence consists of SEQ ID NO:5.

In certain embodiments, the DNA helicase, or a fragment thereof (e.g., a catalytically active fragment thereof), is a Rep helicase mutant such as a C-terminal truncated Rep helicase (e.g., a Rep helicase that lacks the wild type protein's last 33 amino acids at C-terminal end of the protein).

In certain embodiments, a DNA replisome described herein comprising a Rep helicase mutant (e.g., SEQ ID NO:20) may have a lower off-target effect compared to the wild type Rep helicase (SEQ ID NO:5). In certain embodiments, a Rep helicase mutant (e.g., SEQ ID NO:20) may have a higher affinity for plasmid DNA over chromosomal DNA compared to the wild type Rep helicase (SEQ ID NO:5).

In certain embodiments, the Rep helicase amino acid sequence may have at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:20. In certain embodiments, the Rep helicase amino acid sequence comprises SEQ ID NO:20. In certain embodiments, the Rep helicase amino acid sequence consists of SEQ ID NO:20.

In certain embodiments, the Rep helicase is encoded by a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:27 or 28.

Accordingly, certain embodiments of the invention provide a recombinant polypeptide comprising a DNA polymerase amino acid sequence operably linked to a Rep helicase amino acid sequence. In certain embodiments, the recombinant polypeptide comprises a DNA polymerase amino acid sequence operably linked to a Rep helicase amino acid sequence, wherein the DNA polymerase has a higher speed than the Rep helicase (e.g., a DNA polymerase is faster than about 144 bp/s).

In certain embodiments, the present invention provides a recombinant polypeptide comprising a T5 DNA polymerase amino acid sequence operably linked to a Rep helicase amino acid sequence (e.g., from N-terminus to C-terminus). In certain embodiments, the T5 DNA polymerase has at least about 80% sequence identity to SEQ ID NO:1, 2, 3, or 4, and the Rep helicase amino acid sequence has at least about 80% sequence identity to SEQ ID NO:5. In certain embodiments, the T5 DNA polymerase has at least about 95% sequence identity to SEQ ID NO:1, 2, 3, or 4, and the Rep helicase amino acid sequence has at least about 95% sequence identity to SEQ ID NO:5. In certain embodiments, the T5 DNA polymerase has about 100% sequence identity to SEQ ID NO: 1, 2, 3, or 4, and the Rep helicase amino acid sequence has about 100% sequence identity to SEQ ID NO:5.

In certain embodiments, the T5 DNA polymerase has at least about 80% sequence identity to SEQ ID NO:1, 2, 3, or 4, and the Rep helicase amino acid sequence has at least about 80% sequence identity to SEQ ID NO:20. In certain embodiments, the T5 DNA polymerase has at least about 95% sequence identity to SEQ ID NO: 1, 2, 3, or 4, and the Rep helicase amino acid sequence has at least about 95% sequence identity to SEQ ID NO:20. In certain embodiments, the T5 DNA polymerase has about 100% sequence identity to SEQ ID NO: 1, 2, 3, or 4, and the Rep helicase amino acid sequence has about 100% sequence identity to SEQ ID NO:20.

In certain embodiments, the Rep helicase amino acid sequence in the recombinant polypeptide (e.g., T5-Rep fusion) does not start with a Methionine (M) (see SEQ ID NO: 5 or 20). In certain embodiments, the Rep helicase amino acid sequence in the recombinant polypeptide (e.g., T5-Rep) may start with a Methionine (M) (see Rep helicase of NCBI accession number: WP_001238899.1).

### Operable Linkage

The nature of the linkage between the DNA polymerase amino acid sequence and the DNA helicase amino acid sequence is not critical provided the resulting recombinant polypeptide retains the useful biological properties described herein (e.g., the DNA polymerase amino acid sequence retains its functionality and the DNA helicase amino acid sequence retains its functionality). Therefore, the linking group may be any linkage suitable for joining the two amino acid sequences. In certain embodiments, the DNA polymerase, after fusion with the DNA helicase, has at least about 20%, 30%, 40%, 50%, 60%, 70%, 80%, 90%, 100%, or more of its original DNA polymerization efficiency. In certain embodiments, the DNA polymerase, after fusion with the DNA helicase, maintains its original DNA polymerization efficiency. In certain embodiments, the DNA helicase, after fusion with the DNA polymerase, has at least about 20%, 30%, 40%, 50%, 60%, 70%, 80%, 90%, 100%, or more of its original DNA unwinding efficiency. In certain embodiments, the DNA helicase, after fusion with the DNA polymerase, maintains its original DNA unwinding efficiency.

In certain embodiments, the DNA polymerase amino acid sequence is operably linked to the DNA helicase amino acid sequence via a peptide linker. Thus, in certain embodiments, the recombinant polypeptide described herein further comprises a peptide linker located between the different functional domains of the recombinant polypeptide. In certain embodiments, the peptide linker serves as an inert protein bridge that confers flexibility and/or space for each of the different functional domains to function effectively. For example, the peptide linker may allow diffusion of the two connected proteins along the linker at a rate comparable to the diffusion of free proteins.

In certain embodiments, the peptide linker is about 4 to 300, 10 to 200, 20 to 150, 30 to 100, 40 to 90, or 60 to 85 amino acid (aa) in length. In certain embodiments, the peptide linker is about 4 to 100 aa in length. In certain embodiments, the peptide linker is about 20 to 90 aa in length. In certain embodiments, the peptide linker is about 30 to 80 aa in length. In certain embodiments, the peptide linker is about 20 to 55 aa in length. In certain embodiments, the peptide linker is about 20 to 35 aa in length. In certain embodiments, the peptide linker is about 35 to 85 aa in length. In certain embodiments, the peptide linker is about 80 aa in length. In certain embodiments, the peptide linker is about 81 aa in length.

In certain embodiments, the recombinant polypeptide comprises DNA polymerase and helicase amino acid sequences operably linked in an orientation from N-terminus to C-terminus (the DNA polymerase is located towards the N-terminus of the recombinant polypeptide and the helicase is located towards the C-terminus). In certain embodiments, the peptide linker is linked to the C-terminus of the DNA polymerase amino acid sequence and is linked to the N-terminus of the DNA helicase amino acid sequence.

In certain embodiments, the peptide linker comprises a disordered peptide sequence derived from human alpha-synuclein. In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:6.

In certain embodiments, the peptide linker comprises SEQ ID NO:6. In certain embodiments, the peptide linker consists of SEQ ID NO:6.

In certain embodiments, the peptide linker is a glycine rich peptide linker comprising 4, 5, 6, 7, 8, 9, 10, 11, 12 or more glycine residues, or a glycine serine peptide linker (e.g., GS, GGGGS (SEQ ID NO: 39), (GGGGS)₃ (SEQ ID NO: 40) or (GGGGS)₄) (SEQ ID NO: 7). In certain embodiments, the peptide linker comprises 4 continuous glycine residues (SEQ ID NO: 41). In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:7 or 8. In certain embodiments, the peptide linker comprises SEQ ID NO:7 or 8.

In certain embodiments, the peptide linker comprises both a glycine rich sequence and another disordered peptide sequence (e.g., derived from alpha-synuclein). In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:9. In certain embodiments, the peptide linker comprises SEQ ID NO:9. In certain embodiments, the peptide linker consists of SEQ ID NO:9.

### Certain Recombinant Polypeptide Embodiments

In certain embodiments, a recombinant polypeptide described herein comprises a T5 DNA polymerase amino acid sequence operably linked to a Rep helicase amino acid sequence via a peptide linker.

In certain embodiments, the peptide linker comprises a disordered peptide sequence derived from human alpha-synuclein. In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:6. In certain embodiments, the peptide linker comprises SEQ ID NO:6. In certain embodiments, the peptide linker consists of SEQ ID NO:6.

In certain embodiments, the peptide linker is a glycine rich peptide linker comprising 4, 5, 6, 7, 8, 9, 10, 11, 12 or more glycine residues, or a glycine serine peptide linker (e.g., GS, GGGGS (SEQ ID NO: 39), (GGGGS)₃ (SEQ ID NO: 40) or (GGGGS)₄) (SEQ ID NO: 7). In certain embodiments, the peptide linker comprises 4 continuous glycine residues (SEQ ID NO: 41). In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:7 or 8. In certain embodiments, the peptide linker comprises SEQ ID NO:7 or 8.

In certain embodiments, the peptide linker comprises both a glycine rich sequence and another disordered peptide sequence (e.g., derived from alpha-synuclein). In certain embodiments, the peptide linker amino acid sequence has as at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:9. In certain embodiments, the peptide linker comprises SEQ ID NO:9. In certain embodiments, the peptide linker consists of SEQ ID NO:9.

In certain embodiments, the recombinant polypeptide comprises T5 DNA polymerase and Rep helicase amino acid sequences operably linked in an orientation from N-terminus to C-terminus (T5 DNA polymerase is located towards the N-terminus of the recombinant polypeptide and the Rep helicase is located towards the C-terminus).

In certain embodiments, the recombinant polypeptide comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:10, 11, 22, or 23. In certain embodiments, the recombinant polypeptide consists of an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 10, 11, 22, or 23.

In certain embodiments, the recombinant polypeptide comprises (or consists of) an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:10. In certain embodiments, the recombinant polypeptide comprises (or consists of) SEQ ID NO:10. In certain embodiments, the recombinant polypeptide comprises (or consists of) an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:11. In certain embodiments, the recombinant polypeptide comprises (or consists of) SEQ ID NO:11. In certain embodiments, the recombinant polypeptide comprises (or consists of) an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:22. In certain embodiments, the recombinant polypeptide comprises (or consists of) SEQ ID NO:22. In certain embodiments, the recombinant polypeptide comprises (or consists of) an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:23. In certain embodiments, the recombinant polypeptide comprises (or consists of) SEQ ID NO:23.

In certain embodiments, the recombinant polypeptide is encoded by a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:12.

### Nucleic Acids, Expression Cassettes and Vectors Encoding a Recombinant Polypeptide

Certain embodiments provide a nucleic acid comprising a sequence encoding a recombinant polypeptide as described herein. In certain embodiments, the nucleic acid is codon-optimized for efficient expression in a host cell.

Certain embodiments also provide an expression cassette comprising a nucleic acid as described herein (e.g., encoding a recombinant polypeptide as described herein). In certain embodiments, the expression cassette comprises a bacteriophage promoter. In certain embodiments, the expression cassette comprises a T7 promoter. In certain embodiments, the expression cassette comprises a T5 promoter. In certain embodiments, the expression cassette comprises a bacterial promoter. In certain embodiments, the expression cassette further comprises a terminator sequence (e.g., T7 terminator sequence).

In certain embodiments, the expression cassette further comprises an engineered 5′-untranslated region (5′ UTR) that is downstream of the promoter but upstream of the nucleic acid sequence encoding the recombinant polypeptide. For example, the engineered 5′ UTR may provide tighter binding of the ribosome to the translation initiation site in the 5′ UTR of a transcript. In certain embodiments, the 5′UTR comprises a ribosomal binding site sequence (e.g., TTGAGGT). In certain embodiments, the expression cassette comprises a 5′-untranslated region (5′ UTR) having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:16. In certain embodiments, the engineered 5′ UTR can form a stem-loop structure.

Certain embodiments of the invention also provide a vector comprising a nucleic acid encoding a recombinant polypeptide as described herein or an expression cassette comprising such a nucleic acid. The term “helper vector” may be used to reference such a vector. The helper vector may be introduced into a host cell to express a DNA polymerase-helicase recombinant polypeptide as described herein. In certain embodiments, the vector is a plasmid, phagemid or phage vector as non-limiting examples.

Certain embodiments also provide a cell (e.g., a host cell) comprising a nucleic acid comprising a sequence encoding a recombinant polypeptide as described herein, an expression cassette comprising such a nucleic acid, or a helper vector as described herein. In certain embodiments, the nucleic acid or expression cassette as described herein is comprised in a chromosomal DNA or plasmid DNA.

### Targeted Artificial DNA Replisome (TADR) Complex

As described herein, a recombinant polypeptide of the invention may be used to replicate and/or mutagenize a target double stranded DNA (dsDNA) sequence (e.g., comprising a DNA nickase initiation sequence operably linked upstream of a target DNA sequence). A corresponding DNA nickase that recognizes the initiation sequence may be used to nick one strand of the target dsDNA and recruit the recombinant polypeptide to form a DNA replication complex. The helicase portion of the recombinant polypeptide can then unwind the dsDNA from the nick site and the DNA polymerase portion is then able to add nucleotides to the free hydroxyl group to synthesize a new strand.

Accordingly, certain embodiments provide a DNA replisome complex comprising: 1) a target dsDNA comprising a DNA nickase initiation sequence operably linked upstream of a target DNA sequence; 2) a corresponding DNA nickase, which is capable of nicking the dsDNA at the initiation sequence; and 3) a recombinant polypeptide as described herein, wherein the recombinant polypeptide is capable of replicating the nicked dsDNA.

Certain embodiments of the invention also provide a DNA-protein complex comprising: 1) a nicked target dsDNA as described herein comprising a DNA nickase initiation sequence operably linked upstream of a target DNA sequence; and 2) a recombinant polypeptide as described herein, wherein the recombinant polypeptide is capable of replicating the nicked dsDNA.

In certain embodiments, the DNA replisome complex is capable of assembling within a cell in vivo. In certain embodiments, the DNA replisome complex is capable of assembling in a cell-free manner in vitro (e.g., within a PCR tube or test tube).

In certain embodiments, a targeted DNA replisome complex as described herein confers a targeted mutation rate (e.g., within the target sequence) in the range of about 10²-10⁶, 10³-10⁶, 10⁴-10⁶, or 10⁵-10⁶ fold higher than the background mutation rate. In certain embodiments, a targeted DNA replication complex as described herein confers a targeted mutation rate of about 10⁵-10⁶ fold higher than the background mutation rate. In certain embodiments, a targeted DNA replisome complex as described herein confers a targeted mutation rate at least about 10², 10³, 104, 105, or 10⁶ fold higher than the background mutation rate. In certain embodiments, a targeted DNA replisome complex as described herein confers a targeted mutation rate at least about 10⁵ fold higher than the background mutation rate. In certain embodiments, a targeted DNA replisome complex as described herein confers a targeted mutation rate at least about 2×10⁵ fold higher than the background mutation rate.

In certain embodiments, a targeted DNA replisome complex as described herein confers an off-target mutation rate (e.g., outside the targeted sequence) in the range of no more than about 1-100, 5-90, or 40-80 fold higher than the background mutation rate. In certain embodiments, a targeted DNA replisome complex as described herein confers an off-target mutation rate less than about 1, 5, 10, 20, 30, 40, 50, 60, 70, 80, or 90-fold of the background mutation rate. In certain embodiments, a targeted DNA replication complex as described herein confers an off-target mutation rate less than about 78-fold of the background mutation rate.

### DNA Nickase

As described herein, a DNA nickase, or a catalytically active fragment thereof, may be used in combination with a corresponding initiation sequence to confer specificity/selectivity to the recruitment of a recombinant polypeptide as described herein, which is used in turn for the replication or mutagenesis of a target dsDNA.

In certain embodiments, the DNA nickase is derived from a bacteriophage. In certain embodiments, the DNA nickase is derived from bacteriophage PhiX 174. In certain embodiments, the DNA nickase is CisA (NCBI accession number P03631.1). Accordingly, in certain embodiments, the DNA nickase has an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:24. In certain embodiments, the DNA nickase has one or more mutations relative to CisA (NCBI accession number P03631.1; SEQ ID NO:24). For example, the DNA nickase may be a CisA mutant that comprises point mutation Y303H (SEQ ID NO:30) relative to CisA (NCBI accession number P03631.1; SEQ ID NO:24). Accordingly, in certain embodiments, the DNA nickase has an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:30. In certain embodiments, the DNA nickase is encoded by a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:25. In certain embodiments, the DNA nickase is encoded by a nucleic acid sequence having at least about 95%, 96%, 97%, 98%, 99% or 100% sequence identity to SEQ ID NO:25. In certain embodiments, the DNA nickase is encoded by a nucleic acid sequence comprising a sequence of SEQ ID NO:25.

In certain embodiments, a nucleic acid encoding a DNA nickase, or a catalytically active fragment thereof, is used to express the DNA nickase in a host cell (e.g., for use in generating a DNA replication complex or for use in a method described herein). In certain embodiments, the nucleic acid is codon-optimized for efficient expression in a host cell. In certain embodiments, such a nucleic acid is comprised within an expression cassette. In certain embodiments, such a nucleic acid or expression cassette is present within a vector, such as a plasmid, phagemid or phage vector that can transform or transfect a host cell for the expression of a DNA nickase.

In certain embodiments, the DNA nickase, or a catalytically active fragment thereof, is transiently expressed by a host cell. In certain embodiments, the DNA nickase, or a catalytically active fragment thereof, is stably expressed by a host cell (i.e. integrated into the genome). In certain embodiments, the DNA nickase, or a catalytically active fragment thereof, can be introduced into a host cell via a helper vector. In certain embodiments, the host cell may constitutively or inducibly express the DNA nickase, or a catalytically active fragment thereof.

In certain embodiments, inducible expression of the DNA nickase (e.g., CisA) can be modulated with carbon source regulatable operon control. For example, the DNA nickase gene may be placed under a T7 promotor, while the T7 RNA polymerase expression is controlled by pBAD promoter. Thus, expression of DNA nickase can be turned off by a suppressive molecule (e.g., glucose). Likewise, expression can be turned on in a suitable condition (e.g., in the presence of glycerol, and/or in the absence of glucose).

Target Double Stranded DNA (dsDNA)

As described herein, a target DNA sequence to be replicated or mutagenized may be included in a target dsDNA, which comprises a DNA nickase initiation sequence. The DNA nickase initiation sequence should correspond to the selected DNA nickase, thereby providing specificity to the replication/mutagenesis. For example, if a CisA DNA nickase is selected, then a corresponding initiation sequence that is recognized by CisA would be included in the target dsDNA. Thus, in certain embodiments, the target dsDNA comprises a DNA nickase initiation sequence. In certain embodiments, a target dsDNA further comprises a target DNA sequence that is operably linked downstream of the DNA nickase initiation sequence.

In certain embodiments, the target dsDNA comprises a DNA nickase initiation sequence, that is targeted by CisA. In certain embodiments, the CisA initiation sequence comprises a nucleic acid sequence having sequence identity of at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:13. In certain embodiments, the CisA initiation sequence comprises SEQ ID NO:13. In certain embodiments, the CisA initiation sequence consists of SEQ ID NO:13. In certain embodiments, the initiation sequence is about 10-50 bp, or 20˜40 bp in length. In certain embodiments, the initiation sequence is about 30 bp in length.

In certain embodiments, the dsDNA further comprises a termination sequence. One or more components of the DNA replisome complex may dissociate at the termination sequence to stop the DNA replication or mutagenesis process. In certain embodiments, the termination sequence is recognized by CisA. In certain embodiments, the termination sequence comprises a nucleic acid sequence having a sequence identity of at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:14 or 21. In certain embodiments, the CisA termination sequence comprises SEQ ID NO:14 or 21. In certain embodiments, the CisA termination sequence consists of SEQ ID NO:14 or 21. In certain embodiments, the termination sequence is about 10-50 bp, 20-35 bp, or 20˜40 bp in length. In certain embodiments, the termination sequence is about 24, 25, 26, 27, 28, 29, 30 or more bp in length. In certain embodiments, the termination sequence is about 30 bp in length. In certain embodiments, the termination sequence is about 24 bp in length. In certain embodiments, the dsDNA may comprise two or more termination sequences to enhance termination efficiency. In certain embodiments, the dsDNA comprises two, three, four or more copies of the termination sequences (e.g., SEQ ID NO:14 or 21). For example, a plurality of termination sequence may be operably linked together (e.g., in tandem) to enhance termination efficiency.

In certain embodiments, the target DNA sequence comprises one or more genes, or a fragment thereof. In certain embodiments, the target DNA sequence comprises one gene. In certain embodiments, the target DNA sequence comprises two genes. In certain embodiments, the target DNA sequence comprises three genes. In certain embodiments, the target DNA sequence comprises four genes. In certain embodiments, the target DNA sequence comprises five genes. In certain embodiments, the target DNA sequence comprises a plurality of genes (e.g., 2 or more genes). Hence, a targeted DNA replication complex described herein is capable of replicating or mutagenizing the target DNA sequence (e.g., a gene or a gene cluster as a mutagenesis target). In certain embodiments, the target DNA sequence (e.g., a gene or a plurality of genes) downstream of the initiation sequence is upstream of the termination sequence.

In certain embodiments, the target DNA sequence is a cDNA sequence.

In certain embodiments, the target DNA sequence may encode a protein or a non-coding RNA molecule. In certain other embodiments, the target DNA sequence is a non-coding regulatory element.

Thus, certain embodiments provide a nucleic acid sequence comprising a target dsDNA as described herein, or an expression cassette comprising such a nucleic acid sequence as described herein. Certain embodiments also provide a nicked target dsDNA as described herein.

Certain embodiments of the invention also provide a vector comprising a nucleic acid comprising a target dsDNA or an expression cassette comprising such a nucleic acid. The term “target vector” may be used to reference such a vector. The target vector may be introduced into a host cell. In certain embodiments, the vector is a plasmid, phagemid or phage vector as non-limiting examples.

Certain embodiments also provide a cell (e.g., a host cell) comprising a nucleic acid comprising a target dsDNA as described herein, an expression cassette comprising such a nucleic acid, or a target vector as described herein.

### Host Cells

Certain embodiments of the invention provide a cell (e.g., a host cell) comprising at least one polypeptide described herein, at least one nucleic acid as described herein, at least one expression cassette as described herein and/or at least one vector described herein. In certain embodiments, the cell is a prokaryotic cell (e.g., a bacterial cell). In certain embodiments, the cell is E. coli cell.

Certain embodiments of the invention provide a cell (e.g., a host cell) comprising a DNA replication complex as described herein, a helper vector, a vector encoding a DNA nickase, and/or a target vector as described herein.

Certain embodiments of the invention provide a cell (e.g., a host cell) comprising a DNA replication complex as described herein, a helper vector, a stably integrated DNA nickase, and/or a target vector as described herein.

Certain embodiments of the invention provide a cell (e.g., a host cell) comprising a DNA replication complex as described herein.

Certain embodiments also provide a cell (e.g., a host cell) comprising a nucleic acid comprising a sequence encoding a recombinant polypeptide as described herein (e.g., T5-Rep fusion protein) and/or a DNA nickase (e.g., CisA), an expression cassette comprising such a nucleic acid, and/or a helper vector as described herein.

In certain embodiments, the host cell lacks native wild type Rep helicase (e.g., the original endogenous Rep helicase of a host cell has been deleted). Accordingly, a host cell may only have Rep helicase in the format of the recombinant polypeptide as described herein (e.g., T5-Rep fusion protein).

In certain embodiments, the cell's chromosomal DNA comprises a sequence encoding a recombinant polypeptide as described herein (e.g., T5-Rep fusion protein) and/or a DNA nickase (e.g., CisA). In certain embodiments, the cell comprises a plasmid comprising a sequence encoding a recombinant polypeptide as described herein (e.g., T5-Rep fusion protein) and/or a DNA nickase (e.g., CisA). For example, the cell may comprise 1) a chromosomal or plasmid gene for a T5-Rep fusion protein, and 2) a chromosomal or plasmid gene for CisA.

In certain embodiments, the cell comprises a chromosomal gene for a T5-Rep fusion protein and a chromosomal gene for CisA. In certain embodiments, the cell comprises a plasmid gene for a T5-Rep fusion protein and a plasmid gene for CisA. In certain embodiments, the cell comprises a plasmid gene for a T5-Rep fusion protein and a chromosomal gene for CisA. In certain embodiments, the cell comprises a chromosomal gene for a T5-Rep fusion protein and a plasmid gene for CisA.

In certain embodiments, the cell comprises a helper vector comprising a sequence encoding a recombinant polypeptide as described herein (e.g., T5-Rep fusion protein) and/or a DNA nickase (e.g., CisA).

In certain embodiments, the cell does not comprise a helper vector, for example, both the recombinant polypeptide as described herein (e.g., T5-Rep fusion protein) and the DNA nickase (e.g., CisA) are encoded by chromosomal genes of the cell (e.g., E. coli cell).

In certain embodiments, the DNA nickase, or a catalytically active fragment thereof, is transiently expressed by a host cell. In certain embodiments, the DNA nickase, or a catalytically active fragment thereof, is stably expressed by a host cell (i.e. integrated into the genome). In certain embodiments, the DNA nickase, or a catalytically active fragment thereof, can be introduced into a host cell via a helper vector. In certain embodiments, the host cell may constitutively or inducibly express the DNA nickase, or a catalytically active fragment thereof.

In certain embodiments, the recombinant polypeptide described herein (e.g., T5-Rep fusion protein) is transiently expressed by a host cell. In certain embodiments, the recombinant polypeptide described herein (e.g., T5-Rep fusion protein) is stably expressed by a host cell (i.e. integrated into the genome). In certain embodiments, the recombinant polypeptide described herein (e.g., T5-Rep fusion protein) can be introduced into a host cell via a helper vector. In certain embodiments, the host cell may constitutively or inducibly express the recombinant polypeptide described herein (e.g., T5-Rep fusion protein).

In certain embodiments, inducible expression of the DNA nickase (e.g., CisA) and/or a recombinant polypeptide described herein (e.g., T5-Rep) can be modulated with carbon source regulatable operon control. For example, the DNA nickase gene and/or a recombinant polypeptide described herein might be placed under a T7 promotor, while the T7 RNA polymerase expression is controlled by pBAD promoter. Thus, expression of DNA nickase and/or a recombinant polypeptide described herein can be turned off by a suppressive molecule (e.g., glucose). Likewise, expression can be turned on in a suitable condition (e.g., in the presence of glycerol, and/or in the absence of glucose).

### Targeted Mutagenesis System and Kits

As described herein, the DNA nickase recognizes a DNA nickase initiation sequence and initiates assembly of the artificial DNA replisome complex as described, conferring specificity/selectivity to the recruitment of the recombinant polypeptide for mutagenesis of the target dsDNA.

Accordingly, certain embodiments of the invention provide a targeted DNA replication or mutagenesis system, the components of which comprise:
1) a target dsDNA comprising a DNA nickase initiation sequence, and optionally, a target DNA sequence operably linked downstream of the initiation sequence; or a target vector as described herein;
2) a DNA nickase; a vector comprising a nucleic acid sequence encoding a DNA nickase or an expression cassette thereof, or a host cell comprising the vector or DNA nickase; and
3) a recombinant polypeptide or a helper vector as described herein;
wherein the DNA nickase is capable of nicking the target dsDNA/the target vector at the DNA nickase initiation sequence; and wherein the recombinant polypeptide is capable of replicating or mutagenizing the nicked target dsDNA/target vector.

In certain embodiments, the targeted DNA replication or mutagenesis system comprises:
1) a target vector as described herein;
2) a host cell comprising the DNA nickase (e.g., CisA); and
3) a helper vector as described herein;
wherein the DNA nickase is capable of nicking the target vector at the DNA nickase initiation sequence; and wherein the recombinant polypeptide, expressed from the helper vector, is capable of replicating or mutagenizing the nicked target vector.

In certain embodiments, the targeted DNA replication or mutagenesis system comprises:
1) a target vector as described herein; and
2) a host cell comprising the DNA nickase (e.g., CisA) and the recombinant polypeptide as described herein;
wherein the DNA nickase is capable of nicking the target vector at the DNA nickase 30 initiation sequence; and wherein the recombinant polypeptide is capable of replicating or mutagenizing the nicked target vector. As a non-limiting example, the host cell may comprise chromosomal genes encoding the DNA nickase (e.g., CisA) and the recombinant polypeptide as described herein (e.g., T5-Rep fusion).

In certain embodiments, the targeted DNA replication or mutagenesis system comprises:
1) a target vector as described herein;
2) a host cell; and
3) a helper vector as described herein;
wherein the DNA nickase, expressed from the helper vector, is capable of nicking the target vector at the DNA nickase initiation sequence; and wherein the recombinant polypeptide, expressed from the helper vector, is capable of replicating or mutagenizing the nicked target vector.

In certain embodiments, the present invention provides a cell comprising a polypeptide described herein (e.g., a DNA polymerase-helicase recombinant polypeptide or a DNA nickase), a nucleic acid described herein, and/or a vector described herein. The recombinant polypeptide/other components of the targeted mutagenesis system can be introduced via delivery/expression of a vector comprising a nucleic acid as described herein. In certain embodiments, the present invention provides a cell that has been transformed (e.g., transfected or transduced) by one or more vectors described herein. In certain embodiments, the cell is a prokaryotic cell (e.g., a bacterial cell). In certain embodiments, the cell is E. coli cell.

Certain embodiments of the invention provide a host cell comprising the helper vector as described herein. Certain embodiments of the invention provide a host cell comprising the target vector as described herein. Certain embodiments of the invention provide a host cell comprising the vector comprising a nucleic acid sequence encoding a DNA nickase or an expression cassette as described herein.

Certain embodiments of the invention provide a host cell comprising the helper vector and the target vector. Certain embodiments of the invention provide a host cell comprising a DNA nickase (e.g., CisA), the helper vector, and/or the target vector. Certain embodiments of the invention provide a host cell comprising the vector comprising a nucleic acid sequence encoding a DNA nickase or an expression cassette as described herein, the helper vector, and/or the target vector.

The targeted DNA mutagenesis system described herein may be used to replicate or mutagenize one or more genes within a cell. Alternatively, the system may be used to manipulate genetic sequences that are not present in a cell (e.g., in a PCR tube or test tube).

Certain embodiments provide a kit comprising:
1) a nucleic acid encoding a recombinant polypeptide as described herein; and
2) instructions for replicating/mutagenizing a target dsDNA sequence (e.g., using a method as described herein).

In certain embodiments, the nucleic acid is present in a vector. Thus, in certain embodiments, the kit comprises a helper vector as described herein. In certain embodiments, the kit comprises a host cell comprising the helper vector.

In certain embodiments, the kit further comprises a nucleic acid encoding a DNA nickase. In certain embodiments, the nucleic acid is present in a vector. In certain embodiments, the kit further comprises a host cell comprising the DNA nickase vector. In certain embodiments, the kit comprises a host cell that stably expresses a DNA nickase.

In certain embodiments, the kit further comprises a target dsDNA comprising an DNA nickase initiation sequence, and optionally a DNA nickase termination sequence downstream of the initiation sequence. In certain embodiments, the target dsDNA is present in a vector. In certain embodiments, the target dsDNA further comprises a target DNA sequence downstream of the DNA nickase initiation sequence (i.e., a target dsDNA). In certain embodiments, the kit further comprises instructions for inserting a target DNA sequence of interest downstream of the DNA nickase initiation sequence. For example, methods for inserting a target sequence of interest into a target vector are known in the art and described herein. Non-limiting exemplary methods may include restriction enzyme-based digestion and ligation or any other suitable molecular cloning technique to insert a sequence of interest into a target vector. In certain embodiments, the target vector is prelinearized and/or may have a terminal overhang to facilitate easy insertion of a target sequence of interest. In certain embodiments, the target dsDNA is present within a cell.

Certain embodiments provide a kit comprising one or more vectors as described herein (e.g., a target vector and/or a helper vector) and instructions for replicating/mutagenizing a target dsDNA sequence (e.g., using a method as described herein).

Certain embodiments provide a kit comprising one or more cells as described herein and instructions for replicating/mutagenizing a target dsDNA sequence (e.g., using a method as described herein).

Certain embodiments provide a kit comprising one or more cells as described herein and one or more vectors as described herein and instructions for replicating/mutagenizing a target dsDNA sequence (e.g., using a method as described herein).

Certain embodiments provide a kit comprising a target vector and instructions for inserting a target DNA sequence into the target vector (e.g., downstream of a DNA nickase initiation sequence, and upstream of a termination sequence) and contacting the target vector with a DNA nickase, a DNA helicase and/or an error-prone DNA polymerase (e.g., a recombinant polypeptide as described herein).

Certain embodiments provide a kit comprising:
1) a target vector comprising a DNA nickase initiation sequence;
2) a host cell comprising a DNA nickase (e.g., CisA);
3) a helper vector as described herein;
4) packaging materials; and
5) instructions for inserting a target DNA sequence into the target vector downstream of the DNA nickase initiation sequence; and contacting the host cell with the helper vector and the target vector comprising the target DNA sequence under conditions suitable for the vectors to enter the host cell and mutagenize the target vector.

Certain embodiments provide a kit comprising:
1) a target vector comprising a DNA nickase initiation sequence;
2) a host cell comprising a DNA nickase (e.g., CisA) and a recombinant polypeptide as described herein (e.g., T5-rep fusion protein);
3) packaging materials; and
4) instructions for inserting a target DNA sequence into the target vector downstream of the DNA nickase initiation sequence; and contacting the host cell with the target vector comprising the target DNA sequence under conditions suitable for the vector to enter the host cell and mutagenize the target vector.

In certain embodiments, the host cell comprises chromosomal DNA comprising a nucleic acid sequence encoding the DNA nickase (e.g., CisA) and a nucleic acid sequence encoding the recombinant polypeptide as described herein (e.g., T5-rep fusion protein).

### Certain DNA Replication or Mutagenesis Methods

Certain embodiments provide a method comprising contacting a cell with a nucleic acid encoding a recombinant polypeptide as described herein. In certain embodiments, the nucleic acid is present in a vector, such as a helper vector as described herein. In certain embodiments, the nucleic acid is present in chromosomal DNA of the cell. In certain embodiments, the cell expresses a DNA nickase (e.g., stably expresses a DNA nickase). In certain embodiments, the method further comprises contacting the cell with a nucleic acid encoding a DNA nickase (e.g., the nucleic acid is present within a vector). In certain embodiments, the method further comprises contacting the cell with a target dsDNA (e.g., a target vector as described herein), wherein the target dsDNA comprises an DNA nickase initiation sequence operably linked upstream of a target DNA sequence, and optionally, a DNA nickase termination sequence operably linked downstream of the target DNA sequence.

Certain embodiments of the present invention also provide a targeted mutagenesis method that comprises contacting a dsDNA with a DNA nickase, and contacting the nicked dsDNA with a DNA helicase and an error-prone DNA polymerase. In certain embodiments, the DNA helicase and the error-prone DNA polymerase are operably linked to form a recombinant polypeptide.

Certain embodiments of the present invention provide a targeted mutagenesis method that comprises inserting a target DNA sequence into a target dsDNA comprising a DNA nickase initiation sequence, wherein the target DNA sequence is operably linked downstream of the initiation sequence; contacting the target dsDNA with a corresponding DNA nickase that recognizes and nicks the initiation sequence; and contacting the nicked dsDNA with a DNA helicase and an error-prone DNA polymerase. In certain embodiments, the DNA helicase and the error-prone DNA polymerase are operably linked to form a recombinant polypeptide.

Certain embodiments provide a DNA replication or mutagenesis method comprising contacting a target dsDNA with a recombinant polypeptide described herein.

For methods described herein, in certain embodiments, the dsDNA is contacted in a cell. In certain embodiments, the dsDNA is contacted in a cell-free manner (e.g., in a test tube).

Certain embodiments provide a DNA replication or mutagenesis method, which comprises forming a DNA replisome complex as described herein. For example, in certain embodiments, such a method comprises introducing into a cell one or more components of the targeted DNA replication or mutagenesis system as described herein. In certain embodiments, such a method that comprises introducing into a cell a recombinant polypeptide described herein (e.g., via a helper vector). In certain embodiments, such a method comprises introducing into a cell a target vector as described herein. In certain embodiments, such a method comprises introducing into a cell a DNA nickase as described herein (e.g., via a vector or chromosomal DNA integration).

Certain embodiments also provide a method of mutagenizing a target DNA sequence, the method comprising inserting a target DNA sequence into a target dsDNA comprising a DNA nickase initiation sequence, wherein the target DNA sequence is operably linked downstream of the initiation sequence; and assembling a DNA replisome complex as described herein. In certain embodiments, the target DNA sequence is comprised within the target vector as described herein.

For methods described herein, in certain embodiments, the DNA replisome complex is formed or assembled in a cell. In certain embodiments, the DNA replisome complex is formed or assembled in a cell-free manner (e.g., in a test tube).

Certain embodiments also provide a method of mutagenizing a target DNA sequence, the method comprising inserting the target DNA sequence into a target dsDNA comprising a DNA nickase initiation sequence, wherein the target DNA sequence is operably linked downstream of the initiation sequence; and contacting the target DNA sequence with a DNA nickase, a DNA helicase and/or an error-prone DNA polymerase. In certain embodiments, the DNA helicase and the error-prone DNA polymerase are operably linked to form the recombinant polypeptide as described herein. In certain embodiments, the target DNA sequence is comprised within the target vector as described herein. In certain embodiments, the recombinant polypeptide is expressed from a helper vector as described herein. In certain embodiments, the recombinant polypeptide is expressed from chromosomal DNA of the host cell. In certain embodiments, the DNA nickase is CisA. In certain embodiments, the target DNA sequence is operably linked upstream of a termination sequence as described herein.

Certain embodiments provide a method of mutagenizing a target DNA sequence in a cell comprising contacting the target DNA sequence with a recombinant polypeptide as described herein, wherein the target DNA sequence is operably linked downstream of a DNA nickase initiation sequence; and wherein the cell expresses a corresponding DNA nickase; under conditions suitable for the DNA nickase to nick the initiation sequence and for the recombinant polypeptide to mutagenize the target DNA sequence. In certain embodiments, the recombinant polypeptide and/or the DNA nickase are expressed from chromosomal DNA of the host cell. For example, in certain embodiments, the cell comprises chromosomal DNA comprising a nucleic acid sequence encoding a recombinant polypeptide as described herein (e.g., T5-Rep fusion). In certain other embodiments, the method further comprises contacting the cell with a nucleic acid encoding the recombinant polypeptide, or an expression cassette or vector comprising such a nucleic acid (e.g., a helper vector), under conditions suitable to express the recombinant polypeptide. In certain embodiments, the method further comprises contacting the cell with a nucleic acid encoding a DNA nickase (e.g., a nucleic acid is present within a vector), under conditions suitable to express the DNA nickase. In certain embodiments, the method further comprises contacting the cell with a target vector comprising the target DNA sequence operably linked downstream of the DNA nickase initiation sequence.

Certain embodiments also provide a method of mutagenizing a target DNA sequence comprising contacting a host cell that expresses a DNA nickase with: 1) a target vector comprising a corresponding DNA nickase initiation sequence operably linked to the target DNA sequence; and 2) a helper vector as described herein; under conditions suitable for the vectors to enter the host cell; for the DNA nickase to nick the initiation sequence; and for the recombinant polypeptide to replicate or mutagenize the target DNA sequence.

Certain embodiments provide a method of mutagenizing a target DNA sequence comprising:
inserting a target DNA sequence into a target vector as described herein; and
introducing the target vector and a helper vector as described herein into a host cell comprising a DNA nickase (e.g., CisA);
wherein the DNA nickase nicks the target vector and the recombinant polypeptide, expressed from the helper vector, replicates or mutagenizes the target DNA sequence.

Certain embodiments provide a method of mutagenizing a target DNA sequence comprising:
inserting a target DNA sequence into a target vector as described herein; and
introducing the target vector as described herein into a host cell comprising a DNA nickase (e.g., CisA) and a recombinant polypeptide (e.g., T5-Rep fusion);
wherein the DNA nickase nicks the target vector and the recombinant polypeptide, expressed from chromosomal DNA of the host cell, replicates or mutagenizes the target DNA sequence.

Thus, certain embodiments of the invention provide a method of replicating or mutagenizing a target DNA sequence in a cell comprising contacting the cell with one or more components of the DNA mutagenesis system described herein under conditions suitable for the component(s) of the system to enter the cell and mutagenize the target sequence. In certain embodiments, the components of the system (e.g., the helper vector and the target vector) can be introduced to a cell concurrently or sequentially.

Certain embodiments of the methods described herein may introduce from about 1 to 20, 1 to 15, 1 to 10, 1 to 9, 1 to 8, 1 to 7, 1 to 6, 1 to 5, 1 to 4, 1 to 3, or 1 to 2 mutations in a target DNA sequence (e.g., a gene) per mutagenesis round. In certain embodiments, the methods described herein may introduce about 1, 2, 3, 4, 5, 6, 7, 8, 9, 10, 15 or 20 mutations in a target DNA sequence per mutagenesis round. In certain embodiments, the methods described herein may introduce one or more mutation (e.g., substitution) in a target DNA sequence (e.g., a gene) per mutagenesis round. In certain embodiments, the methods described herein may introduce a plurality of mutations (e.g., two, three or four substitutions) in a target DNA sequence (e.g., a gene) per mutagenesis round.

Methods described herein may further comprises culturing the cell under conditions suitable for suppressing (e.g., turn off) TADR mediated mutagenesis, or the expression of a DNA nickase and/or a recombinant polypeptide described herein (e.g., CisA and/or T5-Rep). In certain embodiments, the cell is cultured in the presence of glucose (e.g., at a glucose concentration sufficient to suppress TADR mediated mutagenesis, or the expression of CisA and/or T5-Rep).

Methods described herein may further comprises culturing the cell under conditions suitable for promoting (e.g., turn on) TADR mediated mutagenesis, or the expression of a DNA nickase and/or a recombinant polypeptide described herein (e.g., CisA and/or T5-Rep). In certain embodiments, the cell is cultured in the presence of glycerol and/or in the absence of glucose.

Methods described herein may further comprises culturing the cell under conditions suitable for reducing innate replisome number and/or cell chromosome DNA copy number to reduce off-target mutagenesis. In certain embodiments, the cell is cultured in low nutrient medium or minimal medium to promote on-target mutagenesis and high selectivity and/or suppress off-target mutagenesis. For example, a liquid LB broth (10 g NaCl₂, 10 g tryptone, 5 g yeast extract per liter; Fisher BioReagents) or its agar plates (15 g agar per liter) is considered a high nutrient or rich medium. In comparison, a medium having less than 5 g tryptone and less than 3 g yeast extract or yeast synthetic drop-out medium supplements per liter is considered a low nutrient medium or minimum medium (e.g., 2 g yeast synthetic drop-out medium supplements (Sigma) per liter). In certain embodiments, a low nutrient medium or minimal medium has less than 5 g, 4 g, 3 g, 2 g, or lg tryptone, and less than 3 g, 2.5 g, 2 g, 1.5 g or lg yeast extract or yeast synthetic drop-out medium supplements per liter. The term “yeast synthetic drop-out medium supplements” refers to yeast synthetic drop-out medium supplements without histidine, leucine, tryptophan and uracil, see MilliporeSigma catalog number Y2001. In certain embodiments, the minimal medium has a formula of (1 g (NH₄)₂SO₄, 7 g K₂HPO₄, 2 g KH₂PO₄, 0.1 g MgSO₄, 0.5 g sodium citrate, 4 g glycerol, 2 g yeast synthetic drop-out medium supplements (Sigma)) per liter.

### Certain Embodiments

Embodiment 1. A recombinant polypeptide comprising a T5 DNA polymerase amino acid sequence operably linked to a DNA helicase amino acid sequence.

Embodiment 2. The recombinant polypeptide of embodiment 1, wherein the T5 DNA polymerase is an error-prone polymerase.

Embodiment 3. The recombinant polypeptide according to embodiment 1 or 2, wherein the T5 DNA polymerase comprises one or more mutations selected from the group consisting of D164A, E166A, 1308V, and A593R.

Embodiment 4. The recombinant polypeptide according to any one of embodiments 1-3, wherein the T5 DNA polymerase amino acid sequence comprises (or consists of) a sequence that has at least about 80%, 85%, 90%, 95%, 99% or 100% sequence identity to SEQ ID NO:1, 2, 3, or 4.

Embodiment 5. The recombinant polypeptide according to any one of embodiments 1-4, wherein the DNA helicase is Rep helicase or a fragment thereof (e.g., a catalytically active fragment thereof).

Embodiment 6. The recombinant polypeptide according to any one of embodiments 1-5, wherein the DNA helicase amino acid sequence comprises (or consists of) a sequence that has at least about 80%, 85%, 90%, 95%, 99% or 100% sequence identity to SEQ ID NO:5 or 20.

Embodiment 7. The recombinant polypeptide according to any one of embodiments 1-6, wherein the T5 DNA polymerase amino acid sequence is operably linked to the DNA helicase amino acid sequence via a peptide linker.

Embodiment 8. The recombinant polypeptide of embodiment 7, wherein the peptide linker comprises (or consists of) an amino acid sequence having at least about 80%, 85%, 90%, 95%, 99% or 100% sequence identity to SEQ ID NO:6, 7, 8, or 9.

Embodiment 9. The recombinant polypeptide according to any one of embodiments 1-8, comprising (or consisting of) an amino acid sequence having at least about 80%, 85%, 90%, 95%, 99% or 100% sequence identity to SEQ ID NO:10, 11, 22, or 23.

Embodiment 10. The recombinant polypeptide according to any one of embodiments 1-9, which is capable of replicating or mutagenizing a nicked DNA.

Embodiment 11. The recombinant polypeptide according to any one of embodiments 1-10, wherein the DNA polymerase has a higher speed as compared to the DNA helicase.

Embodiment 12. A nucleic acid encoding the recombinant polypeptide of any one of embodiments 1-11.

Embodiment 13. An expression cassette comprising the nucleic acid of embodiment 12.

Embodiment 14. The expression cassette of embodiment 13, further comprising a T7 promoter.

Embodiment 15. The expression cassette of any one of embodiments 13-14, further comprising a 5′-untranslated region (5′ UTR) comprising (or consisting of) a sequence having at least about 80%, 85%, 90%, 95%, 99% or 100% sequence identity to SEQ ID NO:16.

Embodiment 16. A helper vector comprising the nucleic acid sequence of embodiment 12 or the expression cassette of any one of embodiments 13-15.

Embodiment 17. A DNA replisome complex, comprising:
1) a target double stranded DNA (dsDNA) comprising a DNA nickase initiation sequence;
2) a corresponding DNA nickase that is capable of nicking the target dsDNA at the initiation sequence; and
3) a recombinant polypeptide according to any one of embodiments 1-11, wherein the recombinant polypeptide is capable of replicating the nicked dsDNA.

Embodiment 18. The DNA replisome complex of embodiment 17, wherein the DNA nickase is CisA or comprises (or consists of) an amino acid sequence having at least about 80%, 85%, 90%, 95%, 99% or 100% sequence identity to SEQ ID NO:24.

Embodiment 19. The DNA replisome complex according to embodiment 17 or 18, wherein the DNA nickase initiation sequence comprises (or consists of) a sequence having at least about 80%, 85%, 90%, 95%, 99% or 100% sequence identity to SEQ ID NO:13.

Embodiment 20. The DNA replisome complex of embodiment 17, wherein the target dsDNA further comprises a termination sequence.

Embodiment 21. The DNA replisome complex of embodiment 20, wherein the termination sequence comprises (or consists of) a sequence having at least about 80%, 85%, 90%, 95%, 99% or 100% sequence identity to SEQ ID NO:14 or 21.

Embodiment 22. The DNA replisome complex according to any one of embodiments 17-21, wherein the target dsDNA further comprises a target DNA sequence operably linked downstream of the initiation sequence, wherein the target DNA sequence comprises one or more genes.

Embodiment 23. A nucleic acid sequence comprising a target double stranded DNA (dsDNA) as described in any one of embodiments 17-22.

Embodiment 24. An expression cassette comprising the nucleic acid sequence of embodiment 23.

Embodiment 25. A target vector comprising the nucleic acid sequence of embodiment 23, or the expression cassette of embodiment 24.

Embodiment 26. A host cell comprising the DNA replisome complex according to any one of embodiments 17-22; a DNA nickase or a vector encoding a DNA nickase; a helper vector of embodiment 16; and/or a target vector of embodiment 25.

Embodiment 27. A targeted DNA replication or mutagenesis system comprising:
1) a target dsDNA comprising a DNA nickase initiation sequence, or a target vector of embodiment 25;
2) a DNA nickase, or a host cell comprising the DNA nickase; and
3) a recombinant polypeptide of any one of embodiments 1-11 or the helper vector of embodiment 16;
wherein the DNA nickase is capable of nicking the dsDNA or the target vector at the initiation sequence and the recombinant polypeptide is capable of replicating or mutagenizing the nicked dsDNA or target vector.

Embodiment 28. A kit comprising:
1) a target vector comprising a DNA nickase initiation sequence;
2) a host cell comprising a DNA nickase (e.g., CisA);
3) a helper vector according to embodiment 16;
4) packaging materials; and
instructions for inserting a target DNA sequence into the target vector downstream of the DNA nickase initiation sequence; and contacting the host cell with the helper vector and the target vector comprising the target DNA sequence under conditions suitable for the vectors to enter the host cell and mutagenize the target vector.

Embodiment 29. A method comprising contacting a cell with the nucleic acid of embodiment 12, an expression cassette of any one of embodiments 13-15 or the helper vector of embodiment 16.

Embodiment 30. The method of embodiment 29, wherein the cell expresses a DNA nickase.

Embodiment 31. The method of embodiment 29, further comprising contacting the cell with a nucleic acid encoding a DNA nickase (e.g., a nucleic acid is present within a vector).

Embodiment 32. The method of any one of embodiments 29-31, further comprising contacting the cell with a target dsDNA (e.g., a target vector as described herein), wherein the target dsDNA comprises a corresponding DNA nickase initiation sequence operably linked upstream of a target DNA sequence, and optionally, a DNA nickase termination sequence operably linked downstream of the target DNA sequence.

Embodiment 33. A method of mutagenizing a target DNA sequence comprising introducing the target DNA sequence into a target dsDNA downstream of a DNA nickase initiation sequence, and assembling a DNA replisome complex as described in any one of embodiments 17-21.

Embodiment 34. The method of embodiment 33, wherein the target dsDNA comprising the target DNA sequence is comprised within a vector.

Embodiment 35. A method of mutagenizing a target DNA sequence in a cell comprising contacting the target DNA sequence with a recombinant polypeptide as described in any one of embodiments 1-11, wherein the target DNA sequence is operably linked downstream of a DNA nickase initiation sequence; and wherein the cell expresses a corresponding DNA nickase; under conditions suitable for the DNA nickase to nick the initiation sequence and for the recombinant polypeptide to mutagenize the target DNA sequence.

Embodiment 36. The method of embodiment 35, further comprising contacting the cell with the nucleic acid of embodiment 12, an expression cassette of any one of embodiments 13-15 or the helper vector of embodiment 16, under conditions suitable to express the recombinant polypeptide.

Embodiment 37. The method of embodiment 35 or 36, further comprising contacting the cell with a nucleic acid encoding a DNA nickase (e.g., a nucleic acid is present within a vector), under conditions suitable to express the DNA nickase.

Embodiment 38. The method of any one of embodiments 35-37, further comprising contacting the cell with a target vector comprising the target DNA sequence operably linked downstream of the DNA nickase initiation sequence.

Embodiment 39. A method of mutagenizing a target DNA sequence comprising contacting a host cell that expresses a DNA nickase with: 1) a target vector comprising a corresponding DNA nickase initiation sequence operably linked to the target DNA sequence; and 2) a helper vector as described in embodiment 16; under conditions suitable for the vectors to enter the host cell; for the DNA nickase to nick the initiation sequence; and for the recombinant polypeptide to mutagenize the target DNA sequence.

Embodiment 40. A T5 DNA polymerase comprising a I308V mutation, wherein the substitution and position are in reference to SEQ ID NO:1.

Embodiment 41. A recombinant polypeptide comprising a T5 DNA polymerase that comprises a I308V mutation, and wherein the substitution and the position are in reference to SEQ ID NO:1.

Embodiment 42. The T5 DNA polymerase or recombinant polypeptide according to any one of embodiments 40-41, where in the T5 DNA polymerase comprises (or consists of) an amino acid sequence having at least about 80%, 85%, 90%, 95%, 99% or 100% sequence identity to SEQ ID NO:3, or 4.

Embodiment 43. The method of embodiment 35, wherein the cell comprises chromosomal DNA comprising a nucleic acid sequence encoding the recombinant polypeptide.

### Certain Definitions

The term “nucleic acid” and “polynucleotide” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule.

“Recombinant nucleic acid molecule” is a combination of nucleic acid sequences that are joined together using recombinant nucleic acid technology and procedures used to join together nucleic acid sequences as described, for example, in Sambrook and Russell (2001). As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least about one exon and (optionally) an intron sequence.

A “vector” is defined to include, inter alia, any plasmid, cosmid, phage or binary vector in double or single stranded linear or circular form which may or may not be self-transmissible or mobilizable, and which can transform a host cell either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least about one of its components is heterologous with respect to at least about one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, development-specific promoters, regulatable promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Constitutive expression” refers to expression using a constitutive or regulated promoter. “Conditional” and “regulated expression” refer to expression controlled by a regulated promoter.

“Expression” refers to the transcription and/or translation of an endogenous gene, heterologous gene or nucleic acid segment, or a transgene in cells. Expression may also refer to the production of protein.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., dehydroalanine, homoserine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, a-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T.W. Greene, Protecting Groups In Organic Synthesis; Wiley: New York, 1981, and references cited therein) The term also comprises natural and unnatural amino acids bearing a cyclopropyl side chain or an ethyl side chain.

The terms “polypeptide” and “protein” are used interchangeably herein. A protein molecule may exist in a purified form or may exist in a non-native environment such as, for example, a transgenic host cell or bacteriophage. Fragments and variants of the disclosed proteins or partial-length proteins encoded thereby are also encompassed by the present invention. By “fragment” or “portion” is meant a full length or less than full length of the amino acid sequence of a protein.

By “portion” or “fragment,” as it relates to a nucleic acid molecule, sequence or segment of the invention, when it is linked to other sequences for expression, is meant a sequence having at least about 80 nucleotides, more preferably at least about 150 nucleotides, and still more preferably at least about 400 nucleotides. If not employed for expressing, a “portion” or “fragment” means at least about 9, preferably 12, more preferably 15, even more preferably at least about 20, consecutive nucleotides, e.g., probes and primers (oligonucleotides), corresponding to the nucleotide sequence of the nucleic acid molecules of the invention.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

The terms “introduce to a cell” and “introduction to a cell” refers to contacting a cell with a composition described herein for intracellular delivery or administration of the composition. The DNA replication or mutagenesis system/components of such a system can be provided as isolated or purified protein, RNA, a vector or any combination thereof. Thus, the methods of introduction can be a combination of delivery methods. For example, a polypeptide or an RNA can be introduced indirectly via intracellular delivery/expression of a vector comprising a nucleic acid encoding the recombinant polypeptide or the RNA. Non-limiting examples of vector delivery methods include transformation (e.g., transduction), viral and non-viral based delivery, nanoparticle delivery, liposomal delivery, etc. Alternatively, polypeptide(s) and RNA can be introduced through the use of non-limiting examples of nanoparticles, liposomes, electroporation, microinjection, and gene gun, etc.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “comparison window” makes reference to a contiguous and specified segment of an amino acid or polynucleotide sequence, wherein the sequence in the comparison window may comprise additions or deletions (i.e., gaps) compared to the reference sequence (which does not comprise additions or deletions) for optimal alignment of the two sequences. Generally, the comparison window is at least about 20 contiguous amino acid residues or nucleotides in length, and optionally can be 30, 40, 50, 100, or longer.

As used herein, “percentage of sequence identity” means the value determined by comparing two optimally aligned sequences over a comparison window, wherein the portion of the polypeptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least about 70%, 71%, 72%, 73%, 74%, 75%, 76%, 77%, 78%, or 79%, at least about 80%, 81%, 82%, 83%, 84%, 85%, 86%, 87%, 88%, or 89%, at least about 90%, 91%, 92%, 93%, or 94%, and at least about 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least about 70%, at least about 80%, 90%, or at least about 95%.

The term “substantial identity” in the context of a peptide indicates that a peptide comprises a sequence with at least about 70%, 71%, 72%, 73%, 74%, 75%, 76%, 77%, 78%, or 79%, 80%, 81%, 82%, 83%, 84%, 85%, 86%, 87%, 88%, or 89%, at least about 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity or complementarity for the test sequence(s) relative to the reference sequence, based on the designated program parameters.

The invention will now be illustrated by the following non-limiting Example.

### Example 1

### Evolutionary Innovation by Hypermutation Using a Targeted Artificial DNA Replisome

Extensive exploration of a protein's sequence space for improved or new molecular functions requires in vivo evolution with large populations. But disentangling the evolution of a target protein from the rest of the proteome is challenging. As described herein, a protein complex of Targeted Artificial DNA Replisome (TADR) was designed in live cells to processively replicate an arbitrary target gene with errors. It enhanced mutation rates of targets up to 2.3×10⁵-fold with only a 78-fold increase in off-target mutagenesis. It was used to evolve itself to increase error rate and increase the efficiency of an efflux pump while simultaneously expanding the substrate repertoire. TADR enables multiple simultaneous substitutions to discover functions inaccessible by accumulating single substitutions, affording great potential for solving hard problems in molecular evolution, development of biologic drugs, and industrial catalysts.

### INTRODUCTION

The evolutionary innovation of new protein functions is central to Darwinian adaptation. For example, bacterial efflux pumps evolved into antibiotic resistance proteins (Lupo et al., Frontiers in Microbiology, 3 (18): (2012)). Natural evolution depends in complex ways on population size, mutation rate, and the shape of the adaptive landscape. Because evolution in nature occurs over decades or longer, it is difficult to study and also difficult to apply to making new enzymes for industry and medicine. The tool to solve this problem would target enhanced mutagenesis to specific genes in vivo, such as a plasmid that carries gene(s) for the protein(s) of interest. The in-vivo approach creates larger populations with mutation combinations beyond that accessible by in-vitro directed evolution (Packer et al., Nature Reviews Genetics, 16 (7): 379-394, (2015)). One milliliter of overnight bacterial culture carries cells in billions—the theoretical size of mutant library, whereas directed evolution experiments, which rely on in vitro chemistry of error-prone polymerase chain reaction (PCR), use no more than several million variants by pooling colonies from many electroporations (Gaudelli et al., Nature 551, 464-471 (2017)). Targeting specific genes prevents deleterious mutations elsewhere from obscuring beneficial mutations in the target gene. Enhanced mutagenesis speeds up evolution from decades to hours.

The ideal in vivo mutagenesis tool would a) target any gene-sized (or larger) regions of DNA and show low off-target mutagenesis, b) have a high mutation rate (e.g., that can be turned on and off) and include all types of nucleotide substitutions and c) be easy to use and not limit the type of trait that can be evolved. No tools currently satisfy all three requirements.

As described herein, in this Example a three-protein complex of a viral nickase, bacterial Rep helicase, and an error-prone DNA polymerase was designed to copy one strand of a target plasmid with errors and avoids these limitations by acting as a Targeted Artificial DNA Replisome (TADR). An error-prone DNA polymerase introduces mutations to speed evolution, and nickase initiation and termination sequences define the target region for mutagenesis.

Thus, described herein is an example of an engineered artificial DNA replisome with an enhanced mutation rate within a specifically targeted region. This Targeted Artificial DNA Replisome (TADR) fulfills the three requirements of targeting, mutagenesis, and trait flexibility mentioned above. It minimizes the interference with natural cellular processes by its simplicity, but is complex enough to target entire genes or multiple genes with the full spectrum of mutations.

### Results

### Designing a Replisome in Live Cells to Accelerate Protein Evolution.

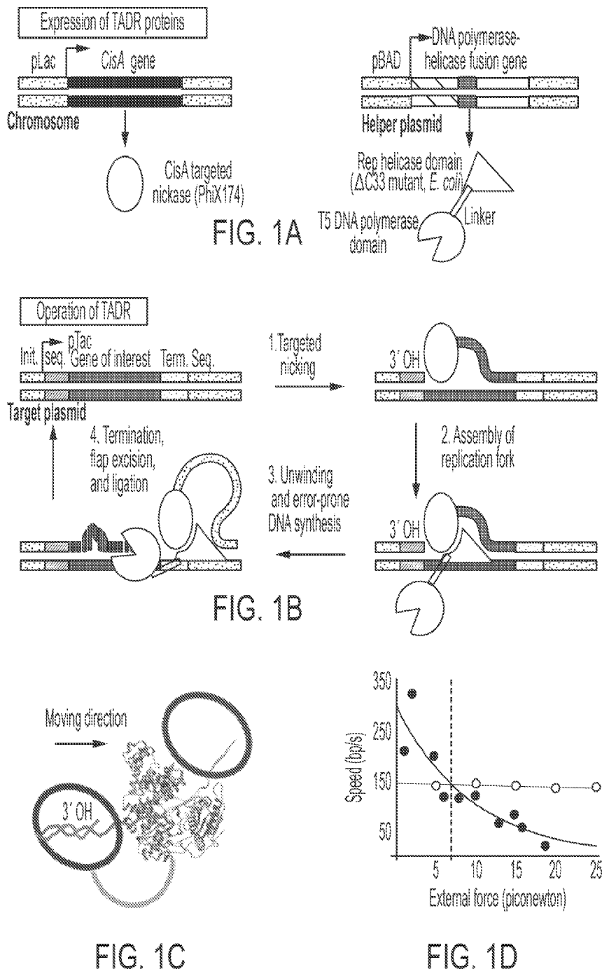

Most mutations originate from errors during copying of DNA by a multiprotein complex known as the DNA replisome. In the bacterium Escherichia coli, this protein complex consists of 18 components with a total mass of 1 MDa (Fernandez-Leiro et al., eLife, 4 e11134, (2015)). It was hypothesized that fusing an error-prone DNA polymerase to Rep helicase would create an error-prone DNA replisome (FIG. 1A-1B). The close proximity of the error-prone DNA polymerase would favor its use in the replisome complex resulting in a high mutation rate (FIG. 1C). This error-generating replisome required nontrivial coordination between the two molecular motors of helicase and DNA polymerase (as illustrated in FIG. 1D and FIG. 11). If DNA polymerase is slower than Rep helicase, the unwound single-strand DNA will build up between the two motors, promoting destruction by nucleases abundant in the cell (Lovett, EcoSal Plus, 4 (2): 10.1128/ecosalplus.1124.1124.1127, (2011)) and thus causing collapse of the replisome. If the DNA polymerase is faster than Rep helicase, a collision between them might dislodge the helicase. Single-molecule studies showed that both motors tolerated collisions: Rep helicase maintained a nearly constant speed when subjected to external forces (Arslan et al., Science (New York, N.Y.), 348 (6232): 344-347, (2015)), while T7 DNA polymerase slowed down (Maier et al., Proceedings of the National Academy of Sciences, 97 (22): 12002-12007, (2000))(FIG. 1D). Importantly, the speeds intersected predicting that applying an external force on T7 DNA polymerase could slow it down so that both motors act at the same speed. As Rep helicase unwinds the DNA, the faster T7 DNA polymerase bumps into it. This bumping creates an external force that does not affect Rep helicase, but slows down T7 DNA polymerase leading to coordinated action of both. More details of the biophysical plausibility of the coordination between unwinding of DNA by the Rep helicase and DNA copying by the polymerase is presented in the Methods of this example. The initial attempt of building TADR with T7 DNA polymerase failed since the active site was too close to the linker site at the C-terminus. Therefore, a close relative was investigated: bacteriophage T5 DNA polymerase, where the C-terminus is farther from the active site. In addition, T5 was chosen over the E. coli DNA polymerase I (Camps, et al., PNAS. 100, 9727-9732 (2003); and Halperin, et al., Nature 560, 248-252 (2018)). T5 is a fast polymerase like T7 (Andraos, et al., J. Biol. Chem. 279, 50609-50618 (2004)), while the E. coli DNA polymerase I is slower than Rep (Maier, et al., PNAS. 97, 12002-12007 (2000)), and thus not expected to form a stable replisome according to FIG. 1D. The active site of T5 polymerase lies farther from the C terminus than in T7 polymerase (Table 3), suggesting that the fusion protein would maintain polymerase activity.

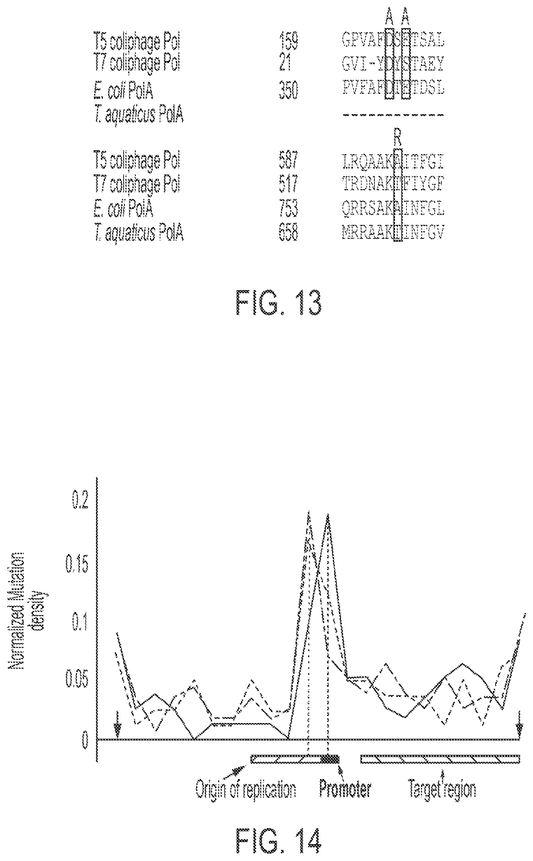

In this example, a flexible 81-amino acid linker connected the N-terminus of Rep helicase to the C-terminus of T5 DNA polymerase (FIG. 1A). This DNA polymerase carried three amino acid substitutions (D164A, E166A, A593R) to increase its error rate. Substitution of these residues increased the error rate in homologous polymerases (FIG. 13) (Morrison et al., Proc Natl Acad Sci USA, 88 (21): 9473-9477, (1991); Camps et al., Proc Natl Acad Sci USA, 100 (17): 9727-9732, (2003)).

It was further hypothesized that the enhanced mutagenesis could be restricted to a target region (e.g., the gene(s) of interest) by adding an initiation sequence for CisA before the target region and a termination sequence (Fluit et al., Virology, 154 (2): 357-368, (1986)) for CisA after the target region, although experiments in this Example indicate that enhanced mutagenesis may occur on the entire target plasmid and read-through may occur. Copying a template DNA strand (e.g., starting at the initiation sequence and stopping at the termination sequence) creates a new DNA strand annealed to the template strand, while the non-template strand is unbound as a flap (FIG. 1B). This flap is naturally excised (Lyamichev et al., Science, 260 (5109): 778-783, (1993)), and the resulting nick ligated (Uphoff et al., Proceedings of the National Academy of Sciences, 110 (20): 8063-8068, (2013)). If an error-prone polymerase carries out the copying, then the copied DNA likely contains a mutation. This copying process repeats to introduce additional mutations. As will be detailed later, high selectivity for the target plasmid over the chromosome was achieved, but the selectivity within the target plasmid was lower than the high selectivity for target plasmid over chromosome.

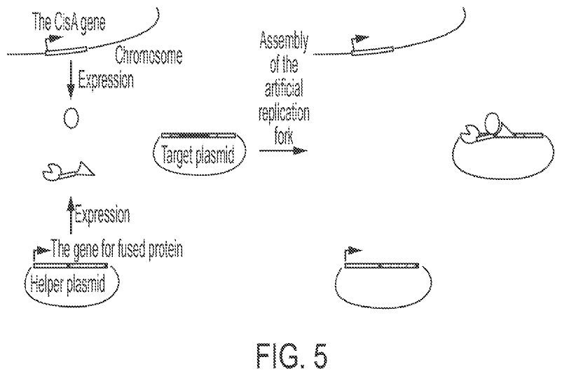

Three DNA elements were added to E. coli to create a targeted error-prone artificial replisome: a constitutively-expressed, chromosomal CisA gene, a target vector plasmid and a helper vector plasmid (FIG. 1A-1B and FIG. 5). The target vector contained the gene(s) of interest targeted for mutagenesis between the CisA initiation and termination sequences. To quantify the increase in mutagenesis, mutagenesis was targeted to the gene for a kanamycin resistance protein (aminoglycoside phosphotransferase (3)-IIIa). This gene, named kanR*, carried a single nucleotide substitution (A785C), which encodes an E262A amino acid substitution that inactivates the kanamycin resistance (Thompson et al., J Biol Chem, 274 (43): 30697-30706, (1999)). Mutations that reverse this substitution will restore gene function, allowing the cells to grow in the presence of kanamycin. Resistant colony-forming unit (CFU) per pl of full density culture (frequency of resistant CFU per cell plated) is used as a readout for mutagenic capacity. The helper plasmid contains a gene encoding the fusion of the error-prone T5 DNA polymerase to the Rep helicase under the control of a T7 promoter. (The host cell also contains a chromosomal gene to express the T7 RNA polymerase and a deletion of the chromosomal Rep gene to eliminate potential competition with the fusion protein.)

### Individual Components of TADR are Functional In Vivo.

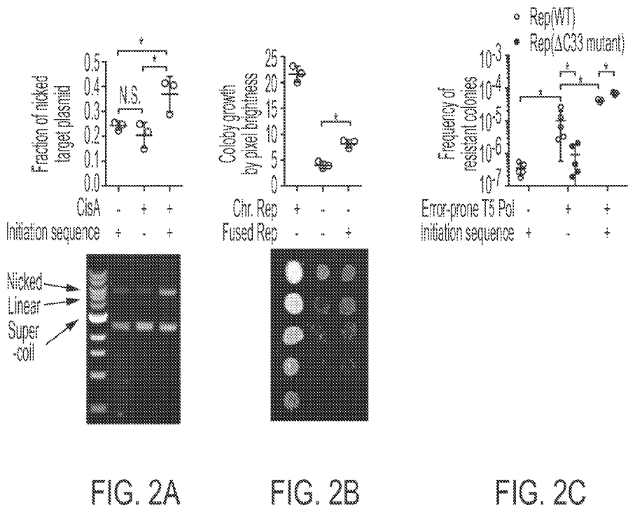

The in vivo activity of CisA was measured by its ability to nick DNA at the CisA initiation site. Bacteria containing constitutively-expressed, chromosomal CisA gene and target plasmid carrying kanR* (but no helper plasmid) were propagated for multiple generations. The target plasmid was extracted and separated by gel electrophoresis into the supercoiled, nicked and linear forms, FIG. 2A. Substantial nicking of the target plasmid occurred when both CisA and the CisA initiation sequence were present, consistent with CisA protein being active. When either CisA protein or its initiation sequence was absent, a lot less nicking occurred. The small amount of untargeted nicking observed is likely non-specific hydrolysis during the alkaline lysis step of the plasmid extraction (Clemson et al., Biotechnology and Applied Biochemistry, 37 (3): 235-244, (2003)).

The Rep helicase activity of the T5 DNA polymerase-Rep helicase fusion was measured by its ability to alleviate growth retardation caused by nalidixic acid in a strain lacking endogenous Rep helicase (Henderson et al., PLOS ONE, 10 (5): e0128092, (2015)). Bacteria normally tolerate low levels of nalidixic acid, but bacteria lacking endogenous Rep helicase grow more slowly because nalidixic acid inhibits DNA gyrase and topoisomerase. Introducing a helper plasmid, which adds expression of the T5 DNA polymerase-Rep helicase fusion protein, into bacteria lacking endogenous Rep helicase partially alleviated the nalidixic acid inhibition of growth (FIG. 2B and FIG. 8), which confirmed that the fusion protein maintained Rep helicase activity.

The DNA polymerase activity of the T5 DNA polymerase-Rep helicase fusion protein was measured by its capacity to mutate the target, kanR* and restore kanamycin resistance. The bacteria, in this Example, contained all three elements of the targeted error-prone artificial replisome: a constitutively-expressed, chromosomal CisA gene, the target plasmid carrying the kanR* gene flanked by the initiation and termination sequences, and the helper plasmid expressing the T5 DNA polymerase-Rep helicase fusion protein. These cells were picked from a transformation plate, propagated in liquid for ten generations to accumulate mutations and then plated onto kanamycin-containing media for selection. FIG. 2C shows that the treatment (third column, light gray dots) increased 128-fold than the control with wildtype T5 DNA polymerase (first column, light gray dots) in the frequency of kanamycin-resistant colonies; and, 4.2-fold than the control whose target plasmid was without initiation sequence (second column, light gray dots). These results demonstrated qualitatively that the fusion protein provided error-prone DNA polymerase activity which was able to distinguish non-target from target as marked by the initiation sequence.

### Self-Evolution of TADR for Increase in Mutation Rate.

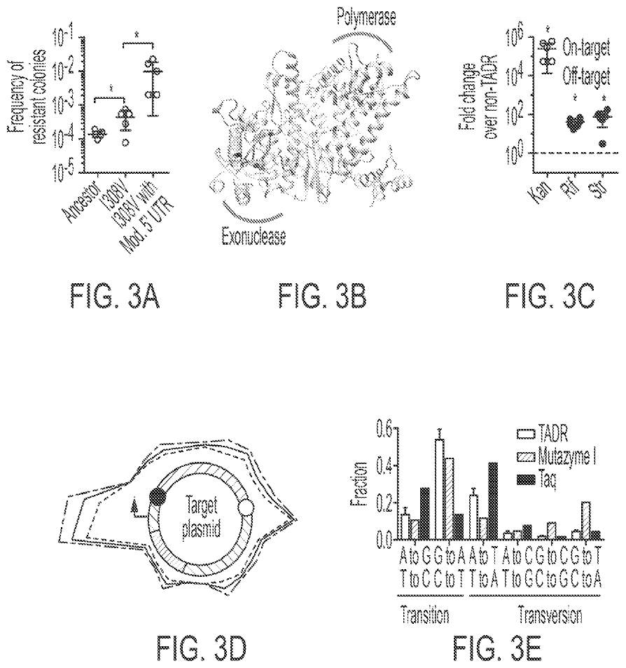

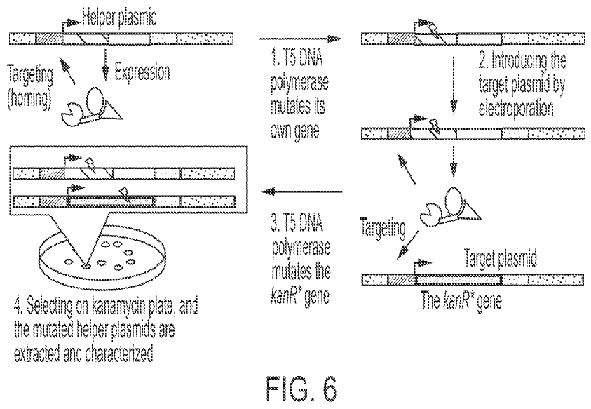

To optimize performance of TADR and as a proof-of-concept experiment, self-evolution of the error-prone T5 DNA polymerase increased the mutation rate 3.2-fold (FIG. 3A, ANOVA, α=0.05, P=0.033, n=5). To target mutations to the T5 DNA polymerase, the 30-bp CisA initiation and termination sequences were inserted on helper plasmid to flank the polymerase portion of the T5 DNA polymerase-Rep helicase fusion gene. The termination sequence was in-frame to maintain protein expression of the Rep helicase portion of the fusion protein. TADR cells with only this helper plasmid but no target plasmid were cultured for the T5 DNA polymerase to mutate its own gene. Target plasmid with kanR* was then introduced by transformation for selection. The target plasmid contained the kanR* gene also flanked by the 30-bp CisA initiation and termination sequences. To select for increased mutation rate, the incubation time was reduced before selection for kanamycin resistance (Methods of this example, FIG. 6). This approach identified a variant DNA polymerase, which, in separate experiments, increased the number of kanamycin-resistant CFU 3.2 fold (FIG. 3A). This polymerase variant carried a single I308V substitution (FIG. 3B). This substitution is unlikely to change the accuracy of the polymerase or increase the error rate of the polymerase, but may stabilize it or increase its expression, thereby increasing the error frequency. This substitution lies in the exonuclease (proofreading) domain, which is already inactivated by two substitutions (D164A and E166A; FIG. 3A) (Morrison et al., Proc Natl Acad Sci USA, 88 (21): 9473-9477, (1991)), and also lies outside the catalytic pocket (8.8 A from D164), so it is unlikely to affect catalytic activity. The change in shape of the hydrophobic side chain from Ile to Val may alter the protein stability or increase the expression of the protein, thereby increasing the observed mutation rate.

### Maximizing On-Target and Minimizing Off-Target Mutagenesis.

Engineering a 5′ untranslated region (5′ UTR) (by designing a stem-loop into the 5′ UTR) upstream the gene encoding the fusion protein increased the mutation rate up to 150-fold (FIG. 3A), likely by increasing the amount of fusion protein, whose expression level was calculated to increase 37-fold (Table 1).

Since TADR uses a Rep helicase from E. coli, it may cause off target mutations in the chromosome where Rep helicase normally participates in DNA replication (Syeda, A. H. et al. Nucleic Acids Res. 47, 6287-6298, (2019)). Consistent with this expectation, removal of the initiation sequence for CisA decreased mutagenesis only 4.2-fold, FIG. 2C. To improve the targeting, the 33 C-terminal amino acids of Rep helicase were deleted in the fusion protein. These amino acids bind Rep helicase to the innate replisome (Guy, C. P. et al. Mol. Cell 36, 654-666 (2009)). The fusion containing RepΔC33 showed a 16-fold increase in selectivity. Removal of the initiation sequence now decreased mutagenesis 67-fold (FIG. 2C, dark gray dots, third over second column).

To further increase selectivity, it was reasoned that the error-prone T5 DNA polymerase in the fusion protein, even with RepΔC33, could bump into innate replisomes by random diffusion and thus mutating the chromosome. So far, the experiments were all conducted in rich media (e.g., LB broth), where a single cell contains multiple copies of the chromosome, which replicated continuously throughout the cell cycle (Hiraga, S., et al., Mol. Cell 1, 381-387, (1998)). These innate replisomes provide sites where the error-prone T5 DNA polymerase could mutate the chromosome. It was hypothesized that reducing the number of innate replisomes would suppress off-target mutagenesis. One approach is growing cells in minimal medium, where each cell has only one copy of the chromosome and where chromosomal replication occurs in only a fraction of the cell cycle (Reyes-Lamothe, R. et al., Nat. Rev. Microbiol. 17, 467-478, (2019)). The lower numbers of innate replisomes would minimize their exposure to the error-prone T5 DNA polymerase.

Cells of the previous control to measure non-targeted mutagenesis (FIG. 2C, second column), whose target plasmid was without initiation sequence, failed to grow in minimal media, while those of the treatment, with initiation sequence, grew normally. A similar pattern was seen in LB and discussed in detail in later the section, “Toxicity of the target plasmid without initiation sequence”. As a consequence, a new control was needed to measure off-target mutagenesis.

Spontaneous resistance to rifampicin and to streptomycin are established traits to allow measurement of mutation rates on a chromosome (Halperin et al., Nature 560, 248-252 (2018); Lee et al., Proc. Natl. Acad. Sci. USA 109, E2774-E2783 (2012)). Since chromosomal and plasmid-borne traits differ in type and in the number of copies, the mutation rate of a mutagenic system as measured by resistance to an antibiotic was normalized to that of its non-mutagenic counterpart as done previously by Camps et al. (Proc. Natl. Acad. Sci. USA 100, 9727-9732 (2003)). The resulting fold-change adjusts for the different genetic underpinnings of resistance to different markers and thus provides a proxy for a universal mutation rate.

FIG. 3C shows that the engineered and evolved TADR, when induced in minimal media for mutagenesis, had high activity and selectivity for mutations on target plasmid. The on-target mutagenesis of TADR increased 2.37×10⁵-fold compared to that of non-TADR baseline. The off-target mutagenesis increased slightly: 39.7- and 77.5-fold for rifampicin- and streptomycin-resistance, respectively, corresponding to selectivities of 5970 and 3060-fold (obtained by dividing 2.37×10⁵ by 39.7 and 77.5). The agreement between the increases in off-target mutagenesis measured by different antibiotics (ANOVA, n=6, P=0.16) confirms that the normalization method allows comparison of the mutation rate of different traits on a plasmid versus on a chromosome.

To test if the mutagenic activity could be suppressed, glucose was added to minimal medium in which TADR cells were grown-glucose suppresses transcription from the promoters for cisA and the fusion protein by catabolite repression. The mutagenic activity was lowered by 112-fold with the presence of glucose than without (FIG. 16).

### Characterization of TADR by NGS Confirms the Designed Molecular Mechanisms for High Performance.

Next generation sequencing (NGS) revealed increased mutagenesis throughout the target plasmid with two hotspot regions (FIG. 3D shows mutational density defined as the number of mutations normalized to the total mutations of the sample per 200-bp window across the entire plasmid; a total of 264 point mutations were called in the three samples; see details in Methods and FIG. 14, FIG. 17). Both hotspots coincide with sites of transcription initiation or termination and can be explained by molecular collision-induced hypermutagenesis.

The two hotspots together only account for 11.5% of the target plasmid. Regarding the remaining 88.5%, the region (the gene(s) of interest) defined by the flanking initiation and termination sequences had 1.75-fold higher mutations per base pair (ANOVA, n=3, P=0.01) than lowest region (the plasmid backbone). Hence, TADR replisome reads through the termination sequence 57% of the time. Within the target region from initiation to termination sequences, mutational density did not decline as tested by linear regression across this region of 1596 bp (bin size, 100-bp; P=0.77). This observation confirmed the high processivity of the artificial replisome in vivo and implied the capacity of TADR to target much larger DNA fragments. Both the targeting and this high processivity although with read-throughs and a selectivity of 1.75—were expected from the design in FIG. 1. The high mutagenesis throughout the plasmid may be due to incomplete termination of transcription allowing the replisome to continue and mutate the rest of the plasmid. Another plasmid region with enhanced mutagenesis was the promoter region and part of the origin of plasmid replication, indicating that transcription and plasmid propagation interfered with the artificial replisome. The high mutation rate throughout the plasmid demonstrates the ability to target multiple genes for mutagenesis simultaneously.

The mutational spectrum of TADR is similar to those of Taq DNA polymerase and commercial error-prone DNA polymerase widely used for directed evolution (mutazyme I, Agilent Technologies) with a bias for transitions of G to A and C to T (FIG. 3E). The ratio of transitions over transversions did not differ between the mutational hot spot encompassing the promoter and part of the origin of plasmid replication and the rest of the plasmid (ANOVA, n=3, P=0.35). Overall, next generation sequencing confirmed that the designed artificial replisome copied one strand of target plasmid with error, acted processively and increased mutagenesis of the target plasmid, yielding a mutation spectrum similar to model error-prone DNA polymerases.

TADR Evolves New Molecular Functions with Large Mutational Step-Size in Exploring the Sequence Space.

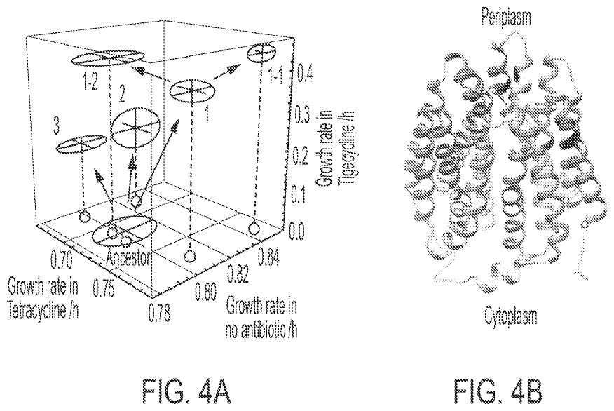

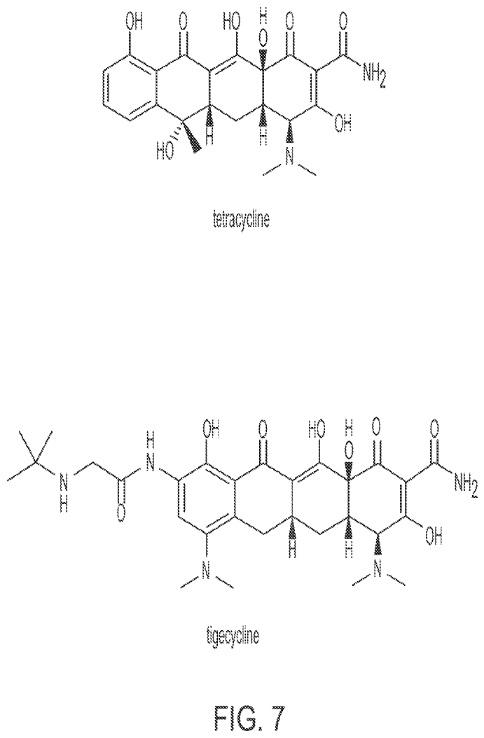

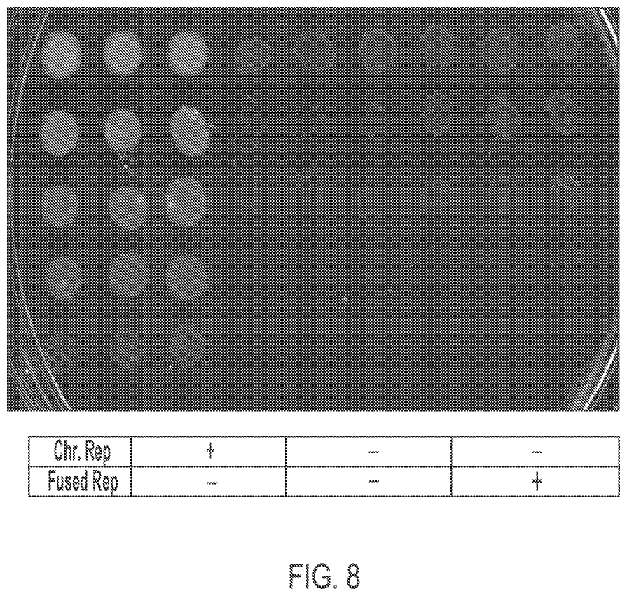

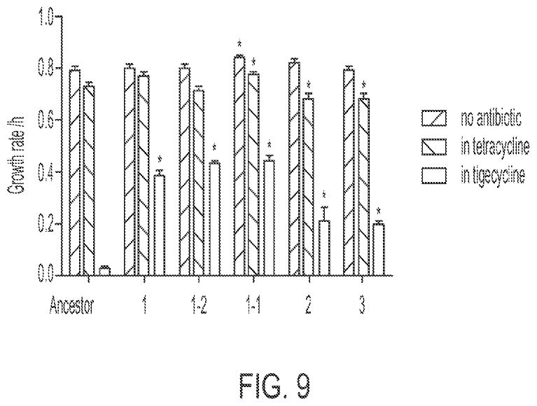

A tetracycline efflux pump (encoded by tetA(C)) was also evolved to confer resistance to an analog, tigecycline. This tetracycline derivative, used as a last resort antibiotic, contains a large side group that hinders the evolution of resistance (FIG. 7) (Linkevicius et al., Antimicrob Agents Chemother, 60 (2): 789-796, (2015)). TADR cells carrying both the helper plasmid and target plasmid containing the tetA(C) gene were grown in liquid culture to accumulate mutations and then selected by plating on tigecycline-supplemented medium. After two rounds of mutagenesis-selection, the growth rate in 8 ng/pl tigecycline increased up to 16-fold compared to the ancestor (wild-type pump) (ANOVA, α=0.05, n=5, P<0.005). All five mutants with increased resistance to tigecycline maintained or increased their resistance to tetracycline (FIG. 4A and FIG. 9). For example, Mutant 1-1 increased resistance to both tetracycline and tigecycline as compared to the ancestor. This ability to resist both antibiotics indicates that expanding the function of the tetracycline efflux pump did not require a tradeoff between resistance to tetracycline and to tigecycline.

Amino acid substitutions in the efflux pump occurred at the opening of the channel that directly contacted substrates as well as in peripheral locations that did not (FIG. 4B, black residues; mutant genotypes in Table 2). The first round of selection yielded double-substitution variants from all three parallel populations. Mutants 1 to 3 all contained the 1235F substitution at the opening of the channel, as well as another substitution, which differed between the three. Previous experiments showed that 1235F confers tigecycline resistance (Linkevicius et al., Antimicrob Agents Chemother, 60 (2): 789-796, (2015)), but Mutant 1 had resistance more than 1.8 fold those of Mutants 2, 3. Hence, the second substitution of S312F in Mutant 1 conferred further resistance to tigecycline in addition to I235F. Hence, TADR was able to identify beneficial mutations of more than a single substitution in one round. This large step size of exploring the sequence space enables overcoming fitness valleys, where the individual substitutions are not beneficial but their combination is (Weinreich, et al., Science 312, 111-114 (2006)). The best mutant (FIG. 4A, Mutant 1-1) not only evolved the highest level of resistance to tigecycline and increased resistance to tetracycline but also unexpectedly grew 6.2% faster in the absence of any antibiotic (ANOVA, n=5, α=0.05, P=0.023). These results suggested that the wildtype efflux pump was far from optimality. TADR achieved evolutionary innovation by expanding the substrate repertoire while reducing cellular toxicity of an efflux pump.

To specifically test for the ability to simultaneously introduce two substitutions, a double reversion assay was designed where the target region contained the gene for chloramphenicol resistance with two stop codons in the middle of the open reading frame. Emergence of chloramphenicol resistance requires simultaneous mutation of both stop codons to compatible amino acids. This double substitution requires finding one in hundreds of thousand possible double substitution variants. Eight chloramphenicol-resistant colonies emerged from plating 50 pl culture (8×10⁷ cells), and Sanger sequencing confirmed that three of them contained two mutated stop codons. Namely, chloramphenicol-resistant colonies emerged from a single cycle of mutagenesis selection, and the two synthetic stop codons were both mutated to amino acid-encoding codons (FIG. 12). The other five colonies may be false negatives in Sanger sequencing or false positives in selection. Since the cell contains ˜15 plasmid copies, plasmids containing repaired chloramphenicol acetylase genes are invariably diluted by unrepaired genes. Sanger sequencing may miss the repaired gene sequence within an excess of unrepaired gene. False positives in selection could result from off-target mutagenesis leading to chloramphenicol resistance.

In conclusion, TADR identified beneficial mutations of more than a single substitution in one round. This large step-size of exploring the sequence space makes overcoming fitness valleys possible where the individual substitutions are not beneficial but their combination is (Weinreich et al., Science, 312 (5770): 111-114, (2006)).

### Discussion

These results confirmed the successful design of a targeted artificial DNA replisome along with the activities of its individual components, and then demonstrated high performance of TADR. TADR directed high mutagenesis to target plasmid to find, for example, specific single or double mutations of gene(s) of interest in a large target window, while avoiding off-target mutagenesis such as on the chromosome in this Example.

The targeted artificial DNA replisome is complex enough to meet varied functional requirements of targeted mutagenesis. TADR replicated DNA processively as designed based on biophysical principles, thus the large target window, and was directed by the specific interaction between CisA and a 30-bp initiation sequence, thus the selectivity. Additional improvements by adding genetic (using the RepΔC33 mutant) and physiological (inducing mutations in minimal media) changes further enhanced selectivity between target plasmid and non-target chromosome.

TADR is simple enough to be modular. One feature of this modularity is that TADR can be almost shut off (lowered by 112-fold) while the cell and the target plasmid continue propagating (FIG. 16). The advantage is simpler isolation of the beneficial genetic construct. After a signal is detected in screening, the mutant virus or plasmid needs to be amplified in the host cell and purified to obtain the genetic construct. Amplification requires dozens of generations. If additional mutations occur during this amplification, they can confound the result. Previous tools that rely on virus-cell interaction or plasmid propagation cannot be turned off. In an E. coli polA DNA polymerase-based mutagenesis system (Camps, et al., PNAS 100, 9727-9732 (2003)), which is not a modular system, the polA DNA polymerase synthesizes ColE1 plasmid with error, and the enzyme also participates in chromosomal replication. As a result, the selectivity of the E. coli polA DNA polymerase-based system is 15.3-fold lower than that of TADR (390 versus 5970; the off-target mutation rate used to calculate selectivity was measured with rifampicin resistance in both systems).

Other potential benefits of simplicity and modularity include the ability to move TADR into cell-free systems. CisA-mediated phage replication was reconstituted in cell-free systems (Eisenberg, S., et al., Proc. Natl. Acad. Sci. USA 74, 3198-3202, (1977)), suggesting that TADR may work outside cells as well. In certain circumstances, the ability to identify, in a single round, beneficial double mutants makes TADR superior to error-prone PCR (which rarely report these mutants) in producing genetic diversity.

Compared to the high selectivity between target plasmid and non-target chromosome mutagenesis in this Example (FIG. 3C), TADR shows relatively lower selectivity within the plasmid (FIG. 3D). The plasmid does not contain other genes that could contribute to adaptation, so these mutations have little effect. One potential minor effect is slowing down the evolution of the target gene since deleterious mutations outside the target gene may obscure beneficial mutations within the target gene. The region outside the target region is the same order of magnitude in mutational density as the target gene, this slow down should not prevent evolution. On the positive side, this relatively lower selectivity suggests that mutation of multiple genes forming a biosynthetic pathway will likely be possible. One reason for the relatively lower selectivity might be low terminator efficiency (43%). In certain embodiments, adding additional copies of the terminator sequence may improve its efficiency. The fusion protein and CisA are currently expressed from a helper plasmid and chromosome, respectively. Expressing them from the same locus in a chromosome may increase genetic stability of TADR (by eliminating the helper plasmid). Alternatively, expressing them in the same plasmid will provide convenience for installment in other bacterial strains.

TADR demonstrates the power of both evolutionary innovation and rational design. Harnessing evolutionary innovation for technology is inspired by the innumerable adaptations seen in natural systems. The potential has mostly been unrealized because biological systems involve multiple functional dependencies that are difficult to optimize. TADR overcomes these problems because its rational design leverages existing biological knowledge and excludes the complex dependencies of natural systems. The optimization of the system and its application harnesses the power of evolutionary innovation. TADR's artificial replisome is far simpler than the cellular equivalent, and therefore can be optimized by natural selection. TADR's targeting is stringent and simple, virtually eliminating off-target mutagenesis and thereby simplifying selection experiments. TADR can increase the breadth of antibiotic resistance while simultaneously reducing the cost. Looking forward, TADR is a powerful tool for evolution useful proteins and an example for further design of biological tools.

### Methods

### Strains and Materials

TADR was implemented in wild type E. coli K12 strain MG1655 from the Coli Genetic Stock Center at Yale. The MM294 derivative of this strain from the same center was used for the construction and propagation of plasmids. The CisA gene was PCR-amplified from PhiX174 RF1 DNA (ThermoFisher Scientific). Bacteria were grown in liquid LB broth (10 g NaCl₂, 10 g tryptone 5 g yeast extract per liter; Fisher BioReagents) or its agar plates (15 g agar per liter; Fisher BioReagents). Cells were recovered from electroporation in SOC medium (20 g tryptone, 5 g yeast extract, 4.8 g MgSO₄, 3.603 g dextrose, 0.5 g NaCl, 0.186 g KCl per liter; MP Biomedicals). Self-evolution of TADR experiment were carried out in minimal media (1 g (NH₄)₂SO₄, 7 g K₂HPO₄, 2 g KH₂PO₄, 0.1 g MgSO₄, 0.5 g sodium citrate, 4 g glycerol, 2 g yeast synthetic drop-out medium supplements (Sigma) per liter). Antibiotics were added accordingly: 30 ng/μl kanamycin (Fisher Scientific), 50 ng/μl ampicillin (IBI Scientific), 15 ng/μl chloramphenicol (Sigma), 2.5 ng/μl nalidixic acid (Fisher Biotech), 8 ng/μl tetracycline (Fisher Scientific) and 4 or 8 ng/μl tigecycline (Neta Scientific). Phire Hot Start II DNA Polymerase (ThermoFisher Scientific) was used for PCR reactions; QIAprep Spin Miniprep Kit (Qiagen), purifying plasmid; GeneJET Gel Extraction Kit (ThermoFisher Scientific), purifying PCR products; FastDigest BsaI restriction enzyme (ThermoFisher Scientific), digesting DNA; Rapid DNA Ligation Kit (both from ThermoFisher Scientific), ligation; and GeneJET Gel Extraction and DNA Cleanup Micro Kit, purifying ligation products for electroporation.

### Biophysical Rationale of Building an Artificial Replisome

The artificial replisome must simultaneously unwind DNA and copy the exposed strand to prevent degradation of the exposed ssDNA, which causes genetic instability. In addition, DNA synthesis must avoid premature termination within the target region. Connecting Rep helicase and T5 DNA polymerase with a flexible linker increases the local effective concentrations to an estimated 0.2-0.6 mM(Li et al., Protein Sci, 27 (9): 1600-1610, (2018)) in order to achieve both of these goals. The CisA protein recruits Rep helicase to the target region and stabilizes it. Rep helicase unwinds DNA at about 144 base-pairs/s (Arslan et al., Science (New York, N.Y.), 348 (6232): 344-347, (2015)), so DNA synthesis must initiate at least that fast to proceed simultaneously. The bimolecular association rate constant of T7 DNA polymerase (kₒₙ) for primed DNA substrate is 3.6×10⁷M⁻¹s⁻¹ (Luo et al., Proceedings of the National Academy of Sciences, 104 (31): 12610-12615, (2007)) and it was assumed T5 DNA polymerase associates at a similar rate. If the local effective concentration of polymerase is 0.2-0.6 mM, then the rate of association is 7200-22,000 s⁻¹, which far exceeds the rate of unwinding. Subsequent DNA synthesis by T5 DNA polymerase(Andraos et al., Journal of Biological Chemistry, 279 (48): 50609-50618, (2004)) starts without a pause (Christian et al., Proceedings of the National Academy of Sciences, 106 (50): 21109-21114, (2009)) and is faster than unwinding by Rep helicase (Arslan et al., Science (New York, N.Y.), 348 (6232): 344-347, (2015)). The faster polymerase would bump into the slower helicase, but single-molecule studies show that both proteins tolerate mechanical forces without being displaced (FIG. 1C) (Maier et al., Proceedings of the National Academy of Sciences, 97 (22): 12002-12007, (2000); Arslan et al., Science (New York, N.Y.), 348 (6232): 344-347, (2015)). The previous linking of similar polymerase to a slower helicase allowed them to proceed simultaneously along the DNA (Bedinger et al., Cell, 34 (1): 115-123, (1983); Kim et al., Cell, 84 (4): 643-650, (1996)). To ensure that the linker does not hinder T5 DNA polymerase, an intrinsically disordered peptide linker was used: human a-synuclein, which allows diffusion of two proteins along the linker at a rate comparable to the diffusion of free proteins (Grupi et al., Journal of Molecular Biology, 405 (5): 1267-1283, (2011)).

The increased effective concentration of the polymerase also promotes its rebinding to the DNA when it occasionally dissociates. An enhanced mutagenesis construct that relied on free polymerase copied only short stretches of DNA (Halperin et al., Nature, 560 (7717): 248-252, (2018)). The linking of polymerase to helicase promotes rebinding and resumption of DNA synthesis, thereby maintaining high processivity and permitting a wide target window for enhanced mutagenesis. These properties were confirmed by NGS characterization of target plasmids mutagenized by TADR (FIG. 3D).

### Construction of TADR

The host cells for TADR were a modified strain of E. coli strain MG1655 where the chromosomal araB gene was replaced with the gene for T7 RNA polymerase and the chromosomal lacZ gene replaced with the gene for CisA. The gene for T7 RNA polymerase was 10 PCR-amplified from E. coli strain BL21(DE3) (Coli Genetic Stock Center at Yale). One kilobase of DNA was PCR-amplified from each of upstream and downstream araB gene in the chromosome of E. coli strain MG1655. These three PCR products were integrated into a single fragment using the technique of fusion PCR(Szewczyk et al., Nature Protocols, 1 (6): 3111-3120, (2006)). This fragment served as a template to replace the chromosomal araB gene of the strain MG1655 with the gene for T7 RNA transcriptase using the technique of lambda Red recombineering(Datsenko et al., Proceedings of the National Academy of Sciences, 97 (12): 6640-6645, (2000)). In this strain, the gene for T7 RNA transcriptase was under the native pBAD promoter, and the gene for the fusion protein carried on plasmid could be expressed using T7 promoter with high expression level and stringent switch-off (by adding 0.2% glucose to media). In the same manner, the lacZ gene of this strain was replaced with the gene for CisA. The resulting strain provided host cells for TADR.

The helper plasmid expresses the linked Rep helicase and T5 DNA polymerase under the control of a T7 promoter. The gene for T5 DNA polymerase was PCR-amplified from T5 phage lysate, the gene for Rep helicase was PCR-amplified from the genomic DNA of strain MG1655, and a DNA fragment encoding a linker—a 20-amino acid glycine-rich peptide concatenated to an intrinsically disordered peptide from human a-synuclein (61 amino acids, codon-optimized for E. coli)—was chemically synthesized using the gBlock service of Integrated DNA Technologies. These three fragments were fused using fusion PCR into a gene that encodes a single polypeptide with T5 DNA polymerase and Rep helicase fused through the linker. This fragment was then inserted into a plasmid derived from pKD46 (Coli Genetic Stock Center at Yale, #7669) with an ampicillin resistance marker in the backbone using standard technique of molecular cloning(Green, (2012)). The original pKD46 did not grow at 37° C., and a mutant was selected from growth at this temperature and used in this study. The fused gene was expressed under T7 promoter. Several versions of this plasmid were made: one carried a gene for the wildtype T5 DNA polymerase and the other carried an error-prone version with three point-mutations as detailed in FIG. 13. The helper plasmids with RepΔC33 and the 5′ UTR-modification were made using the same method.

To construct the target plasmid, fusion PCR was used to make an operon where the constitutive Tac promoter expressed the kanR* gene together with the gene for green fluorescent protein. The two genes were flanked by an upstream initiation sequence and a downstream termination sequence. The operon was inserted into plasmid pACYC184 (Addgene, #37033) with a chloramphenicol resistance marker in the backbone. Another version of this plasmid was also made that did not carry the initiation sequence.

To construct the target plasmid for evolution in tigecycline, the tetA(C) gene encoding the tetracycline efflux pump was PCR-amplified from plasmid pBR322 (New England Biolabs), and inserted into the target plasmid just described, replacing the gene for green fluorescent protein.

The full system of TADR is schematically shown in FIG. 5.

### Nicking of Target Locus by CisA

Three transformations were carried out as shown in FIG. 2A: the target plasmid into cells without the chromosomal gene for CisA, the target plasmid without the initiation and termination sequences into cells with the chromosomal gene for CisA, and the target plasmid into cells with the chromosomal gene for CisA. Transformant colonies were selected on LB agar plates supplemented with 15 ng/μl chloramphenicol and inoculated to liquid LB cultures with chloramphenicol. After overnight growth in 37° C. shaken at 225 rounds per minute (the same growth conditions from here on unless specified otherwise), one ml culture was purified and eluted to 40 μl elution buffer, and 5 μl was used for electrophoresis (0.8% agarose gel, 110 v, 40 min; BioRad Mini-Sub Cell GT Systems). An image was taken using UVP Biodoc-it Imaging System and analyzed using ImageJ to calculate fraction of the nicked target plasmid with the equation: brightness of the band for nicked DNA/(brightness of the band for nicked DNA+brightness of the band for supercoiled DNA+brightness of the band for linear DNA).

### Helicase Activity of Rep-T5 DNA Polymerase Fusion Protein

Assay of sensitivity to nalidixic acid was used(Henderson et al., PLOS ONE, 10 (5): e0128092, (2015)). Three cell lines were prepared, namely TADR cells modified to have intact chromosomal rep, TADR cells (rep-) and TADR cells (rep-) transformed with helper plasmid (expressing the fusion protein). A Rep gene knockout mutation was P1-transduced from Keio-collection(Baba et al., Mol Syst Biol, 2 2006.0008-2006.0008, (2006)) into the host cell for using standard technique of molecular cloning(Green, (2012)). The helper plasmid was transformed into this rep-cell and selected on LB agar plate supplemented with 50 ng/μl ampicillin. From this cell line together with other two—the host cell for, its rep-derivative, single colonies were inoculated into liquid LB culture (ampicillin was added to cultures of the first cell line). After overnight growth, each culture was serially diluted with a dilution factor of ten, and 5 μl from each dilution was spotted on LB agar plate supplemented with 2.5 ng/μl nalidixic acid. After 16 h incubation in 37° C., a picture of the plate was taken using ImageRunner Advance C5250 Copier (Canon), and the background was subtracted using ImageJ for enhanced visual effect.

Increased Reversion Rate of Inactivated Kanamycin Resistance Gene (kanR*)

KanR* reversion assay was used. Three transformations were carried out, all into the host cell for TADR, as shown in FIG. 2C: the helper plasmid with the wildtype T5 polymerase co-transformed with the target plasmid; the helper plasmid with the error-prone T5 polymerase mutant, with the target plasmid without the recognition and termination sequences; the helper plasmid with the error-prone T5 polymerase mutant, with the target plasmid. Transformant colonies were selected on LB agar plates supplemented with 15 ng/μl chloramphenicol and 50 ng/μl ampicillin and inoculated to a liquid LB cultures with chloramphenicol and ampicillin. After overnight growth, 50 μl culture from each sample was plated on LB plates supplemented with 30 ng/μl kanamycin. After 16 h incubation at 37° C., colonies were counted. For experiments with more mutagenic TADR constructs as in FIG. 3A, less volumes of culture were plated as specified therein.

### Double Reversion Assay

It was the same kanR* reversion assay except that a chloramphenicol resistance gene took the place of kanR* and that it carried two stop codons (Q38stop, Y611stop) in the middle of the open reading frame. With the first stop codon, only 17% of the whole protein was expressed. The second stop codon truncated only nine amino acid residues but was known to inactivate chloramphenicol resistance(Robben et al., J Biol Chem, 268 (33): 24555-24558, (1993)). After induction in minimal medium, 50 pl culture of full density was plated onto LB supplemented with 15 ng/μl chlorampehnicol, and the plate was incubated at 37° C. for 16 hours before colonies were checked. These colonies were inoculated into liquid LB supplemented with chloramphenicol, and the target plasmids were purified and sent to Genewiz to Sanger-sequence the gene for chloramphenicol resistance. This assay tested the capacity of TADR to simultaneously discover two beneficial mutations of a protein in one round of evolution.

### Self-Evolution of TADR for Higher Mutation Rate

A schematic of the steps is shown in FIG. 6. The helper plasmid was modified so that the recognition sequenced was added at the 5′ UTR of the T5 DNA polymerase gene and that the termination sequence was added in the linker region as an extension of the linker without altering the sequences for T5 DNA polymerase and Rep helicase. This helper plasmid was transformed into TADR cells and plated on agar plate of minimal medium supplemented with 50 ng/μl ampicillin. After incubation at 37° C. for 24 h, a single colony was inoculated to liquid minimal medium with ampicillin to allow the T5 DNA polymerase gene to be mutagenized. After ˜24 h of growth, cells grew to mid-log, were treated for competency and electroporated with ˜500 ng of the target plasmid following standard protocol(Green, (2012)). Right after electro-pulsing, one ml SOC medium was added immediately to the 50 pl slurry of competent cells. This mix was incubated at 37° C. and shaken for five min before plated onto minimal medium plate supplemented with 15 ng/μl chloramphenicol, 50 ng/μl ampicillin and with 30 ng/μl kanamycin. After incubation at 37° C. for 16 h, typically a dozen colonies showed up, and the biggest a few were inoculated into liquid LB media supplemented with 15 ng/μl chloramphenicol and 50 ng/μl ampicillin. Not all of them would grow to full density; and for those did, plasmid mixes (of the helper and target) were purified and transformed into a fresh stock of the host cell for TADR and plated on LB plate with 30 ng/μl kanamycin. This step checked if the kanamycin resistance was carried on the target plasmid (true positive). For the plasmid mixes that gave rise to kanamycin resistant colonies and thus passed the check, they were treated by FastDigest PvuII (ThermoScientific), which only cut the target but not the helper plasmids, and purified using GeneJET Gel Extraction Kit. This treatment resulted in purified evolved helper plasmids, which were then subjected to the procedures of quantification of mutagenic capacity using kanR* reversion assay as described above. For the helper plasmids that showed increased capacity for mutagenesis compared to the ancestor, they were Sanger-sequenced (Genewiz) in the entire region of the fused gene to identify mutations.

### Characterization of Mutant Library Generated by TADR Using Next Generation Sequencing

TADR cells were transformed with the target plasmid with kanR* and the helper plasmid with RepΔC33, modified 5′ UTR and T5 DNA polymerase mutant 1308V (the most mutagenic version of TADR), and colonies were selected on minimal media supplemented with ampicillin and chloramphenicol. Wildtype E. coli cells (MG1655) were transformed with the target plasmid as negative control, and colonies were selected on minimal media supplemented with chloramphenicol. Three colonies from each transformation were separately grown up in minimal media with appropriate antibiotics to full density. Plasmids were purified. Six hundred ng plasmid from each sample were submitted to MiGS center in Pittsburgh for sequencing using Illumina NextSeq 550 with paired end reads of 150 base-pairs. Each sample was sequenced with the coverage of 20000 to 70000 to capture rare variants. Galaxy platform was used to process the data and map reads to the reference sequence of the target plasmid. The raw data was trimmed to filter out low-quality reads so that all reads had quality scores larger than 20. VarScan (version 2.4.4)(Koboldt et al., Genome Res, 22 (3): 568-576, (2012)) package was used to call variants. None of the variants from the three negative control samples exceeded 0.14% and thus 0.14% was used as a cutoff for point mutations. The three treatment samples contained a total of 264 point mutations above this cutoff value. These 264 point-mutation variants were further analyzed to plot mutation density across the target plasmid (FIG. 3D) and measure the mutational spectrum (FIG. 3E).

Evolution of tetA for Resistance to Tigecycline

The target plasmid with the tetA(C) gene was co-transformed with the helper plasmid carrying RepΔC33, modified 5′ UTR and mutation 1308V into the host cell for TADR. Transformant colonies were selected on LB agar plates with 15 ng/μl chloramphenicol and 50 ng/μl ampicillin and inoculated to a liquid LB culture with chloramphenicol and ampicillin. After overnight growth, 50 μl culture from each sample was plated on LB plates with 4 ng/μl tigecycline. After 16 h incubation at 37° C., several dozen colonies typically showed up, and the biggest a few were inoculated into liquid LB with chloramphenicol. After overnight culture, plasmids were purified, digested with FastDigest AflII (ThermoScientifc), which cut only the helper but not the target plasmids, and purified using GeneJET Gel Extraction Kit. This treatment resulted in purified evolved target plasmids. They were co-transformed with helper plasmid carrying modified 5′ UTR and mutation 1308V into the host cell for TADR for a second round of experimental evolution, and this time were selected on LB plates with 8 ng/μl tigecycline. The resulting plasmids were purified and Sanger-sequenced.

Five evolved plasmids were selected based on their big colony size on tigecycline plates during experimental evolution. Their tetA gene was each PCR-amplified and inserted into the backbone of the unevolved target plasmid to purge any serendipitous off-target mutations accumulated during evolution. The resulting reconstructed evolved plasmids were transformed into the host cell for TADR, and the growth rates were measured in the absence of antibiotic, in tetracycline and in tigecycline using SpectraMax Plus 384 Microplate Reader (200 l volume, OD 600, 37° C., shaken for 500 s followed by 100 s still continuously, one reading every 600 s, 16 h in total; Molecular Devices). The readings of OD 600 were analyzed using JMP software (SAS): for the growth curve of each sample, the optical density values were log-transformed, and slope of the linear part was calculated as the corresponding growth rate.

### Choice of DNA Polymerase

Motivated by single-molecule studies (Maier et al., Proceedings of the National Academy of Sciences, 97 (22): 12002-12007, (2000); Arslan et al., Science (New York, N.Y.), 348 (6232): 344-347, (2015)), TADR was initially implemented with T7 DNA polymerase because its speed profile in relation to that of Rep helicase predicted a processive and functional replisome (FIG. 1C). However, no targeted mutagenesis was seen as tested using kanR* reversion assay as per FIG. 2C. It was noted that the C-terminal residue (H704) of T7 DNA polymerase, which in TADR was linked to the linker through peptide bond, happened to be important for the catalysis of polymerization (Kumar et al., J Biol Chem, 276 (37): 34905-34912, (2001)), and that this positively charged residue was conserved among many DNA polymerases (Table 3). One study found that linking a six-His tag (SEQ ID NO: 37) to this residue of DNA polymerase I from bacterium Streptococcus pneumoniae reduced polymerase activity five-fold (Amblar et al., Journal of Biological Chemistry, 276 (22): 19172-19181, (2001)). It was reasoned that anchoring the linker to this catalytic residue of T7 DNA polymerase might have also hindered the function of the polymerase, leading to the failure of this attempt.

T5 DNA polymerase provided a promising alternative. It had an extra domain after the catalytic residue (Table 3). This domain was tested to be unimportant to polymerase activity(Andraos et al., Journal of Biological Chemistry, 279 (48): 50609-50618, (2004)), and linking a six-His tag (SEQ ID NO: 37) to its C-terminus did not interfere with polymerase activity (Andraos et al., Journal of Biological Chemistry, 279 (48): 50609-50618, (2004)). Hence, this polymerase was compatible with a linker to its C-terminus. Also, this polymerase was known to be processive with a speed of DNA synthesis comparable to that of T7 DNA polymerase. This high speed was important because some polymerases such as E. coli PolA had a speed of ˜15 base-pairs/s (Maier et al., Proceedings of the National Academy of Sciences, 97 (22): 12002-12007, (2000)), lower than that of Rep helicase (144 base-pairs/s). In the case of a slow DNA polymerase, it would not catch up with Rep helicase but lag behind with the newly unwound single-strand DNA building up between the two proteins. This excessive single-strand DNA would be degraded by exonucleases abundant in cells, destroying the target DNA (Lovett, EcoSal Plus, 4 (2): 10.1128/ecosalplus.1124.1124.1127, (2011)). Hence, the high speed of T5 DNA polymerase (at least higher than that of Rep helicase) was critical to the scenario depicted in FIG. 1C that favored a processive and functional replisome.

Toxicity of the Target Plasmid without Initiation Sequence

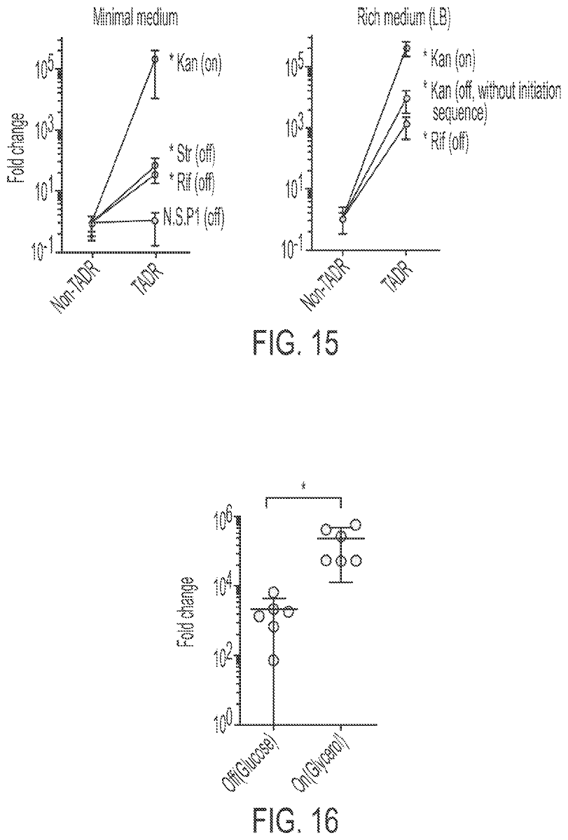

It was noted that cells from the control of the target plasmid without initiation sequence grew substantially slower in LB than those from the treatment (with initiation sequence), regardless of Rep genotype. In minimal media, this pattern was more pronounced: cells from the control did not grow at all while cells from the treatment grew normally. A potential explanation was that expression of the fusion protein might be toxic to the cell but this toxicity might be diminished with titrating the fusion protein by binding to the target plasmid with initiation sequence. Consistent with this scenario of titration was another observation that mutations of TARD with RepΔC33 were higher than those with wildtype Rep (FIG. 2c, third column, black dots v.s. gray dots; ANOVA, n=5, P<0.0001). That is, while wildtype Rep directed the error-prone T5 DNA polymerase to both the chromosome and target plasmid, RepΔC33 directed it to the target plasmid only, accumulating more mutations here. Also consistent with the scenario of titration, the genetic control whose target plasmid was without an initiation sequence measured in LB a higher off-target mutagenic activity than the physiological control using spontaneous resistance to rifampicin (FIG. 15).

### Adaptive Synonymous Mutations

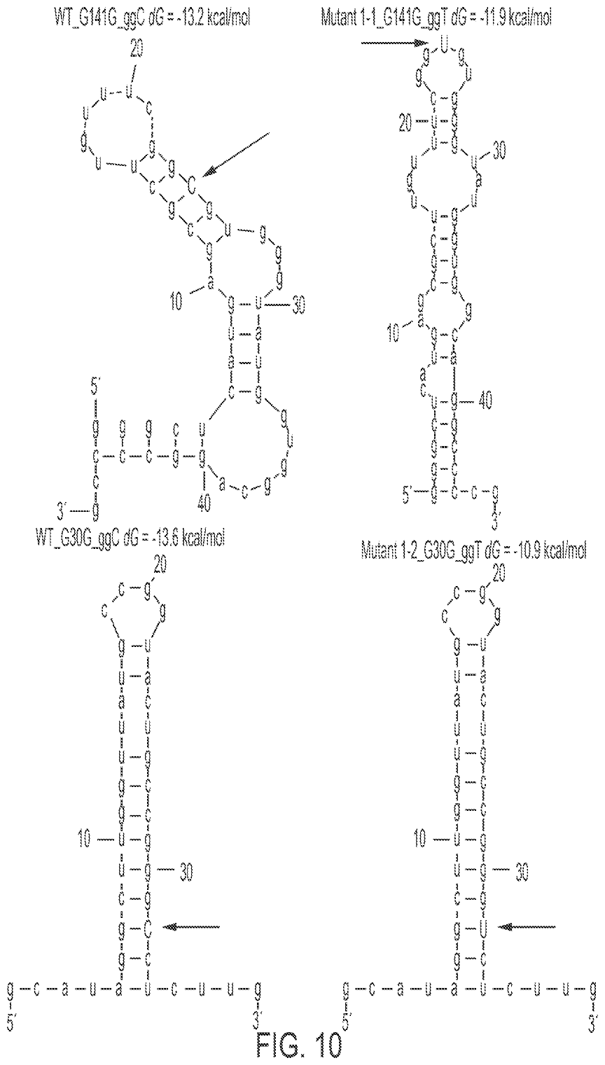

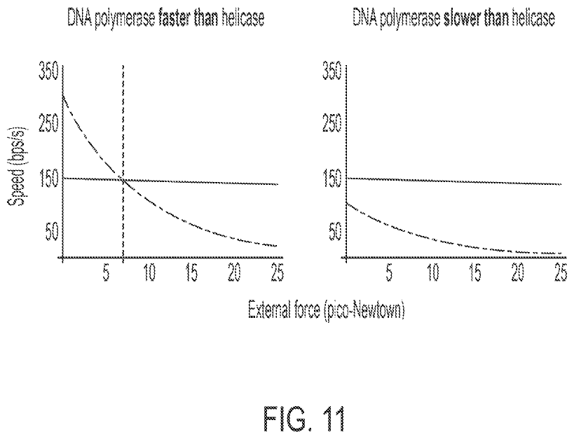

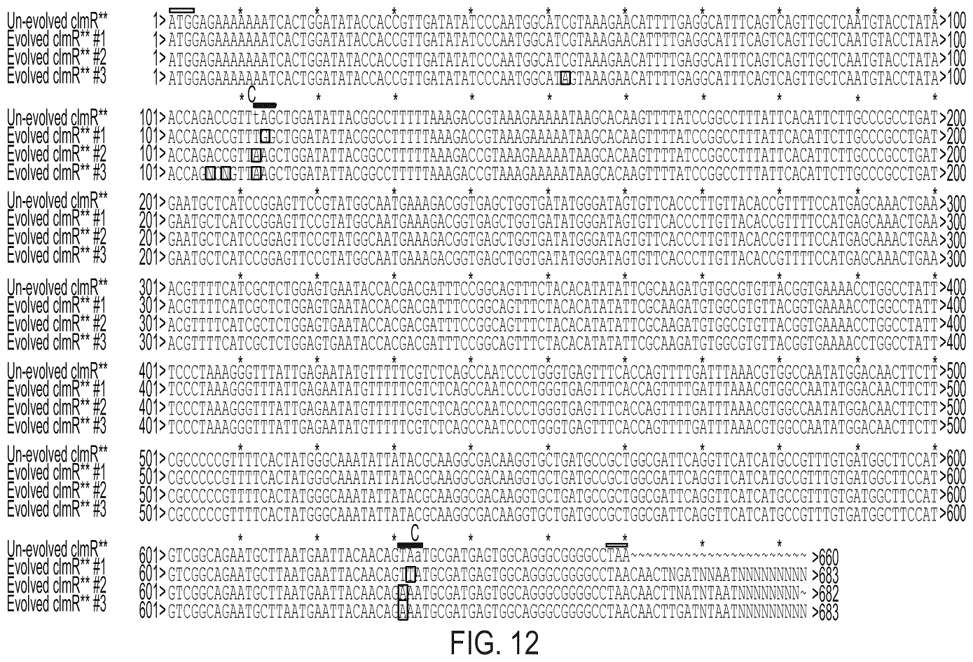

Of the five evolved tetA mutants characterized, three carried synonymous mutations (Table 2). Two (Mutants 1-1, 1-2) clearly increased growth rate and/or antibiotic resistance (FIG. 9) and thus were adaptive in that they evolved from Mutant 1 which did not carry these synonymous mutations. In both cases, it was found that the mRNA transcripts around the mutated residues had substantial secondary structures (FIG. 10) and that the substitutions destabilized the structures by reducing the free energies of the structures. Hence, these synonymous mutations might have reduced the cost of expressing the efflux pump, thereby increasing growth rates both in the presence and absence of antibiotics.

### NGS Identifies Mutational Hotspots Explicable by Molecular Collision

A closer examination of the mutational profile finds that the major hotspot in FIG. 3D consists of two peaks. Beside the one at the promoter for the gene(s) of interest, a second sits inside origin of replication (ORI) and happens to precisely overlap with the promoter for the initiation of plasmid replication (FIG. 17). Now all the mutational hotspots coincide with transcription initiation and termination.

Machineries of transcription and DNA replication are known to collide with each other in cells (Pomerantz, et al., Cell Cycle 9, 2537-2543 (2010)). From this knowledge emerges a scenario of molecular collision-induced hypermutagenesis. TADR replisome frequently reads through the termination sequence, which explains why increased mutagenesis is seen throughout the target plasmid. This read-though opens the chances that TADR replisome collides with RNA polymerase. To initiate transcription, RNA polymerase first sits at the promoter waiting for a substantial amount of time until a favorable kick from thermodynamic noise to set it off (Meng, et al., Nat. Commun. 8, 1178 (2017)). Hence, it is highly likely that TADR replisome collides with RNA polymerase waiting at the promoters and stalls from the collision (Labib, et al., EMBO Rep. 8, 346-353 (2007)). T5 DNA polymerase also stalls. Importantly, both experiment and theory have shown that stalling DNA polymerase increases error rate (Murat, et al., Genome Biol. 21, 209 (2020) and Banerjee, et al., PNAS 114, 5183-5188 (2017)). Then stalling T5 DNA polymerase by the sitting RNA polymerase leads to increased mutagenesis, explaining the major mutational hotspots. The minor hotspot at the site of transcription termination is explained similarly: a cruising RNA polymerase pauses at the site of transcriptional termination and waits for some time before falling off the plasmid (Kang, et al., Nat. Commun. 11, 450 (2020)). In the meantime, a TADR replisome runs into it and stalls at the same site, causing increased mutagenesis here.

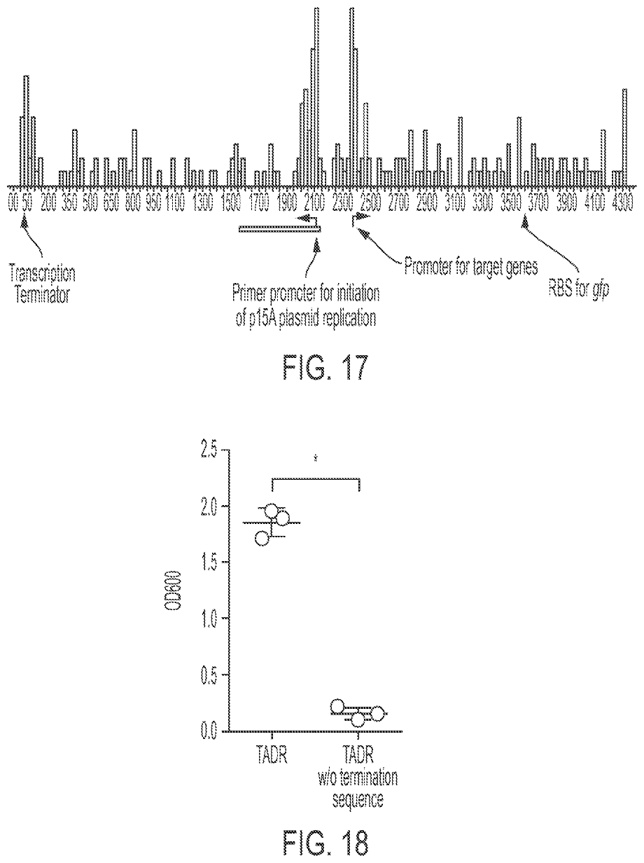

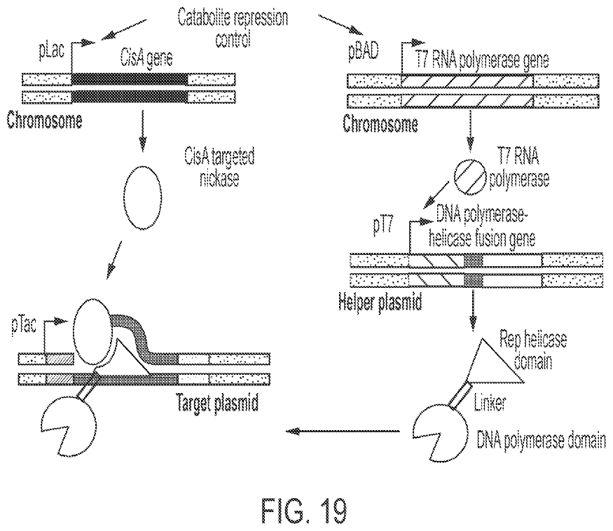

The linchpin of the scenario above is the collisions. Experimental test of the collisions is needed. Prior work showed that once the cisA/Rep complex bound to a plasmid at the initiation sequence (absent termination sequence and any other molecular motors), it rolled around the plasmid circle 15 times before dissociating (Eisenberg, et al., PNAS. 74, 3198-3202 (1977)). This stable and enduring operation of the cisA/Rep complex on the plasmid would ensure that the complex collides with other molecular motors operating on the same plasmid when these motors are present. A construct was made herein to test this prediction: the target plasmid was modified to remove the termination sequence, mimicking the scenario of the prior work (Eisenberg, et al., PNAS. 74, 3198-3202 (1977)). Consistent with our prediction: TADR cells carrying this modified target plasmid and the helper plasmid failed to grow up in the presence of the antibiotic selecting for the target plasmid (FIG. 18). This result is expected because the now unchained complex of cisA/Rep in TADR incessantly occupies the target plasmid, constantly colliding with some early-stage protein for replication initiation, such as RNase H (Selzer, et al., PNAS. 79, 7082-7086 (1982)), once the protein binds at ORI. Binding by this protein is known to be weak (Lima, et al., Biochemistry 36, 390-398 (1997)) and thus easily disrupted by the collision, thereby preventing initiation of plasmid replication, letting the antibiotic kill the cell. To the contrary, with presence of the termination sequence, TADR replisome stops at it 43% of the time (see Result section “NGS confirms the designed mechanisms for high performance of TADR”), leaving opportunity for completing the initiation of plasmid replication. As a result, an innate replisome starts to replicate the plasmid, and its tight binding to DNA precludes TADR replisome from disrupting the replication process. In conclusion, molecular collision-induced hypermutagenesis accounts for the observations.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

TABLE 1
Comparison of different 5′ ends of the fused gene in their translation strength.
The translation strength was predicted based on thermodynamic calculations in the webserver of
RBS Calculator (Espah Borujeni et al., Nucleic Acids Research, 45 (9): 5437-5448, (2017)). The
start codon is shown bold; ribosomal binding site, italicized; the recognition sequence of TADR,
underlined.

| | Predicted | |
|---|---|---|
| Versions of | translation strength | |
| helper plasmid | (a.u.) | 5′ end of the transcript sequence |
| Original nonself | 37 | ggcucuauccagguuauaaaugaaaaucgcaguaguuga |
| | | (SEQ ID NO: 42) |
| Original self | 367 | ggcucuaucccaacuugauauuaauaacacuauagaccacagg |
| | | uuauaaaugaaaaucgcaguaguuga (SEQ ID NO: 43) |
| 5′ UTR modified | 13832 | ggcucuauccagaagttatagtgttattaatattaggttgagguuaua |
| | | aaugaaaaucgcaguaguuga (SEQ ID NO: 44) |

TABLE 2
Mutations in evolved tetA efflux pumps. Amino acid
substitutions are given. For synonymous mutations,
the nucleotide substitutions are given.

| Index | Mutations in tetA |
|---|---|
| 1 | I235F, S312F |
| 1-1 | G141G (ggc > ggt), I235F, S312F |
| 1-2 | G30G (ggc > ggt), I235F, S312F |
| 2 | I235F, A370V |
| 3 | I235F, I248N, I308I (att > atc) |

TABLE 3
Multiple sequence alignments highlight the unusual extra domain of T5 DNA
polymerase at C-terminus. The conserved C-terminal catalytic residues were bolded. Italicized
letters mark the extra domain unique to T5 DNA polymerase. Table discloses SEQ ID NOS: 33-
36, respectively, in order of appearance.

| | | |
|---|---|---|
| T7 coliphage Pol | QVVI---ETAQEAMRWVGDHWNFRCLLDTEGKMGPNWAICH------------------- | 704 |
| T5 coliphage Pol | QYNEILIRNIQKDRGISIPGCPIGIDSDSEAGGSRDYSCGKMKKQHPSIACIDDDEYTRY | 806 |
| E. coli PolA | AVAKQIHQLMENC-------TRLDVPLLVEVGSGENWDQAH------------------- | 928 |
| T. aquaticus PolA | AVARLAKEVMEGV-------YPLAVPLEVEVGIGEDWLSAKE------------------ | 832 |
| T7 coliphage Pol | ------------------------------------------------- | 704 |
| T5 coliphage Pol | VKGVLLDAEFEYKKLAAMDKEHPDHSKYKDDKFIAVCKDLDNVKRILGA | 855 |
| E. coli PolA | -------------------------------------------------- | 928 |
| T. aquaticus PolA | -------------------------------------------------- | 832 |

TABLE 4
Determinants of mutation rate and targeting selectivity of
TADR, and their corresponding strategies for optimization.
The measured effects from applying these strategies are also
given. N/A, not applicable. CisA has evolved in nature to
bind initiation sequence tightly with high specificity.

| | | Optimization | Effects (fold |
|---|---|---|---|
| | Determinants | strategies | increase) |
| Mutation | Expression level | Self-evolve TADR | 3.2 |
| rate | of T5 DNA | to increase error | |
| | polymerase | rate | |
| | Expression level | Modify 5′ UTR to | 150 |
| | of the fusion | increase | |
| | protein | transcription of | |
| | | the fusion gene | |
| Targeting | Binding affinity | N/A | |
| selectivity | of cisA to | | |
| | initiation | | |
| | sequence | | |
| | Rep helicase and | Adopt RepAC33 | 16 |
| | thus its fused | mutant to | |
| | error-prone T5 | eliminate | |
| | DNA polymerase | colocalization to | |
| | colocalize with | reduce off-target | |
| | innate replisome, | mutations | |
| | causing off-target | | |
| | mutations | | |
| | Encounter of error- | Reduce | 89, 45* |
| | prone T5 DNA | concentration | |
| | polymerase with | (copy number per | |
| | innate replisome | cell) of | |
| | by random | chromosome and | |
| | diffusion, causing | thus innate | |
| | off-target | replisome by | |
| | mutations | growing cells in | |
| | | minimal medium | |
| | | to reduce off- | |
| | | target mutations | |

*Off-target mutation rate used to calculate selectivity was measured by spontaneous rifampicin (89) or streptomycin (45) resistance.

TABLE 5
Informal Sequence Listing

| | |
|---|---|
| SEQ | |
| ID | |
| NO. | Embodiments of Sequences |

Embodiments of T5 DNA Polymerase Amino Acid Sequences

| | |
|---|---|
| 1 | >YP_006950.1 DNA polymerase [Escherichia virus T5] |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFDSETSALYCRDGYLLGVSISHQEYQGVYIDSDCLTEVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDIMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLFPALRQAAKAITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGA |
| 2 | > one embodiment of T5 DNA polymerase mutant (D164A, E166A, A593R) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTEVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDIMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLEPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGA |
| 3 | > one embodiment of T5 DNA polymerase mutant (D164A, E166A, I308V, A593R) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTEVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDVMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLEPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGA |
| 4 | > one embodiment of T5 DNA polymerase mutant (I308V) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFDSETSALYCRDGYLLGVSISHQEYQGVYIDSDCLTEVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDVMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLFPALRQAAKAITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGA |
| 26 | >one embodiment of nucleic acid encoding T5 DNA polymerase mutant |
| | (D164A, E166A, I308V, A593R) |
| | ATGAAAATCGCAGTAGTTGATAAAGCTCTAAACAACACTCGTTATGATAAACATTTCCAGCTATACGGCG |
| | AGGAAGTTGATGTATTCCATATGTGTAACGAGAAGTTGTCCGGTCGTTTGCTCAAAAAGCATATTACTAT |
| | CGGAACTCCGGAAAACCCATTTGACCCGAATGATTATGATTTTGTTATACTGGTAGGTGCCGAACCTTTC |
| | CTGTACTTTGCAGGTAAGAAAGGTATTGGTGATTATACCGGTAAACGTGTAGAGTATAATGGATATGCTA |
| | ACTGGATTGCGAGTATCAGCCCAGCCCAGTTACACTTTAAACCTGAAATGAAACCAGTTTTTGATGCAAC |
| | AGTAGAGAATATCCACGATATTATCAATGGTCGTGAGAAGATTGCAAAAGCTGGTGATTACCGTCCTATT |
| | ACTGACCCTGATGAGGCTGAAGAATATATCAAGATGGTGTATAATATGGTTATCGGACCCGTCGCATTCG |
| | CCTCCGCAACCTCAGCACTATACTGTCGAGATGGTTATCTGCTTGGTGTTTCTATTTCTCACCAAGAGTA |
| | TCAGGGTGTATATATCGATTCTGATTGTCTCACAGAGGTTGCAGTATATTATCTCCAGAAAATTCTGGAT |
| | AGTGAAAACCACACTATTGTTTTTCACAACTTGAAGTTTGATATGCACTTTTATAAGTACCATCTGGGAC |
| | TTACTTTTGATAAAGCACATAAAGAACGCAGGCTCCATGATACCATGTTGCAGCACTATGTTTTAGATGA |
| | ACGTCGTGGTACTCATGGCTTGAAATCTCTAGCAATGAAGTATACCGATATGGGTGACTATGACTTCGAA |
| | CTAGATAAGTTCAAAGATGATTACTGTAAAGCACATAAAATCAAGAAAGAAGATTTCACCTATGATTTAA |
| | TTCCGTTTGATGTTATGTGGCCATATGCTGCGAAAGATACGGATGCCACTATACGTTTGCACAACTTCTT |
| | TTTACCAAAAATTGAGAAGAATGAAAAACTTTGCAGTCTGTATTACGATGTTTTGATGCCTGGTTGCGTA |
| | TTCTTGCAACGTGTTGAGGATCGTGGAGTACCTATCTCTATTGATCGTTTGAAAGAAGCTCAGTATCAGT |
| | TGACTCATAATTTGAATAAAGCCCGTGAGAAACTGTACACTTATCCAGAAGTTAAACAGCTAGAACAAGA |
| | TCAGAATGAAGCATTTAACCCGAACTCTGTTAAGCAGCTACGTGTTCTTCTGTTTGATTACGTTGGCTTA |
| | ACTCCAACAGGTAAACTGACGGATACTGGAGCAGATTCTACGGATGCAGAAGCTCTAAATGAACTGGCTA |
| | CGCAGCATCCAATTGCTAAAACTCTGCTAGAGATTCGTAAGCTGACTAAGCTGATCTCTACTTATGTTGA |
| | GAAGATTCTACTGAGTATTGATGCAGATGGTTGCATTCGTACTGGTTTCCATGAACATATGACTACTTCT |
| | GGTCGTCTGAGTTCTTCTGGTAAACTGAACCTGCAACAGTTACCCCGTGATGAATCTATTATCAAGGGTT |
| | GTGTAGTAGCTCCTCCTGGGTATCGTGTAATCGCGTGGGACTTAACAACTGCGGAAGTTTATTATGCTGC |
| | TGTTCTATCTGGTGATAGAAATATGCAACAGGTATTTATCAACATGAGAAATGAACCCGATAAATACCCA |
| | GACTTCCACTCCAACATCGCACACATGGTGTTTAAGCTGCAATGCGAACCCCGTGATGTTAAAAAGCTGT |
| | TCCCAGCTCTGCGTCAGGCTGCTAAACGCATCACCTTCGGTATTCTGTATGGTTCTGGCCCAGCTAAAGT |
| | AGCGCATTCTGTTAACGAAGCATTACTAGAACAAGCAGCCAAGACGGGCGAACCGTTTGTTGAATGTACT |
| | GTTGCAGATGCTAAAGAGTACATTGAGACTTACTTCGGTCAGTTCCCACAGCTTAAGCGTTGGATTGATA |
| | AGTGCCACGATCAGATCAAGAATCATGGATTTATCTATAGTCACTTTGGTCGTAAACGTCGTCTGCATAA |
| | TATCCATTCCGAAGACCGTGGTGTTCAGGGTGAAGAAATTCGTTCTGGATTTAATGCAATCATTCAGTCT |
| | GCTTCTTCTGATAGTCTCCTTTTAGGTGCTGTAGATGCAGATAATGAGATCATTTCTCTTGGTTTAGAAC |
| | AAGAGATGAAGATTGTTATGTTGGTTCATGACTCCGTAGTTGCTATTGTTCGTGAGGATTTGATCGACCA |
| | ATACAATGAAATCCTGATTCGTAATATTCAGAAAGACCGTGGTATCAGTATTCCTGGCTGTCCGATTGGT |
| | ATTGATTCAGATTCTGAAGCTGGAGGTTCTCGTGACTATTCTTGTGGTAAGATGAAGAAACAGCACCCAT |
| | CAATCGCTTGTATTGATGATGATGAATATACTCGTTATGTCAAGGGTGTATTACTTGATGCAGAATTCGA |
| | GTATAAGAAACTAGCTGCAATGGATAAAGAGCATCCAGATCATAGCAAATACAAGGATGATAAGTTTATT |
| | GCTGTATGTAAAGATTTGGATAACGTGAAAAGGATTCTCGGTGCT |
| 29 | >one embodiment of nucleic acid encoding T5 DNA polymerase (wild type) |
| | ATGAAAATCGCAGTAGTTGATAAAGCTCTAAACAACACTCGTTATGATAAACATTTCCAGCTATACGGCG |
| | AGGAAGTTGATGTATTCCATATGTGTAACGAGAAGTTGTCCGGTCGTTTGCTCAAAAAGCATATTACTAT |
| | CGGAACTCCGGAAAACCCATTTGACCCGAATGATTATGATTTTGTTATACTGGTAGGTGCCGAACCTTTC |
| | CTGTACTTTGCAGGTAAGAAAGGTATTGGTGATTATACCGGTAAACGTGTAGAGTATAATGGATATGCTA |
| | ACTGGATTGCGAGTATCAGCCCAGCCCAGTTACACTTTAAACCTGAAATGAAACCAGTTTTTGATGCAAC |
| | AGTAGAGAATATCCACGATATTATCAATGGTCGTGAGAAGATTGCAAAAGCTGGTGATTACCGTCCTATT |
| | ACTGACCCTGATGAGGCTGAAGAATATATCAAGATGGTGTATAATATGGTTATCGGACCCGTCGCATTCG |
| | ACTCCGAAACCTCAGCACTATACTGTCGAGATGGTTATCTGCTTGGTGTTTCTATTTCTCACCAAGAGTA |
| | TCAGGGTGTATATATCGATTCTGATTGTCTCACAGAGGTTGCAGTATATTATCTCCAGAAAATTCTGGAT |
| | AGTGAAAACCACACTATTGTTTTTCACAACTTGAAGTTTGATATGCACTTTTATAAGTACCATCTGGGAC |
| | TTACTTTTGATAAAGCACATAAAGAACGCAGGCTCCATGATACCATGTTGCAGCACTATGTTTTAGATGA |
| | ACGTCGTGGTACTCATGGCTTGAAATCTCTAGCAATGAAGTATACCGATATGGGTGACTATGACTTCGAA |
| | CTAGATAAGTTCAAAGATGATTACTGTAAAGCACATAAAATCAAGAAAGAAGATTTCACCTATGATTTAA |
| | TTCCGTTTGATATTATGTGGCCATATGCTGCGAAAGATACGGATGCCACTATACGTTTGCACAACTTCTT |
| | TTTACCAAAAATTGAGAAGAATGAAAAACTTTGCAGTCTGTATTACGATGTTTTGATGCCTGGTTGCGTA |
| | TTCTTGCAACGTGTTGAGGATCGTGGAGTACCTATCTCTATTGATCGTTTGAAAGAAGCTCAGTATCAGT |
| | TGACTCATAATTTGAATAAAGCCCGTGAGAAACTGTACACTTATCCAGAAGTTAAACAGCTAGAACAAGA |
| | TCAGAATGAAGCATTTAACCCGAACTCTGTTAAGCAGCTACGTGTTCTTCTGTTTGATTACGTTGGCTTA |
| | ACTCCAACAGGTAAACTGACGGATACTGGAGCAGATTCTACGGATGCAGAAGCTCTAAATGAACTGGCTA |
| | CGCAGCATCCAATTGCTAAAACTCTGCTAGAGATTCGTAAGCTGACTAAGCTGATCTCTACTTATGTTGA |
| | GAAGATTCTACTGAGTATTGATGCAGATGGTTGCATTCGTACTGGTTTCCATGAACATATGACTACTTCT |
| | GGTCGTCTGAGTTCTTCTGGTAAACTGAACCTGCAACAGTTACCCCGTGATGAATCTATTATCAAGGGTT |
| | GTGTAGTAGCTCCTCCTGGGTATCGTGTAATCGCGTGGGACTTAACAACTGCGGAAGTTTATTATGCTGC |
| | TGTTCTATCTGGTGATAGAAATATGCAACAGGTATTTATCAACATGAGAAATGAACCCGATAAATACCCA |
| | GACTTCCACTCCAACATCGCACACATGGTGTTTAAGCTGCAATGCGAACCCCGTGATGTTAAAAAGCTGT |
| | TCCCAGCTCTGCGTCAGGCTGCTAAAGCAATCACCTTCGGTATTCTGTATGGTTCTGGCCCAGCTAAAGT |
| | AGCGCATTCTGTTAACGAAGCATTACTAGAACAAGCAGCCAAGACGGGCGAACCGTTTGTTGAATGTACT |
| | GTTGCAGATGCTAAAGAGTACATTGAGACTTACTTCGGTCAGTTCCCACAGCTTAAGCGTTGGATTGATA |
| | AGTGCCACGATCAGATCAAGAATCATGGATTTATCTATAGTCACTTTGGTCGTAAACGTCGTCTGCATAA |
| | TATCCATTCCGAAGACCGTGGTGTTCAGGGTGAAGAAATTCGTTCTGGATTTAATGCAATCATTCAGTCT |
| | GCTTCTTCTGATAGTCTCCTTTTAGGTGCTGTAGATGCAGATAATGAGATCATTTCTCTTGGTTTAGAAC |
| | AAGAGATGAAGATTGTTATGTTGGTTCATGACTCCGTAGTTGCTATTGTTCGTGAGGATTTGATCGACCA |
| | ATACAATGAAATCCTGATTCGTAATATTCAGAAAGACCGTGGTATCAGTATTCCTGGCTGTCCGATTGGT |
| | ATTGATTCAGATTCTGAAGCTGGAGGTTCTCGTGACTATTCTTGTGGTAAGATGAAGAAACAGCACCCAT |
| | CAATCGCTTGTATTGATGATGATGAATATACTCGTTATGTCAAGGGTGTATTACTTGATGCAGAATTCGA |
| | GTATAAGAAACTAGCTGCAATGGATAAAGAGCATCCAGATCATAGCAAATACAAGGATGATAAGTTTATT |
| | GCTGTATGTAAAGATTTGGATAACGTGAAAAGGATTCTCGGTGCT |

Embodiments of Rep helicase Amino Acid Sequences

| | |
|---|---|
| 5 | > DNA helicase Rep [Enterobacteriaceae] |
| | RLNPGQQQAVEFVTGPCLVLAGAGSGKTRVITNKIAHLIRGCGYQARHIAAVTFTNKAAREMKERVGQT |
| | LGRKEARGLMISTFHTLGLDIIKREYAALGMKANFSLFDDTDQLALLKELTEGLIEDDKVLLQQLISTIS |
| | NWKNDLKTPSQAAASAIGERDRIFAHCYGLYDAHLKACNVLDFDDLILLPTLLLQRNEEVRKRWQNKIRY |
| | LLVDEYQDTNTSQYELVKLLVGSRARFTVVGDDDQSIYSWRGARPQNLVLLSQDFPALKVIKLEQNYRSS |
| | GRILKAANILIANNPHVFEKRLFSELGYGAELKVLSANNEEHEAERVTGELIAHHFVNKTQYKDYAILYR |
| | GNHQSRVFEKFLMQNRIPYKISGGTSFFSRPEIKDLLAYLRVLTNPDDDSAFLRIVNTPKREIGPATLKK |
| | LGEWAMTRNKSMFTASFDMGLSQTLSGRGYEALTRFTHWLAEIQRLAEREPIAAVRDLIHGMDYESWLYE |
| | TSPSPKAAEMRMKNVNQLFSWMTEMLEGSELDEPMTLTQVVTRFTLRDMMERGESEEELDQVQLMTLHAS |
| | KGLEFPYVYMVGMEEGFLPHQSSIDEDNIDEERRLAYVGITRAQKELTFTLCKERRQYGELVRPEPSRFL |
| | LELPQDDLIWEQERKVVSAEERMQKGQSHLANLKAMMAAKRGK |
| 20 | >one embodiment of DNA helicase Rep (truncated mutant) |
| | RLNPGQQQAVEFVTGPCLVLAGAGSGKTRVITNKIAHLIRGCGYQARHIAAVTFTNKAAREMKERVGQT |
| | LGRKEARGLMISTFHTLGLDIIKREYAALGMKANFSLFDDTDQLALLKELTEGLIEDDKVLLQQLISTIS |
| | NWKNDLKTPSQAAASAIGERDRIFAHCYGLYDAHLKACNVLDFDDLILLPTLLLQRNEEVRKRWQNKIRY |
| | LLVDEYQDTNTSQYELVKLLVGSRARFTVVGDDDQSIYSWRGARPQNLVLLSQDFPALKVIKLEQNYRSS |
| | GRILKAANILIANNPHVFEKRLFSELGYGAELKVLSANNEEHEAERVTGELIAHHFVNKTQYKDYAILYR |
| | GNHQSRVFEKFLMQNRIPYKISGGTSFFSRPEIKDLLAYLRVLTNPDDDSAFLRIVNTPKREIGPATLKK |
| | LGEWAMTRNKSMFTASFDMGLSQTLSGRGYEALTRFTHWLAEIQRLAEREPIAAVRDLIHGMDYESWLYE |
| | TSPSPKAAEMRMKNVNQLFSWMTEMLEGSELDEPMTLTQVVTRFTLRDMMERGESEEELDQVQLMTLHAS |
| | KGLEFPYVYMVGMEEGFLPHQSSIDEDNIDEERRLAYVGITRAQKELTFTLCKERRQYGELVRPEPSRFL |
| | LELPQDDLIW |
| 27 | >one embodiment of nucleic acid encoding DNA helicase Rep (truncated |
| | mutant) |
| | CGTCTAAACCCCGGCCAACAACAAGCTGTCGAATTCGTTACCGGCCCCTGCCTGGTGCTGGCGGGCGCGG |
| | GTTCCGGTAAAACTCGTGTTATCACCAATAAAATCGCCCATCTGATCCGCGGTTGCGGTTATCAGGCGCG |
| | GCACATTGCGGCGGTGACCTTTACTAATAAAGCAGCGCGCGAGATGAAAGAGCGTGTAGGGCAGACGCTG |
| | GGGCGCAAAGAGGCGCGTGGGCTGATGATCTCCACTTTCCATACGTTGGGGCTGGATATCATCAAACGCG |
| | AGTATGCGGCGCTTGGGATGAAAGCGAACTTCTCGTTGTTTGACGATACCGATCAGCTTGCTTTGCTTAA |
| | AGAGTTGACCGAGGGGCTGATTGAAGATGACAAAGTTCTCCTGCAACAACTGATTTCGACCATCTCTAAC |
| | TGGAAGAATGATCTCAAAACACCGTCCCAGGCGGCAGCAAGTGCGATTGGCGAGCGGGACCGTATTTTTG |
| | CCCATTGTTATGGGCTGTATGATGCACACCTGAAAGCCTGTAACGTTCTCGACTTCGATGATCTGATTTT |
| | ATTGCCGACGTTGCTGCTGCAACGCAATGAAGAAGTCCGCAAGCGCTGGCAGAACAAAATTCGCTATCTG |
| | CTGGTGGATGAGTATCAGGACACCAACACCAGCCAGTATGAGCTGGTGAAACTGCTGGTGGGCAGCCGCG |
| | CGCGCTTTACCGTGGTGGGTGACGATGACCAGTCGATCTACTCCTGGCGCGGTGCACGTCCGCAAAACCT |
| | GGTGCTGCTGAGTCAGGATTTTCCGGCGCTGAAGGTGATTAAGCTTGAGCAGAACTATCGCTCTTCCGGG |
| | CGTATTCTGAAAGCGGCGAACATCCTGATCGCCAATAACCCGCACGTCTTTGAAAAGCGTCTGTTCTCCG |
| | AACTGGGTTATGGCGCGGAGCTAAAAGTATTAAGCGCGAATAACGAAGAACATGAGGCTGAGCGCGTTAC |
| | TGGCGAGCTGATCGCCCATCACTTCGTCAATAAAACGCAGTACAAAGATTACGCCATTCTTTATCGCGGT |
| | AACCATCAGTCGCGGGTGTTTGAAAAATTCCTGATGCAAAACCGCATCCCGTACAAAATATCTGGTGGTA |
| | CGTCGTTTTTCTCTCGTCCTGAAATCAAGGACTTGCTGGCTTATCTGCGCGTGCTGACTAACCCGGACGA |
| | TGACAGCGCATTTCTGCGTATCGTTAACACGCCGAAGCGAGAGATTGGCCCGGCTACGCTGAAAAAGCTG |
| | GGTGAGTGGGCGATGACGCGCAATAAAAGCATGTTTACCGCCAGCTTTGATATGGGCCTGAGTCAGACGC |
| | TTAGCGGACGTGGTTATGAAGCATTGACCCGCTTCACTCACTGGTTGGCAGAAATCCAGCGTCTGGCGGA |
| | GCGGGAGCCGATTGCCGCGGTGCGTGATCTGATCCATGGCATGGATTATGAATCCTGGCTGTACGAAACA |
| | TCGCCCAGCCCGAAAGCCGCCGAAATGCGCATGAAGAACGTCAACCAACTGTTTAGCTGGATGACGGAGA |
| | TGCTGGAAGGCAGTGAACTGGATGAGCCGATGACGCTCACCCAGGTGGTGACGCGCTTTACTTTGCGCGA |
| | CATGATGGAGCGTGGTGAGAGTGAAGAAGAGCTGGATCAGGTGCAACTGATGACTCTCCACGCGTCGAAA |
| | GGGCTGGAGTTTCCTTATGTCTACATGGTCGGTATGGAAGAAGGGTTTTTGCCGCACCAGAGCAGCATCG |
| | ATGAAGATAATATCGATGAGGAGCGGCGGCTGGCCTATGTCGGCATTACCCGCGCCCAGAAGGAATTGAC |
| | CTTTACGCTGTGTAAAGAACGCCGTCAGTACGGCGAACTGGTGCGCCCGGAGCCGAGCCGCTTTTTGCTG |
| | GAGCTGCCGCAGGATGATCTGATTTGGTAA |
| 28 | >one embodiment of nucleic acid encoding DNA helicase Rep (full |
| | length) |
| | CGTCTAAACCCCGGCCAACAACAAGCTGTCGAATTCGTTACCGGCCCCTGCCTGGTGCTGGCGGGCG |
| | CGGGTTCCGGTAAAACTCGTGTTATCACCAATAAAATCGCCCATCTGATCCGCGGTTGCGGTTATCAGGC |
| | GCGGCACATTGCGGCGGTGACCTTTACTAATAAAGCAGCGCGCGAGATGAAAGAGCGTGTAGGGCAGACG |
| | CTGGGGCGCAAAGAGGCGCGTGGGCTGATGATCTCCACTTTCCATACGTTGGGGCTGGATATCATCAAAC |
| | GCGAGTATGCGGCGCTTGGGATGAAAGCGAACTTCTCGTTGTTTGACGATACCGATCAGCTTGCTTTGCT |
| | TAAAGAGTTGACCGAGGGGCTGATTGAAGATGACAAAGTTCTCCTGCAACAACTGATTTCGACCATCTCT |
| | AACTGGAAGAATGATCTCAAAACACCGTCCCAGGCGGCAGCAAGTGCGATTGGCGAGCGGGACCGTATTT |
| | TTGCCCATTGTTATGGGCTGTATGATGCACACCTGAAAGCCTGTAACGTTCTCGACTTCGATGATCTGAT |
| | TTTATTGCCGACGTTGCTGCTGCAACGCAATGAAGAAGTCCGCAAGCGCTGGCAGAACAAAATTCGCTAT |
| | CTGCTGGTGGATGAGTATCAGGACACCAACACCAGCCAGTATGAGCTGGTGAAACTGCTGGTGGGCAGCC |
| | GCGCGCGCTTTACCGTGGTGGGTGACGATGACCAGTCGATCTACTCCTGGCGCGGTGCACGTCCGCAAAA |
| | CCTGGTGCTGCTGAGTCAGGATTTTCCGGCGCTGAAGGTGATTAAGCTTGAGCAGAACTATCGCTCTTCC |
| | GGGCGTATTCTGAAAGCGGCGAACATCCTGATCGCCAATAACCCGCACGTCTTTGAAAAGCGTCTGTTCT |
| | CCGAACTGGGTTATGGCGCGGAGCTAAAAGTATTAAGCGCGAATAACGAAGAACATGAGGCTGAGCGCGT |
| | TACTGGCGAGCTGATCGCCCATCACTTCGTCAATAAAACGCAGTACAAAGATTACGCCATTCTTTATCGC |
| | GGTAACCATCAGTCGCGGGTGTTTGAAAAATTCCTGATGCAAAACCGCATCCCGTACAAAATATCTGGTG |
| | GTACGTCGTTTTTCTCTCGTCCTGAAATCAAGGACTTGCTGGCTTATCTGCGCGTGCTGACTAACCCGGA |
| | CGATGACAGCGCATTTCTGCGTATCGTTAACACGCCGAAGCGAGAGATTGGCCCGGCTACGCTGAAAAAG |
| | CTGGGTGAGTGGGCGATGACGCGCAATAAAAGCATGTTTACCGCCAGCTTTGATATGGGCCTGAGTCAGA |
| | CGCTTAGCGGACGTGGTTATGAAGCATTGACCCGCTTCACTCACTGGTTGGCAGAAATCCAGCGTCTGGC |
| | GGAGCGGGAGCCGATTGCCGCGGTGCGTGATCTGATCCATGGCATGGATTATGAATCCTGGCTGTACGAA |
| | ACATCGCCCAGCCCGAAAGCCGCCGAAATGCGCATGAAGAACGTCAACCAACTGTTTAGCTGGATGACGG |
| | AGATGCTGGAAGGCAGTGAACTGGATGAGCCGATGACGCTCACCCAGGTGGTGACGCGCTTTACTTTGCG |
| | CGACATGATGGAGCGTGGTGAGAGTGAAGAAGAGCTGGATCAGGTGCAACTGATGACTCTCCACGCGTCG |
| | AAAGGGCTGGAGTTTCCTTATGTCTACATGGTCGGTATGGAAGAAGGGTTTTTGCCGCACCAGAGCAGCA |
| | TCGATGAAGATAATATCGATGAGGAGCGGCGGCTGGCCTATGTCGGCATTACCCGCGCCCAGAAGGAATT |
| | GACCTTTACGCTGTGTAAAGAACGCCGTCAGTACGGCGAACTGGTGCGCCCGGAGCCGAGCCGCTTTTTG |
| | CTGGAGCTGCCGCAGGATGATCTGATTTGGGAACAGGAGCGCAAAGTGGTCAGCGCCGAAGAACGGATGC |
| | AGAAAGGGCAAAGCCATCTGGCGAATCTGAAAGCGATGATGGCGGCAAAACGAGGGAAATAA |

Embodiments of Linker Amino Acid Sequences

| | |
|---|---|
| 6 | >one embodiment of linker or fragment thereof derived from x-synuclein |
| | VGSKTKEGVVHGVATVAEKTKEQVTN |
| 7 | >one embodiment of glycine rich linker or fragment thereof |
| | GGGGSGGGGSGGGGSGGGGS |
| 8 | >one embodiment of glycine rich linker or fragment thereof |
| | GGGASVTGGSAGAGSTVSGAGSIAGSGGGGSGGGG |
| 9 | >one embodiment of linker |
| | GGGGSGGGGSGGGGSGGGGSVGSKTKEGVVHGVATVAEKTKEQVTNGGGASVTGGSAGAGSTVSGAGSIA |
| | GSGGGGSGGGG |

Embodiments of T5-Rep fusion polypeptide

| | |
|---|---|
| 10 | > one embodiment of T5-Rep fusion (D164A, E166A, A593R, and truncated |
| | Rep) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTEVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDIMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLFPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGAGGGGSGGGGSGGGGSGGGGSVGSKTKEGVVHGVATVAEKTKEQVTNGGGASVTGG |
| | SAGAGSTVSGAGSIAGSGGGGSGGGGRLNPGQQQAVEFVTGPCLVLAGAGSGKTRVITNKIAHLIRGCGY |
| | QARHIAAVTFTNKAAREMKERVGQTLGRKEARGLMISTFHTLGLDIIKREYAALGMKANFSLFDDTDQLA |
| | LLKELTFGLIEDDKVLLQQLISTISNWKNDLKTPSQAAASAIGERDRIFAHCYGLYDAHLKACNVLDFDD |
| | LILLPTLLLQRNEEVRKRWQNKIRYLLVDEYQDTNTSQYELVKLLVGSRARFTVVGDDDQSIYSWRGARP |
| | QNLVLLSQDFPALKVIKLEQNYRSSGRILKAANILIANNPHVFEKRLFSELGYGAELKVLSANNEEHEAE |
| | RVTGELIAHHFVNKTQYKDYAILYRGNHQSRVFEKFLMQNRIPYKISGGTSFFSRPEIKDLLAYLRVLTN |
| | PDDDSAFLRIVNTPKREIGPATLKKLGEWAMTRNKSMFTASFDMGLSQTLSGRGYEALTRFTHWLAEIQR |
| | LAEREPIAAVRDLIHGMDYESWLYETSPSPKAAEMRMKNVNQLFSWMTEMLEGSELDEPMTLTQVVTRFT |
| | LRDMMERGESEEELDQVQLMTLHASKGLEFPYVYMVGMEEGFLPHQSSIDEDNIDEERRLAYVGITRAQK |
| | ELTFTLCKERRQYGELVRPEPSRFLLELPQDDLIW |
| 11 | > one embodiment of T5-Rep fusion (D164A, E166A, I308V, A593R, and |
| | truncated Rep) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTFVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDVMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLEPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGAGGGGSGGGGSGGGGSGGGGSVGSKTKEGVVHGVATVAEKTKEQVTNGGGASVTGG |
| | SAGAGSTVSGAGSIAGSGGGGSGGGGRLNPGQQQAVEFVTGPCLVLAGAGSGKTRVITNKIAHLIRGCGY |
| | QARHIAAVTFTNKAAREMKERVGQTLGRKEARGLMISTFHTLGLDIIKREYAALGMKANFSLFDDTDQLA |
| | LLKELTFGLIEDDKVLLQQLISTISNWKNDLKTPSQAAASAIGERDRIFAHCYGLYDAHLKACNVLDFDD |
| | LILLPTLLLQRNEEVRKRWQNKIRYLLVDEYQDTNTSQYELVKLLVGSRARFTVVGDDDQSIYSWRGARP |
| | QNLVLLSQDFPALKVIKLEQNYRSSGRILKAANILIANNPHVFEKRLFSELGYGAELKVLSANNEEHEAE |
| | RVTGELIAHHFVNKTQYKDYAILYRGNHQSRVFEKFLMQNRIPYKISGGTSFFSRPEIKDLLAYLRVLTN |
| | PDDDSAFLRIVNTPKREIGPATLKKLGEWAMTRNKSMFTASFDMGLSQTLSGRGYEALTRFTHWLAEIQR |
| | LAEREPIAAVRDLIHGMDYESWLYETSPSPKAAEMRMKNVNQLFSWMTEMLEGSELDEPMTLTQVVTRFT |
| | LRDMMERGESEEELDQVQLMTLHASKGLEFPYVYMVGMEEGFLPHQSSIDEDNIDEERRLAYVGITRAQK |
| | ELTFTLCKERRQYGELVRPEPSRFLLELPQDDLIW |
| 22 | > one embodiment of T5-Rep fusion (D164A, E166A, A593R) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTFVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDIMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLFPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGFNAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGAGGGGSGGGGSGGGGSGGGGSVGSKTKEGVVHGVATVAEKTKEQVTNGGGASVTGG |
| | SAGAGSTVSGAGSIAGSGGGGSGGGGRLNPGQQQAVEFVTGPCLVLAGAGSGKTRVITNKIAHLIRGCGY |
| | QARHIAAVTFTNKAAREMKERVGQTLGRKEARGLMISTFHTLGLDIIKREYAALGMKANFSLFDDTDQLA |
| | LLKELTFGLIEDDKVLLQQLISTISNWKNDLKTPSQAAASAIGERDRIFAHCYGLYDAHLKACNVLDFDD |
| | LILLPTLLLQRNEEVRKRWQNKIRYLLVDEYQDTNTSQYELVKLLVGSRARFTVVGDDDQSIYSWRGARP |
| | QNLVLLSQDFPALKVIKLEQNYRSSGRILKAANILIANNPHVFEKRLFSELGYGAELKVLSANNEEHEAE |
| | RVTGELIAHHFVNKTQYKDYAILYRGNHQSRVFEKFLMQNRIPYKISGGTSFFSRPEIKDLLAYLRVLTN |
| | PDDDSAFLRIVNTPKREIGPATLKKLGEWAMTRNKSMFTASFDMGLSQTLSGRGYEALTRFTHWLAEIQR |
| | LAEREPIAAVRDLIHGMDYESWLYETSPSPKAAEMRMKNVNQLFSWMTEMLEGSELDEPMTLTQVVTRFT |
| | LRDMMERGESEEELDQVQLMTLHASKGLEFPYVYMVGMEEGFLPHQSSIDEDNIDEERRLAYVGITRAQK |
| | ELTFTLCKERRQYGELVRPEPSRFLLELPQDDLIWEQERKVVSAEERMQKGQSHLANLKAMMAAKRGK |
| 23 | > one embodiment of T5-Rep fusion (D164A, E166A, I308V, A593R) |
| | MKIAVVDKALNNTRYDKHFQLYGEEVDVFHMCNEKLSGRLLKKHITIGTPENPFDPNDYDFVILVGAEPF |
| | LYFAGKKGIGDYTGKRVEYNGYANWIASISPAQLHFKPEMKPVFDATVENIHDIINGREKIAKAGDYRPI |
| | TDPDEAEEYIKMVYNMVIGPVAFASATSALYCRDGYLLGVSISHQEYQGVYIDSDCLTFVAVYYLQKILD |
| | SENHTIVFHNLKFDMHFYKYHLGLTFDKAHKERRLHDTMLQHYVLDERRGTHGLKSLAMKYTDMGDYDFE |
| | LDKFKDDYCKAHKIKKEDFTYDLIPFDVMWPYAAKDTDATIRLHNFFLPKIEKNEKLCSLYYDVLMPGCV |
| | FLQRVEDRGVPISIDRLKEAQYQLTHNLNKAREKLYTYPEVKQLEQDQNEAFNPNSVKQLRVLLFDYVGL |
| | TPTGKLTDTGADSTDAEALNELATQHPIAKTLLEIRKLTKLISTYVEKILLSIDADGCIRTGFHEHMTTS |
| | GRLSSSGKLNLQQLPRDESIIKGCVVAPPGYRVIAWDLTTAEVYYAAVLSGDRNMQQVFINMRNEPDKYP |
| | DFHSNIAHMVFKLQCEPRDVKKLFPALRQAAKRITFGILYGSGPAKVAHSVNEALLEQAAKTGEPFVECT |
| | VADAKEYIETYFGQFPQLKRWIDKCHDQIKNHGFIYSHFGRKRRLHNIHSEDRGVQGEEIRSGENAIIQS |
| | ASSDSLLLGAVDADNEIISLGLEQEMKIVMLVHDSVVAIVREDLIDQYNEILIRNIQKDRGISIPGCPIG |
| | IDSDSEAGGSRDYSCGKMKKQHPSIACIDDDEYTRYVKGVLLDAEFEYKKLAAMDKEHPDHSKYKDDKFI |
| | AVCKDLDNVKRILGAGGGGSGGGGSGGGGSGGGGSVGSKTKEGVVHGVATVAEKTKEQVTNGGGASVTGG |
| | SAGAGSTVSGAGSIAGSGGGGSGGGGRLNPGQQQAVEFVTGPCLVLAGAGSGKTRVITNKIAHLIRGCGY |
| | QARHIAAVTFTNKAAREMKERVGQTLGRKEARGLMISTFHTLGLDIIKREYAALGMKANFSLFDDTDQLA |
| | LLKELTFGLIEDDKVLLQQLISTISNWKNDLKTPSQAAASAIGERDRIFAHCYGLYDAHLKACNVLDFDD |
| | LILLPTLLLQRNEEVRKRWQNKIRYLLVDEYQDTNTSQYELVKLLVGSRARFTVVGDDDQSIYSWRGARP |
| | QNLVLLSQDFPALKVIKLEQNYRSSGRILKAANILIANNPHVFEKRLFSELGYGAELKVLSANNEEHEAE |
| | RVTGELIAHHFVNKTQYKDYAILYRGNHQSRVFEKELMQNRIPYKISGGTSFFSRPEIKDLLAYLRVLTN |
| | PDDDSAFLRIVNTPKREIGPATLKKLGEWAMTRNKSMFTASFDMGLSQTLSGRGYEALTRFTHWLAEIQR |
| | LAEREPIAAVRDLIHGMDYESWLYETSPSPKAAEMRMKNVNQLFSWMTEMLEGSELDEPMTLTQVVTRFT |
| | LRDMMERGESEEELDQVQLMTLHASKGLEFPYVYMVGMEEGFLPHQSSIDEDNIDEERRLAYVGITRAQK |
| | ELTFTLCKERRQYGELVRPEPSRFLLELPQDDLIWEQERKVVSAEERMQKGQSHLANLKAMMAAKRGK |
| | Embodiments of Nucleic Acid Sequences encoding T5-Rep Fusion Protein |
| 12 | > one embodiment of T5-Rep fusion using T5 mutant (D164A, E166A, A593R) |
| | ATGAAAATCGCAGTAGTTGATAAAGCTCTAAACAACACTCGTTATGATAAACATTTCCAGCTATACGGCG |
| | AGGAAGTTGATGTATTCCATATGTGTAACGAGAAGTTGTCCGGTCGTTTGCTCAAAAAGCATATTACTAT |
| | CGGAACTCCGGAAAACCCATTTGACCCGAATGATTATGATTTTGTTATACTGGTAGGTGCCGAACCTTTC |
| | CTGTACTTTGCAGGTAAGAAAGGTATTGGTGATTATACCGGTAAACGTGTAGAGTATAATGGATATGCTA |
| | ACTGGATTGCGAGTATCAGCCCAGCCCAGTTACACTTTAAACCTGAAATGAAACCAGTTTTTGATGCAAC |
| | AGTAGAGAATATCCACGATATTATCAATGGTCGTGAGAAGATTGCAAAAGCTGGTGATTACCGTCCTATT |
| | ACTGACCCTGATGAGGCTGAAGAATATATCAAGATGGTGTATAATATGGTTATCGGACCCGTCGCATTCG |
| | CCTCCGCAACCTCAGCACTATACTGTCGAGATGGTTATCTGCTTGGTGTTTCTATTTCTCACCAAGAGTA |
| | TCAGGGTGTATATATCGATTCTGATTGTCTCACAGAGGTTGCAGTATATTATCTCCAGAAAATTCTGGAT |
| | AGTGAAAACCACACTATTGTTTTTCACAACTTGAAGTTTGATATGCACTTTTATAAGTACCATCTGGGAC |
| | TTACTTTTGATAAAGCACATAAAGAACGCAGGCTCCATGATACCATGTTGCAGCACTATGTTTTAGATGA |
| | ACGTCGTGGTACTCATGGCTTGAAATCTCTAGCAATGAAGTATACCGATATGGGTGACTATGACTTCGAA |
| | CTAGATAAGTTCAAAGATGATTACTGTAAAGCACATAAAATCAAGAAAGAAGATTTCACCTATGATTTAA |
| | TTCCGTTTGATGTTATGTGGCCATATGCTGCGAAAGATACGGATGCCACTATACGTTTGCACAACTTCTT |
| | TTTACCAAAAATTGAGAAGAATGAAAAACTTTGCAGTCTGTATTACGATGTTTTGATGCCTGGTTGCGTA |
| | TTCTTGCAACGTGTTGAGGATCGTGGAGTACCTATCTCTATTGATCGTTTGAAAGAAGCTCAGTATCAGT |
| | TGACTCATAATTTGAATAAAGCCCGTGAGAAACTGTACACTTATCCAGAAGTTAAACAGCTAGAACAAGA |
| | TCAGAATGAAGCATTTAACCCGAACTCTGTTAAGCAGCTACGTGTTCTTCTGTTTGATTACGTTGGCTTA |
| | ACTCCAACAGGTAAACTGACGGATACTGGAGCAGATTCTACGGATGCAGAAGCTCTAAATGAACTGGCTA |
| | CGCAGCATCCAATTGCTAAAACTCTGCTAGAGATTCGTAAGCTGACTAAGCTGATCTCTACTTATGTTGA |
| | GAAGATTCTACTGAGTATTGATGCAGATGGTTGCATTCGTACTGGTTTCCATGAACATATGACTACTTCT |
| | GGTCGTCTGAGTTCTTCTGGTAAACTGAACCTGCAACAGTTACCCCGTGATGAATCTATTATCAAGGGTT |
| | GTGTAGTAGCTCCTCCTGGGTATCGTGTAATCGCGTGGGACTTAACAACTGCGGAAGTTTATTATGCTGC |
| | TGTTCTATCTGGTGATAGAAATATGCAACAGGTATTTATCAACATGAGAAATGAACCCGATAAATACCCA |
| | GACTTCCACTCCAACATCGCACACATGGTGTTTAAGCTGCAATGCGAACCCCGTGATGTTAAAAAGCTGT |
| | TCCCAGCTCTGCGTCAGGCTGCTAAACGCATCACCTTCGGTATTCTGTATGGTTCTGGCCCAGCTAAAGT |
| | AGCGCATTCTGTTAACGAAGCATTACTAGAACAAGCAGCCAAGACGGGCGAACCGTTTGTTGAATGTACT |
| | GTTGCAGATGCTAAAGAGTACATTGAGACTTACTTCGGTCAGTTCCCACAGCTTAAGCGTTGGATTGATA |
| | AGTGCCACGATCAGATCAAGAATCATGGATTTATCTATAGTCACTTTGGTCGTAAACGTCGTCTGCATAA |
| | TATCCATTCCGAAGACCGTGGTGTTCAGGGTGAAGAAATTCGTTCTGGATTTAATGCAATCATTCAGTCT |
| | GCTTCTTCTGATAGTCTCCTTTTAGGTGCTGTAGATGCAGATAATGAGATCATTTCTCTTGGTTTAGAAC |
| | AAGAGATGAAGATTGTTATGTTGGTTCATGACTCCGTAGTTGCTATTGTTCGTGAGGATTTGATCGACCA |
| | ATACAATGAAATCCTGATTCGTAATATTCAGAAAGACCGTGGTATCAGTATTCCTGGCTGTCCGATTGGT |
| | ATTGATTCAGATTCTGAAGCTGGAGGTTCTCGTGACTATTCTTGTGGTAAGATGAAGAAACAGCACCCAT |
| | CAATCGCTTGTATTGATGATGATGAATATACTCGTTATGTCAAGGGTGTATTACTTGATGCAGAATTCGA |
| | GTATAAGAAACTAGCTGCAATGGATAAAGAGCATCCAGATCATAGCAAATACAAGGATGATAAGTTTATT |
| | GCTGTATGTAAAGATTTGGATAACGTGAAAAGGATTCTCGGTGCTGGAGGCGGTGGGTCTGGTGGTGGAG |
| | GCTCCGGTGGCGGAGGATCAGGGGGAGGTGGTTCGGTGGGCAGTAAGACGAAAGAAGGGGTGGTGCATGG |
| | TGTCGCAACTGTCGCCGAAAAAACAAAGGAACAAGTTACTAACGGAGGCGGCGCGTCAGTTACTGGTGGG |
| | AGCGCTGGTGCTGGATCTACAGTATCGGGCGCGGGTAGTATTGCTGGCTCTGGTGGGGGAGGTTCGGGCG |
| | GCGGTGGCCGTCTAAACCCCGGCCAACAACAAGCTGTCGAATTCGTTACCGGCCCCTGCCTGGTGCTGGC |
| | GGGCGCGGGTTCCGGTAAAACTCGTGTTATCACCAATAAAATCGCCCATCTGATCCGCGGTTGCGGTTAT |
| | CAGGCGCGGCACATTGCGGCGGTGACCTTTACTAATAAAGCAGCGCGCGAGATGAAAGAGCGTGTAGGGC |
| | AGACGCTGGGGCGCAAAGAGGCGCGTGGGCTGATGATCTCCACTTTCCATACGTTGGGGCTGGATATCAT |
| | CAAACGCGAGTATGCGGCGCTTGGGATGAAAGCGAACTTCTCGTTGTTTGACGATACCGATCAGCTTGCT |
| | TTGCTTAAAGAGTTGACCGAGGGGCTGATTGAAGATGACAAAGTTCTCCTGCAACAACTGATTTCGACCA |
| | TCTCTAACTGGAAGAATGATCTCAAAACACCGTCCCAGGCGGCAGCAAGTGCGATTGGCGAGCGGGACCG |
| | TATTTTTGCCCATTGTTATGGGCTGTATGATGCACACCTGAAAGCCTGTAACGTTCTCGACTTCGATGAT |
| | CTGATTTTATTGCCGACGTTGCTGCTGCAACGCAATGAAGAAGTCCGCAAGCGCTGGCAGAACAAAATTC |
| | GCTATCTGCTGGTGGATGAGTATCAGGACACCAACACCAGCCAGTATGAGCTGGTGAAACTGCTGGTGGG |
| | CAGCCGCGCGCGCTTTACCGTGGTGGGTGACGATGACCAGTCGATCTACTCCTGGCGCGGTGCACGTCCG |
| | CAAAACCTGGTGCTGCTGAGTCAGGATTTTCCGGCGCTGAAGGTGATTAAGCTTGAGCAGAACTATCGCT |
| | CTTCCGGGCGTATTCTGAAAGCGGCGAACATCCTGATCGCCAATAACCCGCACGTCTTTGAAAAGCGTCT |
| | GTTCTCCGAACTGGGTTATGGCGCGGAGCTAAAAGTATTAAGCGCGAATAACGAAGAACATGAGGCTGAG |
| | CGCGTTACTGGCGAGCTGATCGCCCATCACTTCGTCAATAAAACGCAGTACAAAGATTACGCCATTCTTT |
| | ATCGCGGTAACCATCAGTCGCGGGTGTTTGAAAAATTCCTGATGCAAAACCGCATCCCGTACAAAATATC |
| | TGGTGGTACGTCGTTTTTCTCTCGTCCTGAAATCAAGGACTTGCTGGCTTATCTGCGCGTGCTGACTAAC |
| | CCGGACGATGACAGCGCATTTCTGCGTATCGTTAACACGCCGAAGCGAGAGATTGGCCCGGCTACGCTGA |
| | AAAAGCTGGGTGAGTGGGCGATGACGCGCAATAAAAGCATGTTTACCGCCAGCTTTGATATGGGCCTGAG |
| | TCAGACGCTTAGCGGACGTGGTTATGAAGCATTGACCCGCTTCACTCACTGGTTGGCAGAAATCCAGCGT |
| | CTGGCGGAGCGGGAGCCGATTGCCGCGGTGCGTGATCTGATCCATGGCATGGATTATGAATCCTGGCTGT |
| | ACGAAACATCGCCCAGCCCGAAAGCCGCCGAAATGCGCATGAAGAACGTCAACCAACTGTTTAGCTGGAT |
| | GACGGAGATGCTGGAAGGCAGTGAACTGGATGAGCCGATGACGCTCACCCAGGTGGTGACGCGCTTTACT |
| | TTGCGCGACATGATGGAGCGTGGTGAGAGTGAAGAAGAGCTGGATCAGGTGCAACTGATGACTCTCCACG |
| | CGTCGAAAGGGCTGGAGTTTCCTTATGTCTACATGGTCGGTATGGAAGAAGGGTTTTTGCCGCACCAGAG |
| | CAGCATCGATGAAGATAATATCGATGAGGAGCGGCGGCTGGCCTATGTCGGCATTACCCGCGCCCAGAAG |
| | GAATTGACCTTTACGCTGTGTAAAGAACGCCGTCAGTACGGCGAACTGGTGCGCCCGGAGCCGAGCCGCT |
| | TTTTGCTGGAGCTGCCGCAGGATGATCTGATTTGGTAA |

Embodiments of CisA Sequences

| | |
|---|---|
| 24 | >sp\|P03631. 1\|REPA_BPPHS, Replication-associated protein A |
| | MVRSYYPSECHADYFDFERIEALKPAIEACGISTLSQSPMLGFHKQMDNRIKLLEEILSFRMQGVEFDNG |
| | DMYVDGHKAASDVRDEFVSVTEKLMDELAQCYNVLPQLDINNTIDHRPEGDEKWFLENEKTVTQFCRKLA |
| | AERPLKDIRDEYNYPKKKGIKDECSRLLEASTMKSRRGFAIQRLMNAMRQAHADGWFIVFDTLTLADDRL |
| | EAFYDNPNALRDYFRDIGRMVLAAEGRKANDSHADCYQYFCVPEYGTANGRLHFHAVHFMRTLPTGSVDP |
| | NFGRRVRNRRQLNSLQNTWPYGYSMPIAVRYTQDAFSRSGWLWPVDAKGEPLKATSYMAVGFYVAKYVNK |
| | KSDMDLAAKGLGAKEWNNSLKTKLSLLPKKLFRIRMSRNFGMKMLTMTNLSTECLIQLTKLGYDATPFNQ |
| | ILKQNAKREMRLRLGKVTVADVLAAQPVTTNLLKFMRASIKMIGVSNLQSFIASMTQKLTLSDISDESKN |
| | YLDKAGITTACLRIKSKWTAGGK |
| 30 | > one embodiment of DNA nickase, CisA mutant with Y303H (bold) |
| | MVRSYYPSECHADYFDFERIEALKPAIEACGISTLSQSPMLGFHKQMDNRIKLLEEILSFRMQGVEFDNG |
| | DMYVDGHKAASDVRDEFVSVTEKLMDELAQCYNVLPQLDINNTIDHRPEGDEKWFLENEKTVTQFCRKLA |
| | AERPLKDIRDEYNYPKKKGIKDECSRLLEASTMKSRRGFAIQRLMNAMRQAHADGWFIVFDTLTLADDRL |
| | EAFYDNPNALRDYFRDIGRMVLAAEGRKANDSHADCYQYFCVPEYGTANGRLHFHAVHFMRTLPTGSVDP |
| | NFGRRVRNRRQLNSLQNTWPYGHSMPIAVRYTQDAFSRSGWLWPVDAKGEPLKATSYMAVGFYVAKYVNK |
| | KSDMDLAAKGLGAKEWNNSLKTKLSLLPKKLFRIRMSRNFGMKMLTMTNLSTECLIQLTKLGYDATPFNQ |
| | ILKQNAKREMRLRLGKVTVADVLAAQPVTTNLLKFMRASIKMIGVSNLQSFIASMTQKLTLSDISDESKN |
| | YLDKAGITTACLRIKSKWTAGGK |
| 25 | >nucleic acid encoding DNA nickase CisA protein with Y303H (SEQ ID |
| | NO: 30) |
| | ATGGTTCGTTCTTATTACCCTTCTGAATGTCACGCTGATTATTTTGACTTTGAGCGTATCGAGGCTCTTA |
| | AACCTGCTATTGAGGCTTGTGGCATTTCTACTCTTTCTCAATCCCCAATGCTTGGCTTCCATAAGCAGAT |
| | GGATAACCGCATCAAGCTCTTGGAAGAGATTCTGTCTTTTCGTATGCAGGGCGTTGAGTTCGATAATGGT |
| | GATATGTATGTTGACGGCCATAAGGCTGCTTCTGACGTTCGTGATGAGTTTGTATCTGTTACTGAGAAGT |
| | TAATGGATGAATTGGCACAATGCTACAATGTGCTCCCCCAGCTGGACATTAACAATACCATTGATCATCG |
| | CCCCGAAGGGGACGAAAAATGGTTTTTAGAGAACGAGAAGACGGTTACGCAGTTTTGCCGCAAGCTGGCT |
| | GCTGAACGCCCTCTTAAGGATATTCGCGATGAGTATAATTACCCCAAAAAGAAAGGTATTAAGGATGAGT |
| | GTTCAAGATTGCTTGAAGCCTCCACTATGAAATCGCGTAGAGGCTTTGCTATTCAGCGTTTGATGAATGC |
| | AATGCGACAGGCTCATGCTGATGGTTGGTTTATCGTTTTTGACACTCTCACGTTGGCTGACGACCGATTA |
| | GAGGCGTTTTATGATAATCCCAATGCTTTGCGTGACTATTTTCGTGATATTGGTCGTATGGTTCTTGCTG |
| | CCGAGGGTCGCAAGGCTAATGATTCACACGCCGACTGCTATCAGTATTTTTGTGTGCCTGAGTATGGTAC |
| | AGCTAATGGCCGTCTTCATTTCCATGCGGTGCACTTTATGCGGACACTTCCTACAGGTAGCGTTGACCCT |
| | AATTTTGGTCGTCGGGTACGCAATCGCCGCCAGTTAAATAGCTTGCAAAATACGTGGCCTTATGGTCACA |
| | GTATGCCCATCGCAGTTCGCTACACGCAGGACGCTTTTTCACGTTCTGGTTGGTTGTGGCCTGTTGATGC |
| | TAAAGGTGAGCCGCTTAAAGCTACCAGTTATATGGCTGTTGGTTTCTATGTGGCTAAATACGTTAACAAA |
| | AAGTCAGATATGGACCTTGCTGCTAAAGGTCTAGGAGCTAAAGAATGGAACAACTCACTAAAAACCAAGC |
| | TGTCGCTACTTCCCAAGAAGCTGTTCAGAATCAGAATGAGCCGCAACTTCGGGATGAAAATGCTCACAAT |
| | GACAAATCTGTCCACGGAGTGCTTAATCCAACTTACCAAGCTGGGTTACGACGCGACGCCGTTCAACCAG |
| | ATATTGAAGCAGAACGCAAAAAGAGAGATGAGATTGAGGCTGGGAAAAGTTACTGTAGCCGACGTTTTGG |
| | CGGCGCAACCTGTGACGACAAATCTGCTCAAATTTATGCGCGCTTCGATAAAAATGATTGGCGTATCCAA |
| | CCTGCAGAGTTTTATCGCTTCCATGACGCAGAAGTTAACACTTTCGGATATTTCTGATGAGTCGAAAAAT |
| | TATCTTGATAAAGCAGGAATTACTACTGCTTGTTTACGAATTAAATCGAAGTGGACTGCTGGCGGAAAAT |
| | GA |

Embodiments of CisA Initiation Sequences

| | |
|---|---|
| 13 | > one embodiment of CisA initiation sequence |
| | CAACTTGATATTAATAACACTATAGACCAC |

Embodiments of CisA termination Sequences

| | |
|---|---|
| 14 | > one embodiment of CisA termination sequence |
| | CAACTTGATATTAATAACACTATAACTTCT |
| 21 | > one embodiment of CisA termination sequence |
| | CAACTTGATATTAATAACACTATA |

Additional Exemplary Nucleic Acid Sequences

| | |
|---|---|
| 15 | > T7 promoter sequence |
| | TAATACGACTCACTATAG |
| 16 | > one embodiment of modified 5′UTR (ribosomal binding site is bolded) |
| | GCTCTATCCAGAAGTTATAGTGTTATTAATATTAGGTTGAGGTTATAA |
| 17 | > T7 terminator sequence |
| | CTAGCATAACCCCTTGGGGCCTCTAAACGGGTCTTGAGGGGTTTTTTG |
| 18 | > one embodiment of helper vector plasmid with RepAC33, I308V and 5′ |
| | UTR modified (T7 promoter underlined, T7 terminator bold underlined, T5 |
| | gene bolded, RepΔC33 helicase italicized) |
| | TAATACGACTCACTATAGGCTCTATCCAGAAGTTATAGTGTTATTAATATTAGGTTGAGGTTATAAATGA |
| | AAATCGCAGTAGTTGATAAAGCTCTAAACAACACTCGTTATGATAAACATTTCCAGCTATACGGCGAGGA |
| | AGTTGATGTATTCCATATGTGTAACGAGAAGTTGTCCGGTCGTTTGCTCAAAAAGCATATTACTATCGGA |
| | ACTCCGGAAAACCCATTTGACCCGAATGATTATGATTTTGTTATACTGGTAGGTGCCGAACCTTTCCTGT |
| | ACTTTGCAGGTAAGAAAGGTATTGGTGATTATACCGGTAAACGTGTAGAGTATAATGGATATGCTAACTG |
| | GATTGCGAGTATCAGCCCAGCCCAGTTACACTTTAAACCTGAAATGAAACCAGTTTTTGATGCAACAGTA |
| | GAGAATATCCACGATATTATCAATGGTCGTGAGAAGATTGCAAAAGCTGGTGATTACCGTCCTATTACTG |
| | ACCCTGATGAGGCTGAAGAATATATCAAGATGGTGTATAATATGGTTATCGGACCCGTCGCATTCGCCTC |
| | CGCAACCTCAGCACTATACTGTCGAGATGGTTATCTGCTTGGTGTTTCTATTTCTCACCAAGAGTATCAG |
| | GGTGTATATATCGATTCTGATTGTCTCACAGAGGTTGCAGTATATTATCTCCAGAAAATTCTGGATAGTG |
| | AAAACCACACTATTGTTTTTCACAACTTGAAGTTTGATATGCACTTTTATAAGTACCATCTGGGACTTAC |
| | TTTTGATAAAGCACATAAAGAACGCAGGCTCCATGATACCATGTTGCAGCACTATGTTTTAGATGAACGT |
| | CGTGGTACTCATGGCTTGAAATCTCTAGCAATGAAGTATACCGATATGGGTGACTATGACTTCGAACTAG |
| | ATAAGTTCAAAGATGATTACTGTAAAGCACATAAAATCAAGAAAGAAGATTTCACCTATGATTTAATTCC |
| | GTTTGATGTTATGTGGCCATATGCTGCGAAAGATACGGATGCCACTATACGTTTGCACAACTTCTTTTTA |
| | CCAAAAATTGAGAAGAATGAAAAACTTTGCAGTCTGTATTACGATGTTTTGATGCCTGGTTGCGTATTCT |
| | TGCAACGTGTTGAGGATCGTGGAGTACCTATCTCTATTGATCGTTTGAAAGAAGCTCAGTATCAGTTGAC |
| | TCATAATTTGAATAAAGCCCGTGAGAAACTGTACACTTATCCAGAAGTTAAACAGCTAGAACAAGATCAG |
| | AATGAAGCATTTAACCCGAACTCTGTTAAGCAGCTACGTGTTCTTCTGTTTGATTACGTTGGCTTAACTC |
| | CAACAGGTAAACTGACGGATACTGGAGCAGATTCTACGGATGCAGAAGCTCTAAATGAACTGGCTACGCA |
| | GCATCCAATTGCTAAAACTCTGCTAGAGATTCGTAAGCTGACTAAGCTGATCTCTACTTATGTTGAGAAG |
| | ATTCTACTGAGTATTGATGCAGATGGTTGCATTCGTACTGGTTTCCATGAACATATGACTACTTCTGGTC |
| | GTCTGAGTTCTTCTGGTAAACTGAACCTGCAACAGTTACCCCGTGATGAATCTATTATCAAGGGTTGTGT |
| | AGTAGCTCCTCCTGGGTATCGTGTAATCGCGTGGGACTTAACAACTGCGGAAGTTTATTATGCTGCTGTT |
| | CTATCTGGTGATAGAAATATGCAACAGGTATTTATCAACATGAGAAATGAACCCGATAAATACCCAGACT |
| | TCCACTCCAACATCGCACACATGGTGTTTAAGCTGCAATGCGAACCCCGTGATGTTAAAAAGCTGTTCCC |
| | AGCTCTGCGTCAGGCTGCTAAACGCATCACCTTCGGTATTCTGTATGGTTCTGGCCCAGCTAAAGTAGCG |
| | CATTCTGTTAACGAAGCATTACTAGAACAAGCAGCCAAGACGGGCGAACCGTTTGTTGAATGTACTGTTG |
| | CAGATGCTAAAGAGTACATTGAGACTTACTTCGGTCAGTTCCCACAGCTTAAGCGTTGGATTGATAAGTG |
| | CCACGATCAGATCAAGAATCATGGATTTATCTATAGTCACTTTGGTCGTAAACGTCGTCTGCATAATATC |
| | CATTCCGAAGACCGTGGTGTTCAGGGTGAAGAAATTCGTTCTGGATTTAATGCAATCATTCAGTCTGCTT |
| | CTTCTGATAGTCTCCTTTTAGGTGCTGTAGATGCAGATAATGAGATCATTTCTCTTGGTTTAGAACAAGA |
| | GATGAAGATTGTTATGTTGGTTCATGACTCCGTAGTTGCTATTGTTCGTGAGGATTTGATCGACCAATAC |
| | AATGAAATCCTGATTCGTAATATTCAGAAAGACCGTGGTATCAGTATTCCTGGCTGTCCGATTGGTATTG |
| | ATTCAGATTCTGAAGCTGGAGGTTCTCGTGACTATTCTTGTGGTAAGATGAAGAAACAGCACCCATCAAT |
| | CGCTTGTATTGATGATGATGAATATACTCGTTATGTCAAGGGTGTATTACTTGATGCAGAATTCGAGTAT |
| | AAGAAACTAGCTGCAATGGATAAAGAGCATCCAGATCATAGCAAATACAAGGATGATAAGTTTATTGCTG |
| | TATGTAAAGATTTGGATAACGTGAAAAGGATTCTCGGTGCTGGAGGCGGTGGGTCTGGTGGTGGAGGCTC |
| | CGGTGGCGGAGGATCAGGGGGAGGTGGTTCGGTGGGCAGTAAGACGAAAGAAGGGGTGGTGCATGGTGTC |
| | GCAACTGTCGCCGAAAAAACAAAGGAACAAGTTACTAACGGAGGCGGCGCGTCAGTTACTGGTGGGAGCG |
| | CTGGTGCTGGATCTACAGTATCGGGCGCGGGTAGTATTGCTGGCTCTGGTGGGGGAGGTTCGGGCGGCGG |
| | TGGCCGTCTAAACCCCGGCCAACAACAAGCTGTCGAATTCGTTACCGGCCCCTGCCTGGTGCTGGCGGGC |
| | GCGGGTTCCGGTAAAACTCGTGTTATCACCAATAAAATCGCCCATCTGATCCGCGGTTGCGGTTATCAGG |
| | CGCGGCACATTGCGGCGGTGACCTTTACTAATAAAGCAGCGCGCGAGATGAAAGAGCGTGTAGGGCAGAC |
| | GCTGGGGCGCAAAGAGGCGCGTGGGCTGATGATCTCCACTTTCCATACGTTGGGGCTGGATATCATCAAA |
| | CGCGAGTATGCGGCGCTTGGGATGAAAGCGAACTTCTCGTTGTTTGACGATACCGATCAGCTTGCTTTGC |
| | TTAAAGAGTTGACCGAGGGGCTGATTGAAGATGACAAAGTTCTCCTGCAACAACTGATTTCGACCATCTC |
| | TAACTGGAAGAATGATCTCAAAACACCGTCCCAGGCGGCAGCAAGTGCGATTGGCGAGCGGGACCGTATT |
| | TTTGCCCATTGTTATGGGCTGTATGATGCACACCTGAAAGCCTGTAACGTTCTCGACTTCGATGATCTGA |
| | TTTTATTGCCGACGTTGCTGCTGCAACGCAATGAAGAAGTCCGCAAGCGCTGGCAGAACAAAATTCGCTA |
| | TCTGCTGGTGGATGAGTATCAGGACACCAACACCAGCCAGTATGAGCTGGTGAAACTGCTGGTGGGCAGC |
| | CGCGCGCGCTTTACCGTGGTGGGTGACGATGACCAGTCGATCTACTCCTGGCGCGGTGCACGTCCGCAAA |
| | ACCTGGTGCTGCTGAGTCAGGATTTTCCGGCGCTGAAGGTGATTAAGCTTGAGCAGAACTATCGCTCTTC |
| | CGGGCGTATTCTGAAAGCGGCGAACATCCTGATCGCCAATAACCCGCACGTCTTTGAAAAGCGTCTGTTC |
| | TCCGAACTGGGTTATGGCGCGGAGCTAAAAGTATTAAGCGCGAATAACGAAGAACATGAGGCTGAGCGCG |
| | TTACTGGCGAGCTGATCGCCCATCACTTCGTCAATAAAACGCAGTACAAAGATTACGCCATTCTTTATCG |
| | CGGTAACCATCAGTCGCGGGTGTTTGAAAAATTCCTGATGCAAAACCGCATCCCGTACAAAATATCTGGT |
| | GGTACGTCGTTTTTCTCTCGTCCTGAAATCAAGGACTTGCTGGCTTATCTGCGCGTGCTGACTAACCCGG |
| | ACGATGACAGCGCATTTCTGCGTATCGTTAACACGCCGAAGCGAGAGATTGGCCCGGCTACGCTGAAAAA |
| | GCTGGGTGAGTGGGCGATGACGCGCAATAAAAGCATGTTTACCGCCAGCTTTGATATGGGCCTGAGTCAG |
| | ACGCTTAGCGGACGTGGTTATGAAGCATTGACCCGCTTCACTCACTGGTTGGCAGAAATCCAGCGTCTGG |
| | CGGAGCGGGAGCCGATTGCCGCGGTGCGTGATCTGATCCATGGCATGGATTATGAATCCTGGCTGTACGA |
| | AACATCGCCCAGCCCGAAAGCCGCCGAAATGCGCATGAAGAACGTCAACCAACTGTTTAGCTGGATGACG |
| | GAGATGCTGGAAGGCAGTGAACTGGATGAGCCGATGACGCTCACCCAGGTGGTGACGCGCTTTACTTTGC |
| | GCGACATGATGGAGCGTGGTGAGAGTGAAGAAGAGCTGGATCAGGTGCAACTGATGACTCTCCACGCGTC |
| | GAAAGGGCTGGAGTTTCCTTATGTCTACATGGTCGGTATGGAAGAAGGGTTTTTGCCGCACCAGAGCAGC |
| | ATCGATGAAGATAATATCGATGAGGAGCGGCGGCTGGCCTATGTCGGCATTACCCGCGCCCAGAAGGAAT |
| | TGACCTTTACGCTGTGTAAAGAACGCCGTCAGTACGGCGAACTGGTGCGCCCGGAGCCGAGCCGCTTTTT |
| | GCTGGAGCTGCCGCAGGATGATCTGATTTGGTAACTAGCATAACCCCTTGGGGCCTCTAAACGGGTCTTG |
| | AGGGGTTTTTTGTCCATGGGTATGGACAGTTTTCCCTTTGATATGTAACGGTGAACAGTTGTTCTACTTT |
| | TGTTTGTTAGTCTTGATGCTTCACTGATAGATACAAGAGCCATAAGAACCTCAGATCCTTCCGTATTTAG |
| | CCAGTATGTTCTCTAGTGTGGTTCGTTGTTTTTGCGTGAGCCATGAGAACGAACCATTGAGATCATACTT |
| | ACTTTGCATGTCACTCAAAAATTTTGCCTCAAAACTGGTGAGCTGAATTTTTGCAGTTAAAGCATCGTGT |
| | AGTGTTTTTCTTAGTCCGTTACGTAGGTAGGAATCTGATGTAATGGTTGTTGGTATTTTGTCACCATTCA |
| | TTTTTATCTGGTTGTTCTCAAGTTCGGTTACGAGATCCATTTGTCTATCTAGTTCAACTTGGAAAATCAA |
| | CGTATCAGTCGGGCGGCCTCGCTTATCAACCACCAATTTCATATTGCTGTAAGTGTTTAAATCTTTACTT |
| | ATTGGTTTCAAAACCCATTGGTTAAGCCTTTTAAACTCATGGTAGTTATTTTCAAGCATTAACATGAACT |
| | TAAATTCATCAAGGCTAATCTCTATATTTGCCTTGTGAGTTTTCTTTTGTGTTAGTTCTTTTAATAACCA |
| | CTCATAAATCCTCATAGAGTATTTGTTTTCAAAAGACTTAACATGTTCCAGATTATATTTTATGAATTTT |
| | TTTAACTGGAAAAGATAAGGCAATATCTCTTCACTAAAAACTAATTCTAATTTTTCGCTTGAGAACTTGG |
| | CATAGTTTGTCCACTGGAAAATCTCAAAGCCTTTAACCAAAGGATTCCTGATTTCCACAGTTCTCGTCAT |
| | CAGCTCTCTGGTTGCTTTAGCTAATACACCATAAGCATTTTCCCTACTGATGTTCATCATCTGAGCGTAT |
| | TGGTTATAAGTGAACGATACCGTCCGTTCTTTCCTTGTAGGGTTTTCAATCGTGGGGTTGAGTAGTGCCA |
| | CACAGCATAAAATTAGCTTGGTTTCATGCTCCGTTAAGTCATAGCGACTAATCGCTAGTTCATTTGCTTT |
| | GAAAACAACTAATTCAGACATACATCTCAATTGGTCTAGGTGATTTTAATCACTATACCAATTGAGATGG |
| | GCTAGTCAATGATAATTACTAGTCCTTTTCCTTTGAGTTGTGGGTATCTGTAAATTCTGCTAGACCTTTG |
| | CTGGAAAACTTGTAAATTCTGCTAGACCCTCTGTAAATTCCGCTAGACCTTTGTGTGTTTTTTTTGTTTA |
| | TATTCAAGTGGTTATAATTTATAGAATAAAGAAAGAATAAAAAAAGATAAAAAGAATAGATCCCAGCCCT |
| | GTGTATAACTCACTACTTTAGTCAGTTCCGCAGTATTACAAAAGGATGTCGCAAACGCTGTTTGCTCCTC |
| | TACAAAACAGACCTTAAAACCCTAAAGGCTTAAGTAGCACCCTCGCAAGCTCGGTTGCGGCCGCAATCGG |
| | GCAAATCGCTGAATATTCCTTTTGTCTCCGACCATCAGGCACCTGAGTCGCTGTCTTTTTCGTGACATTC |
| | AGTTCGCTGCGCTCACGGCTCTGGCAGTGAATGGGGGTAAATGGCACTACAGGCGCCTTTTATGGATTCA |
| | TGCAAGGAAACTACCCATAATACAAGAAAAGCCCGTCACGGGCTTCTCAGGGCGTTTTATGGCGGGTCTG |
| | CTATGTGGTGCTATCTGACTTTTTGCTGTTCAGCAGTTCCTGCCCTCTGATTTTCCAGTCTGACCACTTC |
| | GGATTATCCCGTGACAGGTCATTCAGACTGGCTAATGCACCCAGTAAGGCAGCGGTATCATCAACGGGGT |
| | CTGACGCTCAGTGGAACGAAAACTCACGTTAAGGGATTTTGGTCATGAGATTATCAAAAAGGATCTTCAC |
| | CTAGATCCTTTTAAATTAAAAATGAAGTTTTAAATCAATCTAAAGTATATATGAGTAAACTTGGTCTGAC |
| | AGTTACCAATGCTTAATCAGTGAGGCACCTATCTCAGCGATCTGTCTATTTCGTTCATCCATAGTTGCCT |
| | GACTCCCCGTCGTGTAGATAACTACGATACGGGAGGGCTTACCATCTGGCCCCAGTGCTGCAATGATACC |
| | GCGAGACCCACGCTCACCGGCTCCAGATTTATCAGCAATAAACCAGCCAGCCGGAAGGGCCGAGCGCAGA |
| | AGTGGTCCTGCAACTTTATCCGCCTCCATCCAGTCTATTAATTGTTGCCGGGAAGCTAGAGTAAGTAGTT |
| | CGCCAGTTAATAGTTTGCGCAACGTTGTTGCCATTGCTACAGGCATCGTGGTGTCACGCTCGTCGTTTGG |
| | TATGGCTTCATTCAGCTCCGGTTCCCAACGATCAAGGCGAGTTACATGATCCCCCATGTTGTGCAAAAAA |
| | GCGGTTAGCTCCTTCGGTCCTCCGATCGTTGTCAGAAGTAAGTTGGCCGCAGTGTTATCACTCATGGTTA |
| | TGGCAGCACTGCATAATTCTCTTACTGTCATGCCATCCGTAAGATGCTTTTCTGTGACTGGTGAGTACTC |
| | AACCAAGTCATTCTGAGAATAGTGTATGCGGCGACCGAGTTGCTCTTGCCCGGCGTCAATACGGGATAAT |
| | ACCGCGCCACATAGCAGAACTTTAAAAGTGCTCATCATTGGAAAACGTTCTTCGGGGCGAAAACTCTCAA |
| | GGATCTTACCGCTGTTGAGATCCAGTTCGATGTAACCCACTCGTGCACCCAACTGATCTTCAGCATCTTT |
| | TACTTTCACCAGCGTTTCTGGGTGAGCAAAAACAGGAAGGCAAAATGCCGCAAAAAAGGGAATAAGGGCG |
| | ACACGGAAATGTTGAATACTCATACTCTTCCTTTTTCAATATTATTGAAGCATTTATCAGGGTTATTGTC |
| | TCATGAGCGGATACATATTTGAATGTATTTAGAAAAATAAACAAATAGGGGTTCCGCGCACATTTCCCCG |
| | AAAAGTGCCACCTGCATCGATTTA |
| 19 | > one embodiment of target vector plasmid with KanR* (underlined) and |
| | GFP (italicized) in target region between the initiation sequence |
| | (bold, italicized) and termination sequence (bold) |
| | TTAATTAACCTAGGCTGCTGCCACCGCTGAGCAATAACTAGCATAACCCCTTGGGGCCTCTAAACGGGTC |
| | TTGAGGGGTTTTTTGCTGAAACCTCAGGCATTTGAGAAGCACACGGTCACACTGCTTCCGGTAGTCAATA |
| | AACCGGTAAACCAGCAATAGACATAAGCGGCTATTTAACGACCCTGCCCTGAACCGACGACCGGGTCGAA |
| | TTTGCTTTCGAATTTCTGCCATTCATCCGCTTATTATCACTTATTCAGGCGTAGCACCAGGCGTTTAAGG |
| | GCACCAATAACTGCCTTAAAAAAATTACGCCCCGCCCTGCCACTCATCGCAGTACTGTTGTAATTCATTA |
| | AGCATTCTGCCGACATGGAAGCCATCACAGACGGCATGATGAACCTGAATCGCCAGCGGCATCAGCACCT |
| | TGTCGCCTTGCGTATAATATTTGCCCATAGTGAAAACGGGGGCGAAGAAGTTGTCCATATTGGCCACGTT |
| | TAAATCAAAACTGGTGAAACTCACCCAGGGATTGGCTGAGACGAAAAACATATTCTCAATAAACCCTTTA |
| | GGGAAATAGGCCAGGTTTTCACCGTAACACGCCACATCTTGCGAATATATGTGTAGAAACTGCCGGAAAT |
| | CGTCGTGGTATTCACTCCAGAGCGATGAAAACGTTTCAGTTTGCTCATGGAAAACGGTGTAACAAGGGTG |
| | AACACTATCCCATATCACCAGCTCACCGTCTTTCATTGCCATACGGAACTCCGGATGAGCATTCATCAGG |
| | CGGGCAAGAATGTGAATAAAGGCCGGATAAAACTTGTGCTTATTTTTCTTTACGGTCTTTAAAAAGGCCG |
| | TAATATCCAGCTGAACGGTCTGGTTATAGGTACATTGAGCAACTGACTGAAATGCCTCAAAATGTTCTTT |
| | ACGATGCCATTGGGATATATCAACGGTGGTATATCCAGTGATTTTTTTCTCCATTTTAGCTTCCTTAGCT |
| | CCTGAAAATCTCGATAACTCAAAAAATACGCCCGGTAGTGATCTTATTTCATTATGGTGAAAGTTGGAAC |
| | CTCTTACGTGCCGATCAACGTCTCATTTTCGCCAAAAGTTGGCCCAGGGCTTCCCGGTATCAACAGGGAC |
| | ACCAGGATTTATTTATTCTGCGAAGTGATCTTCCGTCACAGGTATTTATTCGGCGCAAAGTGCGTCGGGT |
| | GATGCTGCCAACTTACTGATTTAGTGTATGATGGTGTTTTTGAGGTGCTCCAGTGGCTTCTGTTTCTATC |
| | AGCTGTCCCTCCTGTTCAGCTACTGACGGGGTGGTGCGTAACGGCAAAAGCACCGCCGGACATCAGCGCT |
| | AGCGGAGTGTATACTGGCTTACTATGTTGGCACTGATGAGGGTGTCAGTGAAGTGCTTCATGTGGCAGGA |
| | GAAAAAAGGCTGCACCGGTGCGTCAGCAGAATATGTGATACAGGATATATTCCGCTTCCTCGCTCACTGA |
| | CTCGCTACGCTCGGTCGTTCGACTGCGGCGAGCGGAAATGGCTTACGAACGGGGCGGAGATTTCCTGGAA |
| | GATGCCAGGAAGATACTTAACAGGGAAGTGAGAGGGCCGCGGCAAAGCCGTTTTTCCATAGGCTCCGCCC |
| | CCCTGACAAGCATCACGAAATCTGACGCTCAAATCAGTGGTGGCGAAACCCGACAGGACTATAAAGATAC |
| | CAGGCGTTTCCCCTGGCGGCTCCCTCGTGCGCTCTCCTGTTCCTGCCTTTCGGTTTACCGGTGTCATTCC |
| | GCTGTTATGGCCGCGTTTGTCTCATTCCACGCCTGACACTCAGTTCCGGGTAGGCAGTTCGCTCCAAGCT |
| | GGACTGTATGCACGAACCCCCCGTTCAGTCCGACCGCTGCGCCTTATCCGGTAACTATCGTCTTGAGTCC |
| | AACCCGGAAAGACATGCAAAAGCACCACTGGCAGCAGCCACTGGTAATTGATTTAGAGGAGTTAGTCTTG |
| | AAGTCATGCGCCGGTTAAGGCTAAACTGAAAGGACAAGTTTTGGTGACTGCGCTCCTCCAAGCCAGTTAC |
| | CTCGGTTCAAAGAGTTGGTAGCTCAGAGAACCTTCGAAAAACCGCCCTGCAAGGCGGTTTTTTCGTTTTC |
| | AGAGCAAGAGATTACGCGCAGACCAAAACGATCTCAAGAAGATCATCTTATTAATCAGATAAAATATTTC |
| | TAGATTTCAGTGCAATTTATCTCTTCAAATGTAGCACCTGAAGTCAGCCCCATACGATATAAGTTGTAAT |
| | TCTCATGTTAGTCATGCCCCGCGCCCACCGGAAGGAGCTGACTGGGTTGAAGGCTCTCAAGGGCATCGGT |
| | CGAGATCCCGGTGCCTAATGAGTGAGCTAACTTACATTAATTGCGTTGCGCGAGCTGTTGACAATTAATC |
| | ATCGGCTCGTATAATGTGTGGAATTGTGAGCGGATAACAATTTCACACAGGAGGTAAAAAATGAGCACAA |
| | AAAAGAAACCATTAACACAAGAGCAGCTTGAGGACGCACGTCGCCTTAAAGCAATTTATGAAAAAAAGAA |
| | AAATGAACTTGGCTTATCCCAGGAATCTGTCGCAGACAAGATGGGGATGGGGCAGTCAGGCGTTGGTGCT |
| | TTATTTAATGGCATCAATGCATTAAATGCTTATAACGCCGCATTGCTTACAAAAATTCTCAAAGTTAGCG |
| | TTGAAGAATTTAGCCCTTCAATCGCCAGAGAAATCTACGAGATGTATGAAGCGGTTAGTATGCAGCCGTC |
| | ACTTAGAAGTCAACTTGATATTAATAACACTATAGACCACATATAAAGGAGGTAAAAAAATGATTGAACA |
| | AGATGGATTGCACGCAGGTTCTCCGGCCGCTTGGGTGGAGAGGCTATTCGGCTATGACTGGGCACAACAG |
| | ACAATCGGCTGCTCTGATGCCGCCGTGTTCCGGCTGTCAGCGCAGGGGCGCCCGGTTCTTTTTGTCAAGA |
| | CCGACCTGTCCGGTGCCCTGAATGAACTGCAGGACGAGGCAGCGCGGCTATCGTGGCTGGCCACGACGGG |
| | CGTTCCTTGCGCAGCTGTGCTCGACGTTGTCACTGAAGCGGGAAGGGACTGGCTGCTATTGGGCGAAGTG |
| | CCGGGGCAGGATCTCCTGTCATCTCACCTTGCTCCTGCCGAGAAAGTATCCATCATGGCTGATGCAATGC |
| | GGCGGCTGCATACGCTTGATCCGGCTACCTGCCCATTCGACCACCAAGCGAAACATCGCATCGAGCGAGC |
| | ACGTACTCGGATGGAAGCCGGTCTTGTCGATCAGGATGATCTGGACGAAGAGCATCAGGGGCTCGCGCCA |
| | GCCGAACTGTTCGCCAGGCTCAAGGCGCGCATGCCCGACGGCGAGGATCTCGTCGTGACCCATGGCGATG |
| | CCTGCTTGCCGAATATCATGGTGGAAAATGGCCGCTTTTCTGGATTCATCGACTGTGGCCGGCTGGGTGT |
| | GGCGGACCGCTATCAGGACATAGCGTTGGCTACCCGTGATATTGCTGAAGAGCTTGGCGGCGAATGGGCT |
| | GACCGCTTCCTCGTGCTTTACGGTATCGCCGCTCCCGATTCGCAGCGCATCGCCTTCTATCGCCTTCTTG |
| | ACGCGTTCTTCTAAAGGAGCCTTTCGATGAGTAAAGGAGAAGAACTTTTCACTGGAGTTGTCCCAATTCT |
| | TGTTGAATTAGATGGTGATGTTAATGGGCACAAATTTTCTGTCAGTGGAGAGGGTGAAGGTGATGCAACA |
| | TACGGAAAACTTACCCTTAAATTTATTTGCACTACTGGAAAACTACCTGTTCCATGGCCAACACTTGTCA |
| | CTACTTTCGCGTATGGTCTTCAATGCTTTGCGAGATACCCAGATCATATGAAACAGCATGACTTTTTCAA |
| | GAGTGCCATGCCCGAAGGTTATGTACAGGAAAGAACTATATTTTTCAAAGATGACGGGAACTACAAGACA |
| | CGTGCTGAAGTCAAGTTTGAAGGTGATACCCTTGTTAATAGAATCGAGTTAAAAGGTATTGATTTTAAAG |
| | AAGATGGAAACATTCTTGGACACAAATTGGAATACAACTATAACTCACACAATGTATACATCATGGCAGA |
| | CAAACAAAAGAATGGAATCAAAGTTAACTTCAAAATTAGACACAACATTGAAGATGGAAGCGTTCAACTA |
| | GCAGACCATTATCAACAAAATACTCCAATTGGCGATGGCCCTGTCCTTTTACCAGACAACCATTACCTGT |
| | CCACACAATCTGCCCTTTCGAAAGATCCCAACGAAAAGAGAGATCACATGGTCCTTCTTGAGTTTGTAAC |
| | AGCTGCTGGGATTACACATGGCATGGATGAACTATACAAATAACAACTTGATATTAATAACACTATAACT |
| | TCT |
| 31 | >clmR** sequence that replaces kanR* in SEQ ID NO: 19 for double |
| | reversion experiments. The two point mutations are bold |
| | ATGGAGAAAAAAATCACTGGATATACCACCGTTGATATATCCCAATGGCATCGTAAAGAACATTTTGAGG |
| | CATTTCAGTCAGTTGCTCAATGTACCTATAACCAGACCGTTTAGCTGGATATTACGGCCTTTTTAAAGAC |
| | CGTAAAGAAAAATAAGCACAAGTTTTATCCGGCCTTTATTCACATTCTTGCCCGCCTGATGAATGCTCAT |
| | CCGGAGTTCCGTATGGCAATGAAAGACGGTGAGCTGGTGATATGGGATAGTGTTCACCCTTGTTACACCG |
| | TTTTCCATGAGCAAACTGAAACGTTTTCATCGCTCTGGAGTGAATACCACGACGATTTCCGGCAGTTTCT |
| | ACACATATATTCGCAAGATGTGGCGTGTTACGGTGAAAACCTGGCCTATTTCCCTAAAGGGTTTATTGA |
| | GAATATGTTTTTCGTCTCAGCCAATCCCTGGGTGAGTTTCACCAGTTTTGATTTAAACGTGGCCAATATG |
| | GACAACTTCTTCGCCCCCGTTTTCACTATGGGCAAATATTATACGCAAGGCGACAAGGTGCTGATGCCGC |
| | TGGCGATTCAGGTTCATCATGCCGTCTGTGATGGCTTCCATGTCGGCAGAATGCTTAATGAATTACAACA |
| | GTAATGCGATGAGTGGCAGGGCGGGGCGTAA |
| 32 | > tetA sequence that replaces kanR* in SEQ ID NO: 19 for evolution of |
| | tigecycline resistance |
| | ATGAAATCTAACAATGCGCTCATCGTCATCCTCGGCACCGTCACCCTGGATGCTGTAGGCATAGGCTTGG |
| | TTATGCCGGTACTGCCGGGCCTCTTGCGGGATATCGTCCATTCCGACAGCATCGCCAGTCACTATGGCGT |
| | GCTGCTAGCGCTATATGCGTTGATGCAATTTCTATGCGCACCCGTTCTCGGAGCACTGTCCGACCGCTTT |
| | GGCCGCCGCCCAGTCCTGCTCGCTTCGCTACTTGGAGCCACTATCGACTACGCGATCATGGCGACCACAC |
| | CCGTCCTGTGGATCCTCTACGCCGGACGCATCGTGGCCGGCATCACCGGCGCCACAGGTGCGGTTGCTGG |
| | CGCCTATATCGCCGACATCACCGATGGGGAAGATCGGGCTCGCCACTTCGGGCTCATGAGCGCTTGTTTC |
| | GGCGTGGGTATGGTGGCAGGCCCCGTGGCCGGGGGACTGTTGGGCGCCATCTCCTTGCATGCACCATTCC |
| | TTGCGGCGGCGGTGCTCAACGGCCTCAACCTACTACTGGGCTGCTTCCTAATGCAGGAGTCGCATAAGGG |
| | AGAGCGTCGACCGATGCCCTTGAGAGCCTTCAACCCAGTCAGCTCCTTCCGGTGGGCGCGGGGCATGACT |
| | ATCGTCGCCGCACTTATGACTGTCTTCTTTATCATGCAACTCGTAGGACAGGTGCCGGCAGCGCTCTGGG |
| | TCATTTTCGGCGAGGACCGCTTTCGCTGGAGCGCGACGATGATCGGCCTGTCGCTTGCGGTATTCGGAAT |
| | CTTGCACGCCCTCGCTCAAGCCTTCGTCACTGGTCCCGCCACCAAACGTTTCGGCGAGAAGCAGGCCATT |
| | ATCGCCGGCATGGCGGCCGACGCGCTGGGCTACGTCTTGCTGGCGTTCGCGACGCGAGGCTGGATGGCCT |
| | TCCCCATTATGATTCTTCTCGCTTCCGGCGGCATCGGGATGCCCGCGTTGCAGGCCATGCTGTCCAGGCA |
| | GGTAGATGACGACCATCAGGGACAGCTTCAAGGATCGCTCGCGGCTCTTACCAGCCTAACTTCGATCACT |
| | GGACCGCTGATCGTCACGGCGATTTATGCCGCCTCGGCGAGCACATGGAACGGGTTGGCATGGATTGTAG |
| | GCGCCGCCCTATACCTTGTCTGCCTCCCCGCGTTGCGTCGCGGTGCATGGAGCCGGGCCACCTCGACCTG |
| | A |

1. A recombinant polypeptide comprising a T5 DNA polymerase amino acid sequence operably linked to a DNA helicase amino acid sequence.

2. The recombinant polypeptide of claim 1, wherein the T5 DNA polymerase is an error-prone polymerase.

3. The recombinant polypeptide according to claim 2, wherein the T5 DNA polymerase comprises one or more mutations selected from the group consisting of D164A, E166A, 1308V, and A593R.

4. The recombinant polypeptide according to claim 1, wherein the T5 DNA polymerase amino acid sequence has at least about 80% sequence identity to SEQ ID NO:1, 2, 3, or 4.

5. The recombinant polypeptide according to claim 1, wherein the DNA helicase is Rep helicase or a fragment thereof.

6. The recombinant polypeptide according to claim 1, wherein the DNA helicase amino acid sequence has at least about 80% sequence identity to SEQ ID NO:5 or 20.

7. The recombinant polypeptide according to claim 1, wherein the T5 DNA polymerase amino acid sequence is operably linked to the DNA helicase amino acid sequence via a peptide linker.

8. (canceled)

9. The recombinant polypeptide according to claim 1, comprising an amino acid sequence having at least about 80% sequence identity to SEQ ID NO:10, 11, 22, or 23.

10. (canceled)

11. (canceled)

12. A nucleic acid encoding the recombinant polypeptide of claim 1.

13. An expression cassette comprising the nucleic acid of claim 12, wherein the expression cassette optionally further comprises a 5′-untranslated region (5′ UTR) having at least about 80% sequence identity to SEQ ID NO:16.

14. (canceled)

15. (canceled)

16. A helper vector comprising the nucleic acid sequence of claim 12.

17. A DNA replisome complex, comprising:
1) a target double stranded DNA (dsDNA) comprising a DNA nickase initiation sequence;
2) a corresponding DNA nickase that is capable of nicking the target dsDNA at the initiation sequence; and
3) a recombinant polypeptide according to claim 1,
wherein the recombinant polypeptide is capable of replicating the nicked dsDNA.

18. The DNA replisome complex of claim 17, wherein the DNA nickase is CisA or has an amino acid sequence having at least about 80% sequence identity to SEQ ID NO:24; and/or wherein the DNA nickase initiation sequence has at least about 80% sequence identity to SEO ID NO:13.

19. (canceled)

20. (canceled)

21. (canceled)

22. The DNA replisome complex according to claim 17, wherein the target dsDNA further comprises a target DNA sequence operably linked downstream of the initiation sequence, wherein the target DNA sequence comprises one or more genes.

23. A nucleic acid sequence comprising a target double stranded DNA (dsDNA) as described in claim 17.

24. An expression cassette comprising the nucleic acid sequence of claim 23.

25. A target vector comprising the nucleic acid sequence of claim 23.

26. A host cell comprising the DNA replisome complex according to claim 17.

27. A targeted DNA replication or mutagenesis system comprising:
1) a target dsDNA comprising a DNA nickase initiation sequence;
2) a DNA nickase, or a host cell comprising the DNA nickase; and
3) a recombinant polypeptide of claim 1;
wherein the DNA nickase is capable of nicking the dsDNA or the target vector at the initiation sequence and the recombinant polypeptide is capable of replicating or mutagenizing the nicked dsDNA.

28. A kit comprising:
1) a target vector comprising a DNA nickase initiation sequence;
2) a host cell comprising a DNA nickase (e.g., CisA);
3) a helper vector according to claim 16;
4) packaging materials; and
instructions for inserting a target DNA sequence into the target vector downstream of the DNA nickase initiation sequence; and contacting the host cell with the helper vector and the target vector comprising the target DNA sequence under conditions suitable for the vectors to enter the host cell and mutagenize the target vector.

29. A method comprising contacting a cell with the nucleic acid of claim 12.

30. (canceled)

31. (canceled)

32. The method of claim 29, further comprising contacting the cell with a target dsDNA, wherein the target dsDNA comprises a corresponding DNA nickase initiation sequence operably linked upstream of a target DNA sequence, and optionally, a DNA nickase termination sequence operably linked downstream of the target DNA sequence.

33. A method of mutagenizing a target DNA sequence comprising introducing the target DNA sequence into a target dsDNA downstream of a DNA nickase initiation sequence, and assembling a DNA replisome complex as described in claim 17.

34. (canceled)

35. A method of mutagenizing a target DNA sequence in a cell comprising contacting the target DNA sequence with a recombinant polypeptide as described in claim 1, wherein the target DNA sequence is operably linked downstream of a DNA nickase initiation sequence; and wherein the cell expresses a corresponding DNA nickase; under conditions suitable for the DNA nickase to nick the initiation sequence and for the recombinant polypeptide to mutagenize the target DNA sequence.

36. (canceled)

37. (canceled)

38. (canceled)

39. A method of mutagenizing a target DNA sequence comprising contacting a host cell that expresses a DNA nickase with: 1) a target vector comprising a corresponding DNA nickase initiation sequence operably linked to the target DNA sequence; and 2) a helper vector as described in claim 16; under conditions suitable for the vectors to enter the host cell; for the DNA nickase to nick the initiation sequence; and for the recombinant polypeptide to mutagenize the target DNA sequence.

40. A T5 DNA polymerase comprising a I308V mutation, wherein the substitution and position are in reference to SEQ ID NO:1.

41. (canceled)

42. The T5 DNA polymerase according to claim 40, where in the T5 DNA polymerase comprises an amino acid sequence having at least about 80% sequence identity to SEQ ID NO:3, or 4.


SEQUENCE LISTING (58 sequences)

SEQ ID NO: 1 - protein, 855 aa, Escherichia virus T5
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFDSETSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 2 - protein, 855 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 3 - protein, 855 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDVMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 4 - protein, 855 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFDSETSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDVMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 5 - protein, 672 aa, Unknown
  Description of Unknown Enterobacteriaceae sequence
RLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIWEQERKVVSAEERMQKGQSHLANLKAMMAAKRGK

SEQ ID NO: 6 - protein, 26 aa, Artificial Sequence
  Description of Artificial Sequence Synthetic peptide
VGSKTKEGVVHGVATVAEKTKEQVTN

SEQ ID NO: 7 - protein, 20 aa, Artificial Sequence
  Description of Artificial Sequence Synthetic peptide
GGGGSGGGGSGGGGSGGGGS

SEQ ID NO: 8 - protein, 35 aa, Artificial Sequence
  Description of Artificial Sequence Synthetic polypeptide
GGGASVTGGSAGAGSTVSGAGSIAGSGGGGSGGGG

SEQ ID NO: 9 - protein, 81 aa, Artificial Sequence
  Description of Artificial Sequence Synthetic polypeptide
GGGGSGGGGSGGGGSGGGGSVGSKTKEGVVHGVATVAEKTKEQVTNGGGASVTGGSAGAGSTVSGAGSIAGSGGGGSGGGG

SEQ ID NO: 10 - protein, 1575 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGAGGGGSGGGGSGGGGSGGGGSVGSKTKEGVVHGVATVAEKTKEQVTNGGGASVTGGSAGAGSTVSGAGSIAGSGGGGSGGGGRLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIW

SEQ ID NO: 11 - protein, 1575 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDVMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGAGGGGSGGGGSGGGGSGGGGSVGSKTKEGVVHGVATVAEKTKEQVTNGGGASVTGGSAGAGSTVSGAGSIAGSGGGGSGGGGRLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIW

SEQ ID NO: 12 - DNA, 4728 bp, Artificial Sequence
  Description of Artificial Sequence Synthetic polynucleotide
atgaaaatcgcagtagttgataaagctctaaacaacactcgttatgataaacatttccagctatacggcgaggaagttgatgtattccatatgtgtaacgagaagttgtccggtcgtttgctcaaaaagcatattactatcggaactccggaaaacccatttgacccgaatgattatgattttgttatactggtaggtgccgaacctttcctgtactttgcaggtaagaaaggtattggtgattataccggtaaacgtgtagagtataatggatatgctaactggattgcgagtatcagcccagcccagttacactttaaacctgaaatgaaaccagtttttgatgcaacagtagagaatatccacgatattatcaatggtcgtgagaagattgcaaaagctggtgattaccgtcctattactgaccctgatgaggctgaagaatatatcaagatggtgtataatatggttatcggacccgtcgcattcgcctccgcaacctcagcactatactgtcgagatggttatctgcttggtgtttctatttctcaccaagagtatcagggtgtatatatcgattctgattgtctcacagaggttgcagtatattatctccagaaaattctggatagtgaaaaccacactattgtttttcacaacttgaagtttgatatgcacttttataagtaccatctgggacttacttttgataaagcacataaagaacgcaggctccatgataccatgttgcagcactatgttttagatgaacgtcgtggtactcatggcttgaaatctctagcaatgaagtataccgatatgggtgactatgacttcgaactagataagttcaaagatgattactgtaaagcacataaaatcaagaaagaagatttcacctatgatttaattccgtttgatgttatgtggccatatgctgcgaaagatacggatgccactatacgtttgcacaacttctttttaccaaaaattgagaagaatgaaaaactttgcagtctgtattacgatgttttgatgcctggttgcgtattcttgcaacgtgttgaggatcgtggagtacctatctctattgatcgtttgaaagaagctcagtatcagttgactcataatttgaataaagcccgtgagaaactgtacacttatccagaagttaaacagctagaacaagatcagaatgaagcatttaacccgaactctgttaagcagctacgtgttcttctgtttgattacgttggcttaactccaacaggtaaactgacggatactggagcagattctacggatgcagaagctctaaatgaactggctacgcagcatccaattgctaaaactctgctagagattcgtaagctgactaagctgatctctacttatgttgagaagattctactgagtattgatgcagatggttgcattcgtactggtttccatgaacatatgactacttctggtcgtctgagttcttctggtaaactgaacctgcaacagttaccccgtgatgaatctattatcaagggttgtgtagtagctcctcctgggtatcgtgtaatcgcgtgggacttaacaactgcggaagtttattatgctgctgttctatctggtgatagaaatatgcaacaggtatttatcaacatgagaaatgaacccgataaatacccagacttccactccaacatcgcacacatggtgtttaagctgcaatgcgaaccccgtgatgttaaaaagctgttcccagctctgcgtcaggctgctaaacgcatcaccttcggtattctgtatggttctggcccagctaaagtagcgcattctgttaacgaagcattactagaacaagcagccaagacgggcgaaccgtttgttgaatgtactgttgcagatgctaaagagtacattgagacttacttcggtcagttcccacagcttaagcgttggattgataagtgccacgatcagatcaagaatcatggatttatctatagtcactttggtcgtaaacgtcgtctgcataatatccattccgaagaccgtggtgttcagggtgaagaaattcgttctggatttaatgcaatcattcagtctgcttcttctgatagtctccttttaggtgctgtagatgcagataatgagatcatttctcttggtttagaacaagagatgaagattgttatgttggttcatgactccgtagttgctattgttcgtgaggatttgatcgaccaatacaatgaaatcctgattcgtaatattcagaaagaccgtggtatcagtattcctggctgtccgattggtattgattcagattctgaagctggaggttctcgtgactattcttgtggtaagatgaagaaacagcacccatcaatcgcttgtattgatgatgatgaatatactcgttatgtcaagggtgtattacttgatgcagaattcgagtataagaaactagctgcaatggataaagagcatccagatcatagcaaatacaaggatgataagtttattgctgtatgtaaagatttggataacgtgaaaaggattctcggtgctggaggcggtgggtctggtggtggaggctccggtggcggaggatcagggggaggtggttcggtgggcagtaagacgaaagaaggggtggtgcatggtgtcgcaactgtcgccgaaaaaacaaaggaacaagttactaacggaggcggcgcgtcagttactggtgggagcgctggtgctggatctacagtatcgggcgcgggtagtattgctggctctggtgggggaggttcgggcggcggtggccgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggtggtacgtcgtttttctctcgtcctgaaatcaaggacttgctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcattgacccgcttcactcactggttggcagaaatccagcgtctggcggagcgggagccgattgccgcggtgcgtgatctgatccatggcatggattatgaatcctggctgtacgaaacatcgcccagcccgaaagccgccgaaatgcgcatgaagaacgtcaaccaactgtttagctggatgacggagatgctggaaggcagtgaactggatgagccgatgacgctcacccaggtggtgacgcgctttactttgcgcgacatgatggagcgtggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttggtaa

SEQ ID NO: 13 - DNA, 30 bp, Artificial Sequence
  Description of Artificial Sequence Synthetic oligonucleotide
caacttgatattaataacactatagaccac

SEQ ID NO: 14 - DNA, 30 bp, Artificial Sequence
  Description of Artificial Sequence Synthetic oligonucleotide
caacttgatattaataacactataacttct

SEQ ID NO: 15 - DNA, 18 bp, Artificial Sequence
  Description of Artificial Sequence Synthetic oligonucleotide
taatacgactcactatag

SEQ ID NO: 16 - DNA, 48 bp, Artificial Sequence
  Description of Artificial Sequence Synthetic oligonucleotide
gctctatccagaagttatagtgttattaatattaggttgaggttataa

SEQ ID NO: 17 - DNA, 48 bp, Artificial Sequence
  Description of Artificial Sequence Synthetic oligonucleotide
ctagcataaccccttggggcctctaaacgggtcttgaggggttttttg

SEQ ID NO: 18 - DNA, 7794 bp, Artificial Sequence
  Description of Artificial Sequence Synthetic polynucleotide
taatacgactcactataggctctatccagaagttatagtgttattaatattaggttgaggttataaatgaaaatcgcagtagttgataaagctctaaacaacactcgttatgataaacatttccagctatacggcgaggaagttgatgtattccatatgtgtaacgagaagttgtccggtcgtttgctcaaaaagcatattactatcggaactccggaaaacccatttgacccgaatgattatgattttgttatactggtaggtgccgaacctttcctgtactttgcaggtaagaaaggtattggtgattataccggtaaacgtgtagagtataatggatatgctaactggattgcgagtatcagcccagcccagttacactttaaacctgaaatgaaaccagtttttgatgcaacagtagagaatatccacgatattatcaatggtcgtgagaagattgcaaaagctggtgattaccgtcctattactgaccctgatgaggctgaagaatatatcaagatggtgtataatatggttatcggacccgtcgcattcgcctccgcaacctcagcactatactgtcgagatggttatctgcttggtgtttctatttctcaccaagagtatcagggtgtatatatcgattctgattgtctcacagaggttgcagtatattatctccagaaaattctggatagtgaaaaccacactattgtttttcacaacttgaagtttgatatgcacttttataagtaccatctgggacttacttttgataaagcacataaagaacgcaggctccatgataccatgttgcagcactatgttttagatgaacgtcgtggtactcatggcttgaaatctctagcaatgaagtataccgatatgggtgactatgacttcgaactagataagttcaaagatgattactgtaaagcacataaaatcaagaaagaagatttcacctatgatttaattccgtttgatgttatgtggccatatgctgcgaaagatacggatgccactatacgtttgcacaacttctttttaccaaaaattgagaagaatgaaaaactttgcagtctgtattacgatgttttgatgcctggttgcgtattcttgcaacgtgttgaggatcgtggagtacctatctctattgatcgtttgaaagaagctcagtatcagttgactcataatttgaataaagcccgtgagaaactgtacacttatccagaagttaaacagctagaacaagatcagaatgaagcatttaacccgaactctgttaagcagctacgtgttcttctgtttgattacgttggcttaactccaacaggtaaactgacggatactggagcagattctacggatgcagaagctctaaatgaactggctacgcagcatccaattgctaaaactctgctagagattcgtaagctgactaagctgatctctacttatgttgagaagattctactgagtattgatgcagatggttgcattcgtactggtttccatgaacatatgactacttctggtcgtctgagttcttctggtaaactgaacctgcaacagttaccccgtgatgaatctattatcaagggttgtgtagtagctcctcctgggtatcgtgtaatcgcgtgggacttaacaactgcggaagtttattatgctgctgttctatctggtgatagaaatatgcaacaggtatttatcaacatgagaaatgaacccgataaatacccagacttccactccaacatcgcacacatggtgtttaagctgcaatgcgaaccccgtgatgttaaaaagctgttcccagctctgcgtcaggctgctaaacgcatcaccttcggtattctgtatggttctggcccagctaaagtagcgcattctgttaacgaagcattactagaacaagcagccaagacgggcgaaccgtttgttgaatgtactgttgcagatgctaaagagtacattgagacttacttcggtcagttcccacagcttaagcgttggattgataagtgccacgatcagatcaagaatcatggatttatctatagtcactttggtcgtaaacgtcgtctgcataatatccattccgaagaccgtggtgttcagggtgaagaaattcgttctggatttaatgcaatcattcagtctgcttcttctgatagtctccttttaggtgctgtagatgcagataatgagatcatttctcttggtttagaacaagagatgaagattgttatgttggttcatgactccgtagttgctattgttcgtgaggatttgatcgaccaatacaatgaaatcctgattcgtaatattcagaaagaccgtggtatcagtattcctggctgtccgattggtattgattcagattctgaagctggaggttctcgtgactattcttgtggtaagatgaagaaacagcacccatcaatcgcttgtattgatgatgatgaatatactcgttatgtcaagggtgtattacttgatgcagaattcgagtataagaaactagctgcaatggataaagagcatccagatcatagcaaatacaaggatgataagtttattgctgtatgtaaagatttggataacgtgaaaaggattctcggtgctggaggcggtgggtctggtggtggaggctccggtggcggaggatcagggggaggtggttcggtgggcagtaagacgaaagaaggggtggtgcatggtgtcgcaactgtcgccgaaaaaacaaaggaacaagttactaacggaggcggcgcgtcagttactggtgggagcgctggtgctggatctacagtatcgggcgcgggtagtattgctggctctggtgggggaggttcgggcggcggtggccgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggtggtacgtcgtttttctctcgtcctgaaatcaaggacttgctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcattgacccgcttcactcactggttggcagaaatccagcgtctggcggagcgggagccgattgccgcggtgcgtgatctgatccatggcatggattatgaatcctggctgtacgaaacatcgcccagcccgaaagccgccgaaatgcgcatgaagaacgtcaaccaactgtttagctggatgacggagatgctggaaggcagtgaactggatgagccgatgacgctcacccaggtggtgacgcgctttactttgcgcgacatgatggagcgtggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttggtaactagcataaccccttggggcctctaaacgggtcttgaggggttttttgtccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgattta

SEQ ID NO: 19 - DNA, 4343 bp, Artificial Sequence
  Description of Artificial Sequence Synthetic polynucleotide
ttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgcgagctgttgacaattaatcatcggctcgtataatgtgtggaattgtgagcggataacaatttcacacaggaggtaaaaaatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtcaacttgatattaataacactatagaccacatataaaggaggtaaaaaaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgcgttcttctaaaggagcctttcgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagatcacatggtccttcttgagtttgtaacagctgctgggattacacatggcatggatgaactatacaaataacaacttgatattaataacactataacttct

SEQ ID NO: 20 - protein, 639 aa, Artificial Sequence
  Description of Artificial Sequence Synthetic polypeptide
RLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIW

SEQ ID NO: 21 - DNA, 24 bp, Artificial Sequence
  Description of Artificial Sequence Synthetic oligonucleotide
caacttgatattaataacactata

SEQ ID NO: 22 - protein, 1608 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGAGGGGSGGGGSGGGGSGGGGSVGSKTKEGVVHGVATVAEKTKEQVTNGGGASVTGGSAGAGSTVSGAGSIAGSGGGGSGGGGRLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIWEQERKVVSAEERMQKGQSHLANLKAMMAAKRGK

SEQ ID NO: 23 - protein, 1608 aa, Artificial Sequence
  Description of Artificial Sequence Synthetic polypeptide
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDVMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKRITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGAGGGGSGGGGSGGGGSGGGGSVGSKTKEGVVHGVATVAEKTKEQVTNGGGASVTGGSAGAGSTVSGAGSIAGSGGGGSGGGGRLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGTSFFSRPEIKDLLAYLRVLTNPDDDSAFLRIVNTPKREIGPATLKKLGEWAMTRNKSMFTASFDMGLSQTLSGRGYEALTRFTHWLAEIQRLAEREPIAAVRDLIHGMDYESWLYETSPSPKAAEMRMKNVNQLFSWMTEMLEGSELDEPMTLTQVVTRFTLRDMMERGESEEELDQVQLMTLHASKGLEFPYVYMVGMEEGFLPHQSSIDEDNIDEERRLAYVGITRAQKELTFTLCKERRQYGELVRPEPSRFLLELPQDDLIWEQERKVVSAEERMQKGQSHLANLKAMMAAKRGK

SEQ ID NO: 24 - protein, 513 aa, Unknown
  Description of Unknown Enterobacteria phage phiX174 isolate Sanger sequence
MVRSYYPSECHADYFDFERIEALKPAIEACGISTLSQSPMLGFHKQMDNRIKLLEEILSFRMQGVEFDNGDMYVDGHKAASDVRDEFVSVTEKLMDELAQCYNVLPQLDINNTIDHRPEGDEKWFLENEKTVTQFCRKLAAERPLKDIRDEYNYPKKKGIKDECSRLLEASTMKSRRGFAIQRLMNAMRQAHADGWFIVFDTLTLADDRLEAFYDNPNALRDYFRDIGRMVLAAEGRKANDSHADCYQYFCVPEYGTANGRLHFHAVHFMRTLPTGSVDPNFGRRVRNRRQLNSLQNTWPYGYSMPIAVRYTQDAFSRSGWLWPVDAKGEPLKATSYMAVGFYVAKYVNKKSDMDLAAKGLGAKEWNNSLKTKLSLLPKKLFRIRMSRNFGMKMLTMTNLSTECLIQLTKLGYDATPFNQILKQNAKREMRLRLGKVTVADVLAAQPVTTNLLKFMRASIKMIGVSNLQSFIASMTQKLTLSDISDESKNYLDKAGITTACLRIKSKWTAGGK

SEQ ID NO: 25 - DNA, 1542 bp, Artificial Sequence
  Description of Artificial Sequence Synthetic polynucleotide
atggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccagctggacattaacaataccattgatcatcgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgcttgaagcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggtcacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaacctgcagagttttatcgcttccatgacgcagaagttaacactttcggatatttctgatgagtcgaaaaattatcttgataaagcaggaattactactgcttgtttacgaattaaatcgaagtggactgctggcggaaaatga

SEQ ID NO: 26 - DNA, 2565 bp, Artificial Sequence
  Description of Artificial Sequence Synthetic polynucleotide
atgaaaatcgcagtagttgataaagctctaaacaacactcgttatgataaacatttccagctatacggcgaggaagttgatgtattccatatgtgtaacgagaagttgtccggtcgtttgctcaaaaagcatattactatcggaactccggaaaacccatttgacccgaatgattatgattttgttatactggtaggtgccgaacctttcctgtactttgcaggtaagaaaggtattggtgattataccggtaaacgtgtagagtataatggatatgctaactggattgcgagtatcagcccagcccagttacactttaaacctgaaatgaaaccagtttttgatgcaacagtagagaatatccacgatattatcaatggtcgtgagaagattgcaaaagctggtgattaccgtcctattactgaccctgatgaggctgaagaatatatcaagatggtgtataatatggttatcggacccgtcgcattcgcctccgcaacctcagcactatactgtcgagatggttatctgcttggtgtttctatttctcaccaagagtatcagggtgtatatatcgattctgattgtctcacagaggttgcagtatattatctccagaaaattctggatagtgaaaaccacactattgtttttcacaacttgaagtttgatatgcacttttataagtaccatctgggacttacttttgataaagcacataaagaacgcaggctccatgataccatgttgcagcactatgttttagatgaacgtcgtggtactcatggcttgaaatctctagcaatgaagtataccgatatgggtgactatgacttcgaactagataagttcaaagatgattactgtaaagcacataaaatcaagaaagaagatttcacctatgatttaattccgtttgatgttatgtggccatatgctgcgaaagatacggatgccactatacgtttgcacaacttctttttaccaaaaattgagaagaatgaaaaactttgcagtctgtattacgatgttttgatgcctggttgcgtattcttgcaacgtgttgaggatcgtggagtacctatctctattgatcgtttgaaagaagctcagtatcagttgactcataatttgaataaagcccgtgagaaactgtacacttatccagaagttaaacagctagaacaagatcagaatgaagcatttaacccgaactctgttaagcagctacgtgttcttctgtttgattacgttggcttaactccaacaggtaaactgacggatactggagcagattctacggatgcagaagctctaaatgaactggctacgcagcatccaattgctaaaactctgctagagattcgtaagctgactaagctgatctctacttatgttgagaagattctactgagtattgatgcagatggttgcattcgtactggtttccatgaacatatgactacttctggtcgtctgagttcttctggtaaactgaacctgcaacagttaccccgtgatgaatctattatcaagggttgtgtagtagctcctcctgggtatcgtgtaatcgcgtgggacttaacaactgcggaagtttattatgctgctgttctatctggtgatagaaatatgcaacaggtatttatcaacatgagaaatgaacccgataaatacccagacttccactccaacatcgcacacatggtgtttaagctgcaatgcgaaccccgtgatgttaaaaagctgttcccagctctgcgtcaggctgctaaacgcatcaccttcggtattctgtatggttctggcccagctaaagtagcgcattctgttaacgaagcattactagaacaagcagccaagacgggcgaaccgtttgttgaatgtactgttgcagatgctaaagagtacattgagacttacttcggtcagttcccacagcttaagcgttggattgataagtgccacgatcagatcaagaatcatggatttatctatagtcactttggtcgtaaacgtcgtctgcataatatccattccgaagaccgtggtgttcagggtgaagaaattcgttctggatttaatgcaatcattcagtctgcttcttctgatagtctccttttaggtgctgtagatgcagataatgagatcatttctcttggtttagaacaagagatgaagattgttatgttggttcatgactccgtagttgctattgttcgtgaggatttgatcgaccaatacaatgaaatcctgattcgtaatattcagaaagaccgtggtatcagtattcctggctgtccgattggtattgattcagattctgaagctggaggttctcgtgactattcttgtggtaagatgaagaaacagcacccatcaatcgcttgtattgatgatgatgaatatactcgttatgtcaagggtgtattacttgatgcagaattcgagtataagaaactagctgcaatggataaagagcatccagatcatagcaaatacaaggatgataagtttattgctgtatgtaaagatttggataacgtgaaaaggattctcggtgct

SEQ ID NO: 27 - DNA, 1920 bp, Artificial Sequence
  Description of Artificial Sequence Synthetic polynucleotide
cgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggtggtacgtcgtttttctctcgtcctgaaatcaaggacttgctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcattgacccgcttcactcactggttggcagaaatccagcgtctggcggagcgggagccgattgccgcggtgcgtgatctgatccatggcatggattatgaatcctggctgtacgaaacatcgcccagcccgaaagccgccgaaatgcgcatgaagaacgtcaaccaactgtttagctggatgacggagatgctggaaggcagtgaactggatgagccgatgacgctcacccaggtggtgacgcgctttactttgcgcgacatgatggagcgtggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttggtaa

SEQ ID NO: 28 - DNA, 2019 bp, Escherichia coli
cgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggtggtacgtcgtttttctctcgtcctgaaatcaaggacttgctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcattgacccgcttcactcactggttggcagaaatccagcgtctggcggagcgggagccgattgccgcggtgcgtgatctgatccatggcatggattatgaatcctggctgtacgaaacatcgcccagcccgaaagccgccgaaatgcgcatgaagaacgtcaaccaactgtttagctggatgacggagatgctggaaggcagtgaactggatgagccgatgacgctcacccaggtggtgacgcgctttactttgcgcgacatgatggagcgtggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttgggaacaggagcgcaaagtggtcagcgccgaagaacggatgcagaaagggcaaagccatctggcgaatctgaaagcgatgatggcggcaaaacgagggaaataa

SEQ ID NO: 29 - DNA, 2565 bp, Escherichia virus T5
atgaaaatcgcagtagttgataaagctctaaacaacactcgttatgataaacatttccagctatacggcgaggaagttgatgtattccatatgtgtaacgagaagttgtccggtcgtttgctcaaaaagcatattactatcggaactccggaaaacccatttgacccgaatgattatgattttgttatactggtaggtgccgaacctttcctgtactttgcaggtaagaaaggtattggtgattataccggtaaacgtgtagagtataatggatatgctaactggattgcgagtatcagcccagcccagttacactttaaacctgaaatgaaaccagtttttgatgcaacagtagagaatatccacgatattatcaatggtcgtgagaagattgcaaaagctggtgattaccgtcctattactgaccctgatgaggctgaagaatatatcaagatggtgtataatatggttatcggacccgtcgcattcgactccgaaacctcagcactatactgtcgagatggttatctgcttggtgtttctatttctcaccaagagtatcagggtgtatatatcgattctgattgtctcacagaggttgcagtatattatctccagaaaattctggatagtgaaaaccacactattgtttttcacaacttgaagtttgatatgcacttttataagtaccatctgggacttacttttgataaagcacataaagaacgcaggctccatgataccatgttgcagcactatgttttagatgaacgtcgtggtactcatggcttgaaatctctagcaatgaagtataccgatatgggtgactatgacttcgaactagataagttcaaagatgattactgtaaagcacataaaatcaagaaagaagatttcacctatgatttaattccgtttgatattatgtggccatatgctgcgaaagatacggatgccactatacgtttgcacaacttctttttaccaaaaattgagaagaatgaaaaactttgcagtctgtattacgatgttttgatgcctggttgcgtattcttgcaacgtgttgaggatcgtggagtacctatctctattgatcgtttgaaagaagctcagtatcagttgactcataatttgaataaagcccgtgagaaactgtacacttatccagaagttaaacagctagaacaagatcagaatgaagcatttaacccgaactctgttaagcagctacgtgttcttctgtttgattacgttggcttaactccaacaggtaaactgacggatactggagcagattctacggatgcagaagctctaaatgaactggctacgcagcatccaattgctaaaactctgctagagattcgtaagctgactaagctgatctctacttatgttgagaagattctactgagtattgatgcagatggttgcattcgtactggtttccatgaacatatgactacttctggtcgtctgagttcttctggtaaactgaacctgcaacagttaccccgtgatgaatctattatcaagggttgtgtagtagctcctcctgggtatcgtgtaatcgcgtgggacttaacaactgcggaagtttattatgctgctgttctatctggtgatagaaatatgcaacaggtatttatcaacatgagaaatgaacccgataaatacccagacttccactccaacatcgcacacatggtgtttaagctgcaatgcgaaccccgtgatgttaaaaagctgttcccagctctgcgtcaggctgctaaagcaatcaccttcggtattctgtatggttctggcccagctaaagtagcgcattctgttaacgaagcattactagaacaagcagccaagacgggcgaaccgtttgttgaatgtactgttgcagatgctaaagagtacattgagacttacttcggtcagttcccacagcttaagcgttggattgataagtgccacgatcagatcaagaatcatggatttatctatagtcactttggtcgtaaacgtcgtctgcataatatccattccgaagaccgtggtgttcagggtgaagaaattcgttctggatttaatgcaatcattcagtctgcttcttctgatagtctccttttaggtgctgtagatgcagataatgagatcatttctcttggtttagaacaagagatgaagattgttatgttggttcatgactccgtagttgctattgttcgtgaggatttgatcgaccaatacaatgaaatcctgattcgtaatattcagaaagaccgtggtatcagtattcctggctgtccgattggtattgattcagattctgaagctggaggttctcgtgactattcttgtggtaagatgaagaaacagcacccatcaatcgcttgtattgatgatgatgaatatactcgttatgtcaagggtgtattacttgatgcagaattcgagtataagaaactagctgcaatggataaagagcatccagatcatagcaaatacaaggatgataagtttattgctgtatgtaaagatttggataacgtgaaaaggattctcggtgct

SEQ ID NO: 30 - protein, 513 aa, Artificial Sequence
  Description of Artificial Sequence Synthetic polypeptide
MVRSYYPSECHADYFDFERIEALKPAIEACGISTLSQSPMLGFHKQMDNRIKLLEEILSFRMQGVEFDNGDMYVDGHKAASDVRDEFVSVTEKLMDELAQCYNVLPQLDINNTIDHRPEGDEKWFLENEKTVTQFCRKLAAERPLKDIRDEYNYPKKKGIKDECSRLLEASTMKSRRGFAIQRLMNAMRQAHADGWFIVFDTLTLADDRLEAFYDNPNALRDYFRDIGRMVLAAEGRKANDSHADCYQYFCVPEYGTANGRLHFHAVHFMRTLPTGSVDPNFGRRVRNRRQLNSLQNTWPYGHSMPIAVRYTQDAFSRSGWLWPVDAKGEPLKATSYMAVGFYVAKYVNKKSDMDLAAKGLGAKEWNNSLKTKLSLLPKKLFRIRMSRNFGMKMLTMTNLSTECLIQLTKLGYDATPFNQILKQNAKREMRLRLGKVTVADVLAAQPVTTNLLKFMRASIKMIGVSNLQSFIASMTQKLTLSDISDESKNYLDKAGITTACLRIKSKWTAGGK

SEQ ID NO: 31 - DNA, 660 bp, Artificial Sequence
  Description of Artificial Sequence Synthetic polynucleotide
atggagaaaaaaatcactggatataccaccgttgatatatcccaatggcatcgtaaagaacattttgaggcatttcagtcagttgctcaatgtacctataaccagaccgtttagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtaatgcgatgagtggcagggcggggcgtaa

SEQ ID NO: 32 - DNA, 1191 bp, Artificial Sequence
  Description of Artificial Sequence Synthetic polynucleotide
atgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctga

SEQ ID NO: 33 - protein, 38 aa, Unknown
  Description of Unknown T7 coliphage polymerase sequence
QVVIETAQEAMRWVGDHWNFRCLLDTEGKMGPNWAICH

SEQ ID NO: 34 - protein, 109 aa, Unknown
  Description of Unknown T5 coliphage polymerase sequence
QYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 35 - protein, 34 aa, Escherichia coli
AVAKQIHQLMENCTRLDVPLLVEVGSGENWDQAH

SEQ ID NO: 36 - protein, 35 aa, Thermus aquaticus
AVARLAKEVMEGVYPLAVPLEVEVGIGEDWLSAKE

SEQ ID NO: 37 - protein, 6 aa, Artificial Sequence
  Description of Artificial Sequence Synthetic 6xHis tag
HHHHHH

SEQ ID NO: 38 - protein, 12 aa, Thermus aquaticus
MRRAAKTINFGV

SEQ ID NO: 39 - protein, 5 aa, Artificial Sequence
  Description of Artificial Sequence Synthetic peptide
GGGGS

SEQ ID NO: 40 - protein, 15 aa, Artificial Sequence
  Description of Artificial Sequence Synthetic peptide
GGGGSGGGGSGGGGS

SEQ ID NO: 41 - protein, 4 aa, Artificial Sequence
  Description of Artificial Sequence Synthetic peptide
GGGG

SEQ ID NO: 42 - RNA, 39 nt, Artificial Sequence
  Description of Artificial Sequence Synthetic oligonucleotide
ggcucuauccagguuauaaaugaaaaucgcaguaguuga

SEQ ID NO: 43 - RNA, 69 nt, Artificial Sequence
  Description of Artificial Sequence Synthetic oligonucleotide
ggcucuaucccaacuugauauuaauaacacuauagaccacagguuauaaaugaaaaucgcaguaguuga

SEQ ID NO: 44 - DNA, 69 bp, Artificial Sequence
  Description of Artificial Sequence Synthetic oligonucleotide
  Description of Combined DNA/RNA Molecule Synthetic oligonucleotide
ggcucuauccagaagttatagtgttattaatattaggttgagguuauaaaugaaaaucgcaguaguuga

SEQ ID NO: 45 - RNA, 46 nt, Unknown
  Description of Unknown Wild type G141G sequence
gggcucaugagcgcuuguuucggcguggguaugguggcaggccccg

SEQ ID NO: 46 - RNA, 46 nt, Artificial Sequence
  Description of Artificial Sequence Synthetic oligonucleotide
gggcucaugagcgcuuguuucgguguggguaugguggcaggccccg

SEQ ID NO: 47 - RNA, 38 nt, Unknown
  Description of Unknown Wild type G30G sequence
gcauaggcuugguuaugccgguacugccgggccucuug

SEQ ID NO: 48 - RNA, 38 nt, Artificial Sequence
  Description of Artificial Sequence Synthetic oligonucleotide
gcauaggcuugguuaugccgguacugccgggucucuug

SEQ ID NO: 49 - DNA, 660 bp, Artificial Sequence
  Description of Artificial Sequence Synthetic polynucleotide
atggagaaaaaaatcactggatataccaccgttgatatatcccaatggcatcgtaaagaacattttgaggcatttcagtcagttgctcaatgtacctataaccagaccgtttagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtaatgcgatgagtggcagggcggggcctaa

SEQ ID NO: 50 - DNA, 683 bp, Artificial Sequence
  Description of Artificial Sequence Synthetic polynucleotide
  modified_base (666)..(666): a, c, t, g, unknown or other
  modified_base (670)..(671): a, c, t, g, unknown or other
  modified_base (675)..(683): a, c, t, g, unknown or other
atggagaaaaaaatcactggatataccaccgttgatatatcccaatggcatcgtaaagaacattttgaggcatttcagtcagttgctcaatgtacctataaccagaccgtttggctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagttatgcgatgagtggcagggcggggcctaacaactngatnnaatnnnnnnnnn

SEQ ID NO: 51 - DNA, 682 bp, Artificial Sequence
  Description of Artificial Sequence Synthetic polynucleotide
  modified_base (667)..(667): a, c, t, g, unknown or other
  modified_base (670)..(670): a, c, t, g, unknown or other
  modified_base (675)..(682): a, c, t, g, unknown or other
atggagaaaaaaatcactggatataccaccgttgatatatcccaatggcatcgtaaagaacattttgaggcatttcagtcagttgctcaatgtacctataaccagaccgttaagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagaaatgcgatgagtggcagggcggggcctaacaacttnatntaatnnnnnnnn

SEQ ID NO: 52 - DNA, 683 bp, Artificial Sequence
  Description of Artificial Sequence Synthetic polynucleotide
  modified_base (106)..(106): a, c, t, g, unknown or other
  modified_base (108)..(108): a, c, t, g, unknown or other
  modified_base (670)..(670): a, c, t, g, unknown or other
  modified_base (675)..(683): a, c, t, g, unknown or other
atggagaaaaaaatcactggatataccaccgttgatatatcccaatggcatagtaaagaacattttgaggcatttcagtcagttgctcaatgtacctataaccagncngttaagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagaaatgcgatgagtggcagggcggggcctaacaacttgatntaatnnnnnnnnn

SEQ ID NO: 53 - protein, 12 aa, Unknown
  Description of Unknown T7 coliphage polymerase sequence
GPVAFDSETSAL

SEQ ID NO: 54 - protein, 11 aa, Unknown
  Description of Unknown T5 coliphage polymerase sequence
GVIYDYSTAEY

SEQ ID NO: 55 - protein, 12 aa, Escherichia coli
PVFAFDTETDSL

SEQ ID NO: 56 - protein, 12 aa, Thermus aquaticus
LRQAAKAITFGI

SEQ ID NO: 57 - protein, 12 aa, Unknown
  Description of Unknown T5 coliphage polymerase sequence
TRDNAKTFIYGF

SEQ ID NO: 58 - protein, 12 aa, Escherichia coli
QRRSAKAINFGL